(19) United States
(12) Patent Application Publication
(10) Pub. No.: US 2008/0206768 A1
(43) Pub. Date: Aug. 28, 2008

(54) Predicting a response to olanzapine

(76) Inventor: Maria Arranz, London (GB)

Correspondence Address: Edwards Angell Palmer & Dodge LLP, P.O. Box 55874, Boston, MA, 02205, US

(21) Appl. No.: 12/002,875

(22) Filed: Dec. 18, 2007

(30) Foreign Application Priority Data
Dec. 18, 2006 (GB) 0625194.4
Dec. 18, 2006 (GB) 0625194.6
Jul. 9, 2007 (GB) 0713281.4

(51) Int. Cl. C12Q 1/68

(52) U.S. Cl. 435/6

(57) ABSTRACT

The invention relates generally to the relative effect of specific genetic polymorphisms in predicting the clinical outcome of olanzapine therapy in patients suffering from a psychiatric disease such as schizophrenia.

### TECHNICAL FIELD

The invention relates generally to the relative effect of specific genetic polymorphisms in predicting the clinical outcome of olanzapine therapy in patients suffering from a psychiatric disease such as schizophrenia.

### BACKGROUND FIELD

Psychiatric disorders include anxiety disorders, such as obsessive-compulsive disorder, social phobia, or agoraphobia; eating disorders, including anorexia and bulimia; mood disorders, including manic depression (bipolar disorder); cognitive disorders such as dementias; personality disorders and substance abuse-related disorders; and psychotic disorders, such as schizophrenia and delusional disorders. In general, such disorders are amenable to therapy.
However, psychotic patients typically demonstrate varied responses to treatment with pharmaceutical drugs. Consequently, treatment strategies are trial-and-error, which has a negative effect on morbidity, prognosis and compliance. Methods and products that enable customized drug treatment by identifying genetic components that contribute to the inter-individual differences in drug response and development of drug-induced side effects would improve the quality of care for patients with psychiatric diseases significantly.
Typically, response to drug therapy is measured by a scoring system based on scales which assess a variety of symptoms displayed by psychiatric patients. There are many rating scales used for the measuring of the symptoms and severity of disorders in psychiatry. Examples include the Hamilton Depression Rating Scale (Ham-D), Montgomery-Asberg Depression Rating Scale (MADRS), Young Mania Rating Scale, Hamilton Anxiety Rating Scale (Ham-A), Yale-Brown Obsessive-Compulsive Scale (Y-BOCS), Positive and Negative Syndrome Scale (PANSS) Global Assessment of Functioning (GAF) and Clinical Global Impression (CGI) scales.
Of these, PANSS and GAF can be used to assess schizophrenic disorders, and other psychotic conditions.

### Positive and Negative Syndrome Scale (PANSS)

The PANSS originated as a rigorously operationalised method for evaluating positive, negative, and other symptom dimensions in schizophrenia. The PANSS measurement is derived from behavioural information observed during the interview plus a clinical interview and reports by primary care hospital staff or reports by family members.
The ratings provide summary scores on a 7-item positive scale, a 7-item negative scale and a 16-item general psychopathology scale. The added scores provide a PANSS Total Score.
The PANSS ratings should be based on the totality of information pertaining to a specified period, normally identified as the previous week. Each of the 30 items is accompanied by a specific definition as well as detailed anchoring criteria for all seven rating points. These seven points represent increasing levels of psychopathology, as follows: absent; minimal; mild; moderate; moderate severe; severe; extreme. In assigning ratings, a physician first considers whether a symptom is at all present, as judging by the item definition. If the item is present the physician must determine its severity by reference to the particular criteria for the anchoring points. The highest applicable rating point is always assigned, even if the patient meets criteria for lower ratings as well. The rating points minimal to extreme correspond to incremental levels of symptom severity. They are keyed to the prominence of symptoms, their frequency during the observation phase, and above all their disruptive impact on daily living.

### Global Assessment of Functioning (GAF)

The reporting of overall function on Axis V (5) of the Diagnostic and Statistical Manual of Mental Disorders is performed using the Global Assessment of Functioning (GAF) Scale. The GAF scale may be particularly useful in tracking the clinical progress of individuals in global terms, using a single measure. The GAF scale is to be rated with respect only to psychological and occupational functioning.
The present invention is directed to identifying the relative contributions of genetic polymorphism(s) in a gene or a plurality of genes, to the variability displayed by patients in response to treatment with antipsychotic drugs, in particular olanzapine. Olanzapine is an antipsychotic drug that is well known to those in the art. It is also known as 2-methyl-4-(4-methyl-1-piperazinyl)-10H-thieno[2,3-b][1,5]benzodiazepine. The skilled person appreciates that functional equivalents of olanzapine are encompassed by the invention.

The method according to the invention encompasses genes encoding drug-targeted neurotransmitter receptors, transporters and metabolic enzymes. The products and methods encompassed by the genetic polymorphisms identified herein as contributing to the clinical outcome of olanzapine therapy can facilitate the improvement of antipsychotic treatment.

### SUMMARY OF THE INVENTION

Olanzapine is ineffective, or has sub-optimal efficacy, in treating a significant proportion of individuals with psychotic disorders, and thus a means to determine which patients are likely to benefit from olanzapine treatment is desirable. The present inventors have surprisingly discovered that the presence of specific genetic variations (polymorphism(s)) and combinations thereof, is informative regarding the sensitivity of a patient to olanzapine. Thus, the present invention provides a method for determining the likelihood that a patient will display a response to treatment with olanzapine, based on the patient's genotype.

### Prediction of Response to Olanzapine

There is described a method for determining the likelihood of response to olanzapine in a patient which comprises assessing the genotype of the patient. In the method according to the invention, genetic polymorphisms are analysed in a plurality of genes comprising one or more genes selected from the group consisting of ADRA2A, ADRA1A, ANKK1, D3,5-HTT, M1 and 5-HT6, ChAT, RXFP3, RFXP4 and Neuregulin. Particularly useful combinations of genes are set forth below. In further embodiments, algorithms may be used to predict responsiveness based on polymorphisms present in a plurality of genes comprising one or more genes selected from the foregoing group.
In general, it will be recognised by those skilled in the art that the genes and polymorphisms selected, as well as algorithms eventually used to analyse the polymorphisms, can be selected based on the information available on the patient to be tested and the available data concerning association of particular polymorphisms with responsiveness to olanzapine. Examples are given below.

### Response to Olanzapine Treatment-I

In one embodiment, there is described herein a first example of a method of determining the likelihood of a response to olanzapine treatment in a patient by detecting one or more polymorphisms in a plurality of genes comprising one, two or all three of the following genes of the patient: ADRA2A, ADRA1A and ANKK1. In an aspect of this embodiment, the method further comprises detecting one or more polymorphisms in one, two, three or all four of the following genes of the patient: D3, 5-HTT, M1 and 5-HT6. This method is effective in predicting responsiveness to olanzapine.
In any of these aspects, the polymorphisms detected in ADRA1A include the polymorphism −4155-C/G, where there is a genetic variation in the ADRA1A gene at position −4155. In any of these aspects, the polymorphisms detected in ADRA2A include the polymorphism A/T at position −2211, in which a thymidine replaces adenine. Moreover, in any of these aspects, the polymorphisms detected in ANKK1 include the allelic forms of −8882 C/G. In further embodiments, the polymorphisms detected in D3 include the allelic forms which encode Ser9/Gly9 and/or the polymorphisms detected in 5-HTT include the allelic forms of LPR 480 bp/520 bp, and/or the polymorphisms detected in M1 include the allelic forms of −12064 T/, and/or the polymorphisms detected in 5-HT6 include allelic forms of 267 C/T.

In a preferred embodiment, the detected allelic forms of the one or more polymorphisms in ADRA1A consists of −4155-C/G, wherein said one or more polymorphisms in ADRA2A consists of −2211-A/T, and wherein said one or more polymorphisms in ANKK1 consists of −8882-C/G. In another preferred embodiment, the allelic forms of the polymorphisms detected consist of Ser9/Gly9, wherein said one or more polymorphisms in 5-HTT consists of LPR 480 bp/520 bp, wherein said one or more polymorphisms in M1 consists of −12064-T/C, and wherein said one or more polymorphisms in 5-HT6 consists of 267-C/T.

Variation of the foregoing loci from wild-type is informative of likelihood of response to olanzapine treatment in a patient. Generally, at least three polymorphisms in three different genes should be assayed, preferably including at least one polymorphism from the group set forth above. Preferably, at least two polymorphisms from the group set forth above are assayed, and more preferably at least three. However, different polymorphisms in the same genes can be substituted.

### Response to Olanzapine Treatment-II

In another embodiment, there is described herein a second example of a method of determining the likelihood of a response to olanzapine treatment in a patient by detecting one or more polymorphisms in a plurality of genes comprising one, two or all three of the following genes of the patient: ADRA1A, ANKK1 and M1. In an aspect of this embodiment, the method further comprises detecting the one or more polymorphisms in one, two or three of the following genes of the patient: CHAT, 5HTT, and Neuregulin. This following method is more consistent than the method of the first example.

In any of these aspects, the polymorphisms detected in ADRA1A include −4155 C/G, and/or the polymorphisms detected in ANKK1 include −8882 C/G, and/or the polymorphisms detected in M1 include the allelic forms of 12064 C/G. In further embodiments, the polymorphisms detected in ChAT include rs1880676 G/A, and/or the polymorphisms detected in 5HTT include rs187294 2630 T/C, and/or the polymorphisms detected in Neuregulin include SNP8NRG221533 C/T.

In a preferred embodiment, the polymorphisms detected consist in ADRA1A of −4155 C/G, in ANKK1 of −8882 C/G, and in M1 of −12064 C/G. In another preferred embodiment, the polymorphisms detected in ChAT consist of rs1880676 G/A, and/or the polymorphisms detected in 5HTT consist of rs187294 2630 T/C, and/or the polymorphisms detected in Neuregulin consist of SNP8NRG221533 C/T.

### Algorithms

There are provided algorithms for analysing the observed genotypic differences, as assayed by detection of polymorphisms. Detecting the polymorphisms preferably further includes determining the copy number of the wild type allele with respect to each polymorphism.

Improvement of General Response to Olanzapine treatment

General responsiveness to olanzapine treatment can be assessed clinically, for example by applying the PANSS Total score or the GAF scale. The following algorithms correlate with assessment of effectiveness by measuring general response.

Specifically in one aspect, which corresponds to the first example given above, the likelihood of a response to olanzapine treatment (LoR) in said patient can be predicted using the following algorithm: LoR=[1−(−0.173+1.552A1+1.361A2+2.273B1+1.893B2+0.007C1+0.298C2)], wherein A1=α1A −4155-C/C genotype, A2=α1A −4155-C/G genotype, B1=α2A −2211-A/A genotype, B2=α2A −2211-A/T genotype, C1=ANKK1-8882-C/C genotype, and C2=ANKK1 −8882-C/G genotype.

Specifically in another aspect, the likelihood of response to olanzapine treatment (LoR) in said patient can be predicted using the following algorithm: LoR=[1−(−3.443+1.745A1+1.909A2+2.574B1+1.901B2+0.681C1+1.033C2+1.691D1+0.801D2−18.217E1−18.204E2−-18.589E3+0.732F1+1.099F2+22.508G1+23.778G2)] wherein A1=α1A −4155-C/C genotype, A2=α1A −4155-C/G genotype, B1=α2A −2211-A/A genotype, B2=α2A −2211-A/T genotype, C1=ANKK1 −8882-C/C genotype, C2=ANKK1 −8882-C/G genotype, D1=D3 Ser9/Ser9 genotype, D2=D3 Ser9/Gly9 genotype, E1=5-HTT LPR 480 bp/480 bp genotype, E2=5-HTT LPR 480 bp/520 bp genotype, E3=5-HTT LPR 520 bp/520 bp genotype, F1=M1 −12064-T/T genotype and F2=M1 −12064-T/C genotype, G1=5-HT6267-C/C genotype and G2=5-HT6267-C/T genotype. The sequences of the polymorphic alleles in this algorithm are listed in Table 1A below.

TABLE 1A

Alpha 1A adrenergic receptor −4155 C/G GeneID:148
Rs2644627

(SEQ ID NO:3)

CATCTTTATGACCCCAGAGCATACTC[C/G]TCTCCACTCCACCTACCCA
TGTGTA
Alpha 2A adrenergic receptor −2211 A/T GeneID:150
Rs521674

(SEQ ID NO:4)

AAATATTCTACTCCCTCTTCCCCTTA[A/T]TGAAGGATGCTGTGTGTAC
ATCTGA
ANKK1 −8882 G/C GeneID:255239
Rs3897584

(SEQ ID NO:5)

CTCCCTCCTACATGGCCAAGTACTGA[C/G]GATAGAGCAGAGAACAAGA
TTCCTG
D3 Ser9Gly GeneID:1814
Rs6280

(SEQ ID NO:6)

TGCCCCACAGGTGTAGTTCAGGTGGC[C/T]ACTCAGCTGGCTCAGAGAT
GCCAT
M1 -12,064 T/C GeneID:1128
Rs12295208

(SEQ ID NO:16)

CTGGGGGGCCGTTTGCCCTAGAGATG[C/T]GGGTCCTGCACCGCCTCTG
TTTGG
5-HT6 267 C/G GeneID:3362
Rs1805054

(SEQ ID NO:17)

CCGCCGGCCATGCTGAACGCGCTGTA[C/T]GGGCGCTGGGTGCTGGCGC
GCGG
5-HTT LPR 480/520 bp (SLC6A4) GeneID:6532
See Collier et al., 1996
Forward primer:

(SEQ ID NO:18)

GGC GTT GCC GCT CTG AAT GC
Reverse primer:

(SEQ ID NO:19)

GAG GGA CTG AGC TGG ACA ACC AC

In any of the embodied methods described herein, the response is determined to be beneficial, if there is an improvement of 20 points or more in the GAF scales, or at least a 30% or greater decrease (improvement) in PANSS total score values after olanzapine treatment in the patient.

In another aspect, corresponding to the second example given above and which is consistent in predicting effectiveness of olanzapine as assessed by PANSS total score, determining the likelihood of a general response to olanzapine treatment in said patient (LoR) is calculated according to the following algorithm: [1−(−2.59+0.915A1+0.526A2+1.686B1+1.888B2+0.743C1+1.889C2)] as measured by PANSS, wherein A1=adrenergic receptor rs2644627−4155C/C, A2=Alpha-1A adrenergic receptor rs2644627−-4155C/G, B1=ANKK1 rs3897584−8882C/C, B2=ANKK1 rs3897584-8882 C/G, C1=Muscarinic receptor M1 rs12295208−12064T/T, and C2 Muscarinic receptor M1 rs12295208−12064 C/G.

Specifically in another aspect, the likelihood of a response to olanzapine treatment (LoR) in said patient is calculated according to the following algorithm: =[1−(−5.402+1.602A1+1.108A2+1.212B1+1.965B2+1.399C1+2.814C2+1.118D1+0.068D2+1.708E1+1.952E2+1.097F1−0.806F2)], as measured by PANSS, wherein A1=Alpha-1A adrenergic receptor rs2644627−4155 C/C, A2=Alpha-1A adrenergic receptor rs2644627−4155 C/G, B1=ANKK1 rs3897584−8882 C/C, B2=ANKK1 rs3897584−8882 C/G, C1=Muscarinic receptor M1 rs12295208−12064 T/T, C2=Muscarinic receptor M1 rs122952098−12064 C/G, D1=Choline Acetyltransferase ChAT rs1880676 G/G, D2=Choline Acetyltransferase ChAT rs1880676 G/A, E1=5-HTT rs187294 2630 T/T, E2=5-HTT rs187294 2630 T/C, F1=Neuregulin 1 SNP8NRG221533 C/C and F2=Neuregulin 1 SNP8NRG221533 C/T,

When the response to olanzapine is measured by GAF, and said response is a therapeutically effective response comprises an improvement of 20 points or more in GAF scales, algorithms are provided which predict a patient's responsiveness. For example, in one aspect, determining the likelihood of a general response to olanzapine treatment in a patient (LoR) is calculated according to the following algorithm: (LoR)=[1−(−1.897+1.068A1+0.799A2+1.023B1+1.258B2+0.601C1+1.285C2)], wherein A1=adrenergic receptor rs2644627−4155C/C, A2=Alpha-1A adrenergic receptor rs2644627−4155C/G, B1=ANKK1 rs3897584−8882C/C, B2=ANKK1 rs3897584−8882 C/G, C1=Muscarinic receptor M1 rs12295208−12064T/T, and C2=Muscarinic receptor M1 rs12295208−12064 C/G. Specifically in another aspect, the likelihood of a response to olanzapine treatment (LoR) in said patient can be predicted using the following algorithm(LoR)=[1−(5.916+1.646A1+1.387A2+0.925B1+1.318B2+0.648C1+1.480C2+0.973D1+0.418D2+2.671E1+2.888E2+2.471F1+0.145F2)], wherein A1=Alpha-1A adrenergic receptor rs2644627−4155 C/C, A2=Alpha-1A adrenergic receptor rs2644627−4155 C/G, B1=ANKK1 rs3897584−8882 C/C, B2=ANKK1 rs3897584−8882 C/G, C1=Muscarinic receptor M1 rs12295208−12064 T/T, C2=Muscarinic receptor M1 rs122952098−12064 C/G, D1=Choline Acetyltransferase ChAT rs1880676 G/G, D2=Choline Acetyltransferase ChAT rs1880676 G/A, E1=5-HTT rs187294 2630 T/T, E2=5-HTT rs187294 2630 T/C, F1=Neuregulin 1 SNP8NRG221533 C/C and F2=Neuregulin 1 SNP8NRG221533 C/T.

The sequences of the polymorphic alleles in this algorithm are listed in Table 1B below.

TABLE 1B

Alpha 1A adrenergic receptor −4155 C/G GeneID:148
Rs2644627

(SEQ ID NO:3)

CATCTTTATGACCCCAGAGCATACTC[C/G]TCTCCACTCCACCTACCCA
TGTGTA
ANKK1 −8882 G/C GeneID:255239
Rs3897584

(SEQ ID NO:5)

CTCCCTCCTACATGGCCAAGTACTGA[C/G]GATAGAGCAGAGAACAAGA
TTCCTG
CHAT rs1880676 G/A GeneID:1103
Rs1880676

(SEQ ID NO:24)

CACCAGAGATGTGGCCGGAATGCAGA[A/G]ATGAAGCACTGAGCACAGT
AGGTA
5-HTT 2630 C/T (SLC6A4) GeneID:6532
Rs1872924

(SEQ ID NO:25)

CCAAATGTAGCCACACATCATAGTCA[C/T]CTAgattcctgggtctacc
ccagac
M1 −12,064 T/C GeneID:1128
Rs12295208

(SEQ ID NO:16)

CTGGGGGGCCGTTTGCCCTAGAGATG[C/T]GGGTCCTGCACCGCCTCTG
TTTGG
Neuregulin 1 221533 T/C GeneID:3084
SNP8NRG221533

(SEQ ID NO:27)

ACTAAAAAAGAGATATATGATATTTGG[C/T]AAAATAAAGATACATGGC
TTCCAG

### Improvement in Positive Symptoms by Olanzapine Treatment

In another embodiment, there is described herein a method of determining the likelihood of improvement in positive symptoms by olanzapine treatment in a patient. Positive symptoms can be assessed clinically using, for instance the PANSS positive scale. A reduction of 30% or more in positive PANSS scores is indicative of an improvement in positive symptoms.

Predictions of responsiveness as measured by the PANSS positive scale can be made by detecting the allelic forms of one or more polymorphisms in a plurality of genes comprising one, two, three, of the following genes of the patient: ANKK1, M1 and ADRA1A. In an aspect of this embodiment, the one or more polymorphisms in ANKK1 comprises −8882 C/G, wherein said one or more polymorphisms in M1 comprises −12064 C/G, and wherein said one or more polymorphisms in ADRA1A comprises −4155-C/G.

In one aspect, detecting the allelic forms of the polymorphisms further includes determining the copy number of the wild type allele with respect to each polymorphism. Specifically in one aspect, the likelihood of improvement in positive symptoms on olanzapine treatment in said patient (LoR) is calculated according to the following algorithm: LoR=[1−(−2.08+1.748A1+1.851A2+0.429B1+1.338B2−0.792C1−0.217C2)] wherein A1=ANKK1 rs3897584−8882 C/C, A2=ANKK1 rs3897584−8882 C/G, B1=Muscarinic receptor M1 rs12295208−12064 T/T, B2=Muscarinic receptor M1 rs12295208−12064 T/C, C1=Alpha-1A adrenergic receptor rs2644627−4155 C/C, and C2=Alpha-1A adrenergic receptor rs2644627−4155 C/G. The sequences of the polymorphic alleles in this algorithm are listed in Table 1C below.

TABLE 1C

ANKK1 −8882 G/C GeneID:255239
Rs3897584

(SEQ ID NO:5)

CTCCCTCCTACATGGCCAAGTACTGA[C/G]GATAGAGCAGAGAACAAGA
TTCCTG
Alpha 1A adrenergic receptor −4155 C/G GeneID:148
Rs2644627

(SEQ ID NO:3)

CATCTTTATGACCCCAGAGCATACTC[C/G]TCTCCACTCCACCTACCCA
TGTGTA
M1 −12,064 T/C GeneID:1128
Rs12295208

(SEQ ID NO:16)

CTGGGGGGCCGTTTGCCCTAGAGATG[C/T]GGGTCCTGCACCGCCTCTG
TTTGG

### Improvement in Negative Symptoms by Olanzapine Treatment

In another embodiment, there is described herein a method of determining the likelihood of improvement in negative symptoms by olanzapine treatment in a patient. Negative symptoms can be assessed clinically using, for instance the PANSS negative scale. A reduction of 30% or more in negative PANSS scores is indicatrive of an improvement in negative symptoms.

Predictions of responsiveness as measured by the PANSS negative scale can be made by detecting one or more polymorphisms in a plurality of genes comprising one, two, three or four of the following genes of said patient: RXFP3, RXFP4 and ChAT. In an aspect of this embodiment, the one or more polymorphisms in RXFP3 comprises −903 A/C, the one or more polymorphisms in RXFP4 comprises −3768A/T, and the one or more polymorphisms ChAT comprises rs8178984 C/C.

In one aspect, detecting the allelic forms of the polymorphisms further includes determining the copy number of the wild type allele with respect to each polymorphism. Specifically in one aspect, the likelihood of improvement in negative symptoms by olanzapine treatment in said patient (LoR) is calculated according to the following algorithm: LoR=[1−(−0.221−0.24A1+0.764A2+0.308B1−0.482B2+1.812C1)], wherein A1=RXFP3 rs7702361−903 A/A, A2=RXFP3 rs7702361−903 C/C, B1=RXFP4 rs11264422−3678 A/A, B2=RXFP4 rs11264422−3678 A/T and C1=Choline Acetyltransferase ChAT rs8178984 C/C. The sequences of the polymorphic alleles in this algorithm are listed in Table 1D below.

TABLE 1D

RXFP3 rs7702361 −903-A/C GeneID:51289
Rs7702361

(SEQ ID NO:31)

AGCAGGATAGCTCATATTTTTAGAAC[A/C]ATTCCTCACCAAATGGAAT
AATTCC
RXFP4 rs11264422 −3678-A/T GeneID:339403
Rs11264422

(SEQ ID NO:32)

TTTCTAAACTCAATTCAATAGCACTT[A/T]GTTCTACTGTTAATTGAAA
AACTCT
CHAT rs8178984 C/G GeneID:1103
rs8178984

(SEQ ID NO:33)

GAGCAGAGACTTCCTCAGACCCAACC[C/G]TCTCCAGGATTCAGCAGCA
GCACC

Improvement in “General Psychopathology Symptom Response”

In another embodiment, there is described herein a method of determining the likelihood of improvement in general psychopathology symptoms by olanzapine treatment in a patient. General psychopathology symptoms can be assessed clinically using, for instance the PANSS general psychopathology subscale. A reduction of 30% or more is indicative of an improvement in general psychopathology symptoms.

Predictions of responsiveness as measured by the PANSS general psychopathology subscale can be made detecting one or more polymorphisms in a plurality of genes comprising one, two or three of the following genes of the patient: ChAT, M1 and ANKK1. In an aspect of this embodiment, the one or more polymorphisms in ChAT comprises rs1880676 G/A and the rs8178984 C/G polymorphisms, the one or more polymorphisms in M1 comprises −12064 T/C, and the one or more polymorphisms in ANKK1 comprises −8882 G/C. In another aspect, the one or more polymorphisms in ChAT consist of rs1880676 G/A and the rs8178984 C/G polymorphisms, the one or more polymorphisms in M1 consists of −12064 T/C, and the one or more polymorphisms in ANKK consists of −8882 G/C.

In one aspect, detecting the allelic forms of the polymorphisms further includes determining the copy number of the wild type allele with respect to each polymorphism. Specifically in one aspect, the likelihood of improvement in general psychopathology scores in response to olanzapine treatment in said patient (LoR) is calculated according to the following algorithm: LoR=[1−(−3.461+1.181A1+1.69B]+0.658B2+0.902C1+2.189C2+0.657D1+1.246D2)], wherein A1=Choline Acetyltransferase ChAT rs8178984 C/C, B1=Choline Acetyltransferase ChAT rs1880676 G/G, B2=Choline Acetyltransferase ChAT rs1880676 G/A, C1=Muscarinic receptor M1 rs12295208−12064 T/T, C2=Muscarinic receptor M1 rs12295208−12064 T/C, D1=ANKK1 rs3897584−8882 C/C and D2=ANKK1 rs3897584−8882 C/G. The sequences of the polymorphic alleles in this algorithm are listed in Table 1E below.

TABLE 1E

ANKK1 −8882 G/C GeneID:255239
Rs3897584

(SEQ ID NO:5)

CTCCCTCCTACATGGCCAAGTACTGA[C/G]GATAGAGCAGAGAACAAGA
TTCCTG
CHAT rs1880676 G/A GeneID:1103
Rs1880676

(SEQ ID NO:24)

CACCAGAGATGTGGCCGGAATGCAGA[A/G]ATGAAGCACTGAGCACAGT
AGGTA
M1 −12,064 T/C GeneID:1128
Rs12295208

(SEQ ID NO:16)

CTGGGGGGCCGTTTGCCCTAGAGATG[C/T]GGGTCCTGCACCGCCTCTG
TTTGG
CHAT rs8178984 C/G GeneID:1103
rs8178984

(SEQ ID NO:33)

GAGCAGAGACTTCCTCAGACCCAACC[C/G]TCTCCAGGATTCAGCAGCA
GCACC

In another aspect, there are provided nucleotide sequences encoding any of the above polymorphisms as described herein.

Also described herein is a kit for determining a genotype of an individual, which comprises one or more oligonucleotides that enable detection of a combination of polymorphisms described herein. In one embodiment the genotype of the polymorphisms listed in Tables 1A-1E can be detected using the oligonucleotides listed in Table 2.

TABLE 2

Algorithm SNP Primer Sequences
All sequences 5′-3′

| | |
|---|---|
| ADRA1A -4155 C/G | |
| Forward: | |
| GCC TGT GAC CAT CAA TCC TT | (SEQ ID NO:1) |
| Reverse: | |
| GTC GCA TAG GAG GGT CAG AA | (SEQ ID NO:2) |
| ADRA2A -2211 A/T | |
| F: | |
| TAG CAG GGT GCA AGC TAG TG | (SEQ ID NO:7) |
| R: | |
| GTC ACA TCA GGA AGG GCT CA | (SEQ ID NO:8) |
| ANKK1 -8882 G/C | |
| F: | |
| AAC CTT CAC CCC GGG AGA TT | (SEQ ID NO:9) |
| R: | |
| CTG CAC ATG CAC CCA CTG AA | (SEQ ID NO:10) |
| CHAT rs1880676 | |
| Allele 1: | |
| AGA TGT GGC CGG AAT GCA GAG | (SEQ ID NO:11) |
| Allele 2: | |
| GAG ATG TGG CCG GAA TGC AGA A | (SEQ ID NO:12) |
| Common: | |
| CATACCTACTGTGCTCAGTGCTTCAT | (SEQ ID NO:13) |
| CHAT rs8178984 | |
| F: | |
| AGCGAGCAGAGACTTCCTCA | (SEQ ID NO:14) |
| R: | |
| TAGAAGGGGGCACAGAACAC | (SEQ ID NO:15) |
| DRD3 Ser9Gly | |
| F: | |
| GCT CTA TCT CCA ACT CTC ACA | (SEQ ID NO:22) |
| R: | |
| AAG TCT ACT CAC CTC CAG GTA | (SEQ ID NO:23) |
| 5-HT6 267 C/T | |
| F: | |
| TGC TGA TCG CGC TCA TCT GCA CTC A | (SEQ ID NO:36) |
| R: | |
| CTG CAG CGT CTC CGA GGC CTG ACT G | (SEQ ID NO:37) |
| 5-HTT (SLC6A4) 2630 C/T | |
| F: | |
| TTT CAG AGG AGG CCA AGA GA | (SEQ ID NO:38) |
| R: | |
| TTC AGG AGG TCT GGG GTA GA | (SEQ ID NO:39) |
| 5-HTT (SLC6A4) LPR 480/520 bp | |
| F: | |
| GGC GTT GCC GCT CTG AAT GC | (SEQ ID NO:40) |
| R: | |
| GAG GGA CTG AGC TGG ACA ACC AC | (SEQ ID NO:41) |
| M1 -12,064 T/C | |
| F: | |
| GTGAGAAAGCCCCAGGTTAC | (SEQ ID NO:42) |
| R: | |
| CCAGGCTGGTCTGGACTTCTG | (SEQ ID NO:43) |
| Neuregulin 1 221533 | |
| F: | |
| AAG GCA TCA GTT TTC AAT AGC TTT TT | (SEQ ID NO:44) |
| R: | |
| TAA GTA GAA ATG GGA ACT CTC CAT CTC | (SEQ ID NO:45) |
| Probe 1: | |
| FAM - TTT ATT TTg CCA AAT AT - MGB | (SEQ ID NO:46) |
| Probe 2: | |
| VIC - TCT TTA TTT TaC CAA ATA TCA | (SEQ ID NO:47) |
| T - MGB | |
| RXFP3 rs7702361 A/C | |
| F: | |
| ACG TTG GAT GGA AAG TAA GCA GGA TAG | (SEQ ID NO:48) |
| CTC | |
| R: | |
| ACG TTG GAT GTC CTC ACT CCC ACT TTT | (SEQ ID NO:49) |
| CAC | |
| RXFP4 rs11264422 A/T | |
| F: | |
| ACG TTG GAT GCG GAA TCT GAT TTT CTA | (SEQ ID NO:50) |
| AAC | |
| R: | |
| ACG TTG GAT GCT CAG CCT AAC GTA TCT | (SEQ ID NO:51) |
| CAC | |

In an embodiment of the methods described herein, the procedure for detecting the allelic forms of the polymorphisms is preferably, but not limited to, a procedure selected from the group of: DNA sequencing, allele-specific amplification, and allele-specific primer extension. However, any procedure for detecting the allelic forms of the polymorphisms is encompassed by the invention, including single strand conformation polymorphism (SSCP), denaturing gradient gel electrophoresis (DGGE) or temperature gradient gel electrophoresis analysis (TGGE), mismatch cleavage analysis, cleavage-fragment-length polymorphism analysis (CFLP), denaturing high pressure liquid chromatography (dHPLC), chemical cleavage of mismatch (CCM), Enzymatic cleavage of mismatch (ECM), UNG-mediated T Scan, direct sequencing, DNA chip resequencing, and Pyrosequencing™.

In one embodiment, there is a kit for determining a genotype of an individual, which comprises one or more oligonucleotides that enable detection of one or more or all of the following alleles of the following polymorphisms selected from the group consisting of: wherein said kit comprises oligonucleotides for detection of alleles of each polymorphism in the group consisting of: α1A −4155-C/C genotype, α1A −4155-C/G genotype, α2A −221 ]-A/A genotype, α2A −2211-A/T genotype, ANKK1 −8882-C/C genotype, and ANKK1 −8882-C/G genotype. In one aspect, the oligonucleotides of the kit comprise oligonucleotides with the following sequences: SEQ ID NO:1, SEQ ID NO:2, SEQ ID NO:7, SEQ ID NO:8, SEQ ID NO:9, and SEQ ID NO:10.

In another embodiment, there is a kit for determining a genotype of an individual, which comprises one or more oligonucleotides that enable detection of one or more or all of the following alleles of the following polymorphisms selected from the group consisting of: α1A −4155-C/C genotype, α1A −4155-C/G genotype, α2A −2211-A/A genotype, α1A −2211-A/T genotype, ANKK1 −8882-C/C genotype, ANKK1 −8882-C/G genotype, D3 Ser9/Ser9 genotype, D3 Ser9/Gly9 genotype, 5-HTT LPR 480 bp/480 bp genotype, 5-HTT LPR 480/520 bp genotype, 5-HTT LPR 520/520 bp genotype, M1 −12064-T/T genotype and M1 −12064-T/C genotype, 5-HT6267-C/C genotype and 5-HT6 267-C/T genotypes. In one aspect, the oligonucleotides of the kit comprise oligonucleotides with the following sequences: SEQ ID NO: 1, SEQ ID NO:2, SEQ ID NO:7, SEQ ID NO:8, SEQ ID NO:9, SEQ ID NO: 10, SEQ ID NO:22, SEQ ID NO:23, SEQ ID NO:40, SEQ ID NO:41, SEQ ID NO:42, SEQ ID NO:43, SEQ ID NO:36, and SEQ ID NO:37.

In another embodiment, there is a kit for determining a genotype of an individual, which comprises one or more oligonucleotides that enable detection of one or more or all of the following alleles of the following polymorphisms selected from the group consisting of: Alpha-1A adrenergic receptor rs2644627−4155C/C, Alpha-1A adrenergic receptor rs2644627−4155C/G, ANKK1 rs3897584−8882C/C, ANKK1 rs3897584−8882 C/G, Muscarinic receptor M1 rs12295208−12064 T/T, and Muscarinic receptor M1 rs12295208−12064 T/C. In one aspect, the oligonucleotides of the kit comprise oligonucleotides with the following sequences: SEQ ID NO:1, SEQ ID NO:2, SEQ ID NO:9, SEQ ID NO:10, SEQ ID NO:42, and SEQ ID NO:43.

In another embodiment, there is a kit for determining a genotype of an individual, which comprises one or more oligonucleotides that enable detection of one or more or all of the genotypes of the polymorphisms selected from the group consisting of: Alpha-1A adrenergic receptor rs2644627−4155 C/C, Alpha-1A adrenergic receptor rs2644627−4155 C/G, ANKK1 rs3897584−8882 C/C, ANKK1 rs3897584−8882 C/G, Muscarinic receptor M1 rs12295208−12064 T/T, Muscarinic receptor M1 rs122952098−12064 T/C, Choline Acetyltransferase ChAT rs1880676 G/G, Choline Acetyltransferase ChAT rs1880676 G/A, 5-HTT rs187294 2630 T/T, 5-HTT rs187294 2630 T/C, Neuregulin 1 SNP8NRG221533 C/C and Neuregulin 1 SNP8NRG221533 C/T. In one aspect, the oligonucleotides of the kit comprise oligonucleotides with the following sequences: SEQ ID NO:1, SEQ ID NO:2, SEQ ID NO:9, SEQ ID NO: 10, SEQ ID NO:42, and SEQ ID NO:43, SEQ ID NO:11, SEQ ID NO:12, and SEQ ID NO:13, SEQ ID NO:38, SEQ ID NO:39, SEQ ID NO:44, SEQ ID NO:45, SEQ ID NO:46 and SEQ ID NO:47.

In another embodiment, there is a kit for determining a genotype of an individual, which comprises one or more oligonucleotides that enable detection of one or more or all of the following alleles of the following polymorphisms selected from the group consisting of: ANKK1 rs3897584−8882 C/C, ANKK1 rs3897584−8882 C/G, Muscarinic receptor M1 rs12295208−12064 T/T, Muscarinic receptor M1 rs12295208−12064 T/C, C1=Alpha-1A adrenergic receptor rs2644627−4155 C/C, and C2=Alpha-1A adrenergic receptor rs2644627−4155 C/G. In one aspect, the oligonucleotides of the kit comprise oligonucleotides with the following sequences: SEQ ID NO:9, SEQ ID NO:10, SEQ ID NO:1, SEQ ID NO:2, SEQ ID NO:42 and SEQ ID NO:43.

In another embodiment, there is a kit for determining a genotype of an individual, which comprises one or more oligonucleotides that enable detection of one or more or all of the following alleles of the following polymorphisms selected from the group consisting of: RXFP3 rs7702361−903 A/A, RXFP3 rs7702361−903 C/C, RXFP4 rs11264422−3678 A/A, RXFP4 rs11264422−3678 A/T and Choline Acetyltransferase ChAT rs8178984 C/C genotypes. In one aspect, the oligonucleotides of the kit comprise oligonucleotides with the following sequences: SEQ ID NO:48, SEQ ID NO:49, SEQ ID NO:50, SEQ ID NO:51, SEQ ID NO:14 and SEQ ID NO:15.

In another embodiment, there is a kit for determining a genotype of an individual, which comprises one or more oligonucleotides that enable detection of one or more or all of the following alleles of the following polymorphisms selected from the group consisting of: Choline Acetyltransferase ChAT rs8178984 C/C, Choline Acetyltransferase ChAT rs1880676 G/G, Choline Acetyltransferase ChAT rs1880676 G/A, Muscarinic receptor M1 rs12295208−12064 T/T, Muscarinic receptor M1 rs12295208−12064 T/C, ANKK1 rs3897584−8882 C/C and ANKK1 rs3897584−8882 C/G. In one aspect, the oligonucleotides of the kit comprise oligonucleotides with the following sequences: SEQ ID NO:14, SEQ ID NO:15, SEQ ID NO:11, SEQ ID NO:12, SEQ ID NO: 13 and SEQ ID NO:42, SEQ ID NO:43, SEQ ID NO:9 and SEQ ID NO: 10.

### DETAILED DESCRIPTION

### Definitions

As will become apparent, preferred features and characteristics of one aspect of the invention are applicable to any other aspect of the invention. It should be noted that, as used herein, the singular form “a”, “an” and “the” include plural references unless the context clearly dictates otherwise.
Psychosis: Patients suffering from psychosis have impaired reality testing; that is, they are unable to distinguish personal, subjective experience from the reality of the external world. They experience hallucinations and/or delusions that they believe are real, and may behave and communicate in an inappropriate and incoherent fashion. Psychosis may appear as a symptom of a number of mental disorders and it is the defining feature of schizophrenia, schizophreniform disorder, schizoaffective disorder, delusional disorder, and the psychotic disorders (i.e., brief psychotic disorder, shared psychotic disorder, psychotic disorder due to a general medical condition, and substance-induced psychotic disorder). Functional causes of psychosis also include “mood disorders such as bipolar disorder (manic depression) and severe clinical depression
As used herein, the term “schizophrenia” is intended to include the group of disorders characterized by psychotic symptoms, disruptions in thinking and perception. In a clinical evaluation, schizophrenia is commonly marked by “positive symptoms” such as auditory hallucinations (ie hearing voices), disorganized thought processes and delusions as well as “negative symptoms” which include affective flattening, alogia, avolition, and anhedonia.
As used herein “the positive symptoms of schizophrenia” refer to a class of symptoms of schizophrenia characterised by hallucinations (sensory perception in the absence of external stimuli) which may occur in any of the five senses, although tend to be auditory.
They are a common feature of psychosis and are prominent and often distressing. Positive symptoms also include delusional or paranoid false beliefs that are usually incomprehensible in terms of normal mental processes yet held with conviction by the patient. Thought disorder describes an underlying disturbance to conscious thought and is classified largely by its effects on speech and writing.
As used herein, “the negative symptoms of schizophrenia” refer to a class of symptoms of schizophrenia which can be considered to reflect a ‘loss’ in functional, directed thought or activity. Negative symptoms of schizophrenia are well known in the art, and include affective flattening (characterized by, for example, an immobile and/or unresponsive facial expression, poor eye contact and reduced body language), alogia (‘poverty of speech’ or brief, laconic and/or empty replies), avolition (characterized by a reduced or absent ability to initiate and carry out goal-directed activities), anhedonia (loss of interest or pleasure), social withdrawal, apathy and other negative symptoms known to those of skill in the art.
The symptoms and general functioning of patients with schizophrenia are assessed using a variety of published rating scales.
Ratings may be made in terms of overall symptoms or in terms of level of functioning, or in terms of specific symptom groups.
The negative symptoms of schizophrenia may be assessed using any methodology known in the art including, but not limited to, the Brief Psychiatric Rating Scale (BPRS), the Positive and Negative Syndrome Scale (PANSS), and the Scale for the Assessment of Negative Symptoms (SANS). Some of these methods may also be used to assess positive symptoms (e.g., BPRS, PANSS), although methods for specifically assessing positive symptoms are also available (e.g., the Scale for the Assessment of Positive Symptoms (SAPS)).
General symptoms of psychopathology associated with psychotic illness (such as somatic concern, anxiety, guilt feelings, tension) may also be assessed (e.g. by the PANSS scale).
The symptoms or symptom classes/subgroups of psychosis may be assessed separately e.g as individual scale items, or as subscales, (e.g., negative symptom scale of PANSS, positive symptom scale of PANSS, general psychopathology scale of PANSS,) or combined to give a total overall assessment of symptoms (e.g Total PANSS score).
An overall assessment of symptoms and functioning may be obtained by other scales including but not limited to the GAF (Global assessment of Functioning scale) and the CGI (Clinical Global Impression scale).
As used herein, the terms “response to olanzapine treatment” includes pharmacological effectiveness. Pharmacological effectiveness refers to the ability of the treatment to result in a desired clinical effect in the patient.
As used herein “olanzapine treatment” refers to a course of treatment encompassing administration of olanzapine to a patient in therapeutically effective dose(s) over a time period. In one embodiment the time period is three months or more, up to and including, 6 months, a year, three years or longer. However the time period can also be shorter than three months. All the methods of treating described herein include administration of olanzapine or a olanzapine related molecule-by any method known to those skilled in the art including subcutaneous, intramuscular, intradermal, transdermal, intraperitoneal, intravenous, intranasal, intrathecal, intraocular, or oral routes of administration.
In one embodiment, a psychotic patient's response to olanzapine treatment response was assessed prospectively using the PANSS and GAF scales. In an aspect of this embodiment, a “positive response to olanzapine treatment” means a reduction in the symptoms of the psychotic disease, and in one embodiment is evidenced by an improvement of at least 20 points or more in the GAF scales, or at least a 30% decrease in PANSS values after olanzapine treatment. A positive response may also encompass an improvement in specific symptoms of a psychotic disease. This may include an improvement in positive symptoms, and/or negative symptoms and/or a general psychpathology symptoms response. The more positive the response, the more the symptoms are reduced. These patients are classified as “Responders”.
As used herein the term “a negative response to olanzapine treatment” means the treatment provides no reduction of the assessed symptoms of the psychotic disease, or causes an increase in the symptoms of the psychotic disease being treated. The more negative the response, the more the symptoms are increased. These patients are classified as “non-responders”.
A patient may be an overall responder as measured by GAF or PANSS total score, but may still, for example, be a negative symptom non-responder if the improvement in the negative symptoms to olanzapine treatment fails to meet the response criteria.
As used herein, phrase “likelihood of a response” to olanzapine treatment means the probability that a patient will display the response after olanzapine treatment. Probability can be measured in terms of percentage, ranging from 0 to 100%: if the percentage is low, then there is a low likelihood that the patient will have the response of interest, and conversely, where the percentage is high, there is a higher likelihood or probability that the patient will display the response of interest. Expression of psychotic diseases is multifactorial, hence it is unlikely to achieve a probability of 100% based on hereditary factors alone. Accordingly, the phrase “determining the likelihood of a response” to olanzapine provides an approximate probability that a patient with a particular genotype at specific polymorphic loci will display the response to olanzapine being measured.
The term “genotype” in the context of this invention refers to the particular combination of allelic forms of a gene, which can be defined by the particular nucleotide(s) present in a nucleic acid sequence at a particular site(s).
The terms “polymorphism”, “genotype”, “variant form of a gene”, “form of a gene” or “allele” refer to one specific form of a gene in a population, the specific form differing from other forms of the same gene in the sequence of at least one, and frequently more than one, variations from wild type within the sequence of the gene. The sequences at these sites of variation within a gene that differ between alleles of the gene are termed “gene sequence polymorphisms” or “polymorphisms” or “variants” or “allelic variants”. Other terms known in the art to be equivalent include mutation and polymorphism. The polymorphisms may be single or multiple base changes, including without limitation insertions, deletions, or substitutions, or may be a variable number of sequence repeats.
In one aspect, the term “Allele” refers to normal alleles of a locus as well as alleles of the gene carrying variations that affect responsiveness to olanzapine. In preferred aspects of this invention, the polymorphisms are selected from the group consisting of the polymorphisms listed in Tables 1A-1E.
“Isolated” or “substantially pure”. An “isolated” or “substantially pure” nucleic acid (e.g., an RNA, DNA or a mixed polymer) is one which is substantially separated from other cellular components which naturally accompany a native human sequence or protein, e.g., ribosomes, polymerases, many other human genome sequences and proteins. The term embraces a nucleic acid sequence or protein which has been removed from its naturally occurring environment, and includes recombinant or cloned DNA isolates and chemically synthesized analogs or analogs biologically synthesized by heterologous systems.
“Encode” A polynucleotide is said to “encode” a polypeptide if, in its native state or when manipulated by methods well known to those skilled in the art, it can be transcribed and/or translated to produce the mRNA for and/or the polypeptide or a fragment thereof. The anti-sense strand is the complement of such a nucleic acid, and the encoding sequence can be deduced there from.
The term “promoter sequence” or “promoter” defines a single strand of a nucleic acid sequence that is specifically recognized by an RNA polymerase that binds to a recognized sequence and initiates the process of transcription by which an RNA transcript is produced. In principle, any promoter sequence may be employed for which there is a known and available polymerase that is capable of recognizing the initiation sequence. Known and useful promoters are those that are recognized by certain bacteriophage polymerases, such as bacteriophage T3, T7 or SP6.
“Regulatory sequences” refers to those sequences normally within 100 kb of the coding region of a gene, but they may also be more distant from the coding region, which affect the expression of the gene (including transcription of the gene, and translation, splicing, stability or the like of the messenger RNA).
The term “label” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means. The term “support” refers to conventional supports such as beads, particles, dipsticks, fibers, filters, membranes and silane or silicate supports such as glass slides.
A “microarray” is a linear or two-dimensional array of preferably discrete regions, each having a defined area, formed on the surface of a solid support. The density of the discrete regions on a microarray is determined by the total numbers of target polynucleotides to be detected on the surface of a single solid phase support, preferably at least about 50/cm², more preferably at least about 100/cm², even more preferably at least about 500/cm², and still more preferably at least about 1,000/cm². As used herein, a DNA microarray is an array of oligonucleotide primers placed on a chip or other surfaces used to amplify or clone target polynucleotides. Since the position of each particular group of primers in the array is known, the identities of the target polynucleotides can be determined based on their binding to a particular position in the microarray.
As used herein, a “sample” refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), organs, and also samples of in vitro cell culture constituent.
The term “amplify” is used in the broad sense to mean creating an amplification product which may include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases. Any of several techniques that increase the number of copies of a nucleic acid molecule. A preferred example of amplification is the polymerase chain reaction (PCR), in which a sample containing the nucleic acid is contacted with a pair of oligonucleotide primers under conditions that allow for the hybridization of the primers to nucleic acid in the sample. The primers are extended under suitable conditions, dissociated from the template, and then re-annealed, extended, and dissociated to amplify the number of copies of the nucleic acid. The amplification products (called “amplicons”) can be further processed, manipulated, or characterized by (without limitation) electrophoresis, restriction endonuclease digestion, hybridization, nucleic acid sequencing, ligation, or other techniques of molecular biology. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in European Patent Appl. 320 308; gap filling ligase chain reaction amplification, as disclosed in U.S. Pat. No. 5,427,930; and RNA transcription-free amplification, as disclosed in U.S. Pat. No. 6,025,134.
The term “primer”, as used herein, refers to an oligonucleotide which is capable of acting as a point of initiation of polynucleotide synthesis along a complementary strand when placed under conditions in which synthesis of a primer extension product which is complementary to a polynucleotide is catalyzed. Such conditions include the presence of four different nucleotide triphosphates or nucleoside analogs and one or more agents for polymerization such as DNA polymerase and/or reverse transcriptase, in an appropriate buffer (“buffer” includes substituents which are cofactors, or which affect pH, ionic strength, etc.), and at a suitable temperature. A primer must be sufficiently long to prime the synthesis of extension products in the presence of an agent for polymerase. A typical primer contains at least about 5 nucleotides in length of a sequence substantially complementary to the target sequence, but somewhat longer primers are preferred. Usually primers contain about 15-26 nucleotides, but longer primers may also be used.
A primer will always contain a sequence substantially complementary to the target sequence, that is the specific sequence to be amplified, to which it can anneal. A primer may, optionally, also comprise a promoter sequence. Primers are useful to amplify sequences from the region of the polymorphism and are preferably complementary to, and hybridize specifically to sequences that flank one or more polymorphisms in a gene. Polymorphic sequences generated by amplification may be sequenced directly or may be cloned prior to sequence analysis. A method for the direct cloning and sequence analysis of enzymatically amplified genomic segments has been described by Scharf et al., 1986. In the context of this invention, the term “probe” refers to a molecule which can detectably distinguish between target molecules differing in structure, such as allelic variants. Detection can be accomplished in a variety of different ways but preferably is based on detection of specific binding. Examples of such specific binding include antibody binding and nucleic acid probe hybridization. Thus, for example, probes can include enzyme substrates, antibodies and antibody fragments, and preferably nucleic acid hybridization probes.
“Polynucleotide Probes”. Polynucleotide polymorphisms associated with alleles which contribute to the sensitivity of a patient's response to olanzapine treatment can be detected by hybridization with a polynucleotide probe which forms a stable hybrid with that of the target sequence, under stringent to moderately stringent hybridization and wash conditions. If it is expected that the probes will be perfectly complementary to the target sequence, high stringency conditions will be used. Hybridization stringency may be lessened if some mismatching is expected, for example, if variants are expected with the result that the probe will not be completely complementary. Conditions are chosen which rule out nonspecific/adventitious bindings, that is, which minimize noise. For techniques for preparing and labeling probes see, e.g., Sambrook et al., 1989 or Ausubel et al., 1992. Probes comprising synthetic oligonucleotides or other polynucleotides of the present invention may be derived from naturally occurring or recombinant single- or double-stranded polynucleotides, or be chemically synthesized. Probes may also be labeled by nick translation, Klenow fill-in reaction, or other methods known in the art.
The term “gene” as used herein is a polynucleotide which may include coding sequences, intervening sequences and regulatory elements controlling transcription and/or translation. The term “gene” as used herein is intended to encompass all allelic variations of the gene's DNA sequence. Genes of the invention refer to those genes that are likely to be expressed in normal tissue, certain alleles of which contribute to a patient's response to olanzapine. As used herein a gene encompasses a polynucleotide which encodes a polypeptide, fragment, homolog or variant, including, e.g., protein fusions or deletions or insertions. The nucleic acids of the present invention will possess a sequence which is either derived from, or has substantial homology with a natural encoding gene which contributes to a patient's response to olanzapine, or a portion thereof.
Genes of the invention include normal alleles of the gene encoding polymorphisms that contribute to a patient's sensitivity to olanzapine, including silent alleles having no effect on the amino acid sequence of the gene's encoded polypeptide as well as alleles leading to amino acid sequence variants of the encoded polypeptide that do not substantially affect its function or its contribution to responsiveness to olanzapine therapy. These terms also include alleles having one or more mutations which affect the function of the encoded polypeptide's its contribution to responsiveness to olanzapine therapy.
The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.
The present invention provides recombinant nucleic acids comprising all or part of a gene encoding a polymorphism contributing to the sensitivity of a patient's response to olanzapine treatment. The recombinant construct may be capable of replicating autonomously in a host cell. Alternatively, the recombinant construct may become integrated into the chromosomal DNA of the host cell. Such a recombinant polynucleotide comprises a polynucleotide of genomic, cDNA, semi-synthetic, or synthetic origin which, by virtue of its origin or manipulation, 1) is not associated with all or a portion of a polynucleotide with which it is associated in nature; 2) is linked to a polynucleotide other than that to which it is linked in nature; or 3) does not occur in nature. Where nucleic acid according to the invention includes RNA, reference to the sequence shown should be construed as reference to the RNA equivalent, with U substituted for T.
Therefore, recombinant nucleic acids comprising sequences otherwise not naturally occurring are provided by this invention. Although the wild-type sequence may be employed, it will often be altered, e.g., by deletion, substitution or insertion. cDNA or genomic libraries of various types may be screened as natural sources of the nucleic acids of the present invention, or such nucleic acids may be provided by amplification of sequences resident in genomic DNA or other natural sources, e.g., by PCR. The choice of cDNA libraries normally corresponds to a tissue source which is abundant in mRNA for the desired proteins. Phage libraries are normally preferred, but other types of libraries may be used. Clones of a library are spread onto plates, transferred to a substrate for screening, denatured and probed for the presence of desired sequences. The methods of nucleic acid isolation, amplification and analysis are routine for one skilled in the art and examples of protocols can be found, for example, in the Molecular Cloning: A Laboratory Manual (3-Volume Set) Ed. Joseph Sambrook, David W. Russel, and Joe Sambrook, Cold Spring Harbor Laboratory; 3rd edition (Jan. 15, 2001), ISBN: 0879695773. Particularly useful protocol source for methods used in PCR amplification is PCR (Basics: From Background to Bench) by M. J. McPherson, S. G. Moller, R. Beynon, C. Howe, Springer Verlag; 1st edition (Oct. 15, 2000), ISBN: 0387916008.
“Substantial homology or similarity”. A nucleic acid or fragment thereof is “substantially homologous” (“or substantially similar”) to another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases. To determine homology between two different nucleic acids, the percent homology is to be determined using the BLASTN program “BLAST 2 sequences”. This program is available for public use from the National Center for Biotechnology Information (NCBI) over the Internet (http://www.ncbi.nlm.nih.gov/gorf/b12.html) (Altschul et al., 1997).
“Kit” refers to a combination of physical elements, e.g., probes, including without limitation specific primers, labeled nucleic acid probes, antibodies, protein-capture agent(s), reagent(s), instruction sheet(s) and other elements useful to practice the invention, in particular identify the alleles present in a polymorphism. These physical elements can be arranged in any way suitable for carrying out the invention. For example, probes and/or primers can be provided in one or more containers or in an array or microarray device.
Methods of Detecting Alleles of Polymorphisms Associated with Olanzapine
An allele associated with a response to olanzapine treatment can be detected by any of a variety of available techniques, including: 1) performing a hybridization reaction between a nucleic acid sample and a probe that is capable of hybridizing to the allele; 2) sequencing at least a portion of the allele; or 3) determining the electrophoretic mobility of the allele or fragments thereof (e.g., fragments generated by endonuclease digestion). The allele can optionally be subjected to an amplification step prior to performance of the detection step. Preferred amplification methods are selected from the group consisting of: the polymerase chain reaction (PCR), the ligase chain reaction (LCR), strand displacement amplification (SDA), cloning, and variations of the above (e.g. RT-PCR and allele specific amplification). Oligonucleotides necessary for amplification may be selected for example, from within the gene loci containing the polymorphism of interest, either flanking the polymorphism of interest (as required for PCR amplification) or directly overlapping the polymorphism of interest (as in allele specific oligonucleotide (ASO) hybridization). In a particularly preferred embodiment, the sample is hybridized with a set of primers, which hybridize 5′ and 3′ in a sense or antisense sequence to the allele of interest, and is subjected to a PCR amplification.
Allele-specific oligonucleotide (ASO) hybridization as described by Henri WAJCMAN, MD, Ph.D on the URL://rbc.gs-im3.fr/DATA/The %20HW_CD/EnglASO.html Dec. 12, 2007
Two Methods for Diagnosis are Based on this Approach:
1. The dot-blotting method requires the binding of the PCR amplified target DNA sequence to a nylon membrane. The DNA fixed to the membrane is then hybridized to the allele specific oligonucleotide probes that are 5′ end-labelled with either ³²P-labelled deoxynucleoside triphosphates, biotin, horseradish peroxidase or a fluorescent marker.
For mutation screening, a panel of ASO probes is required that needs to be adapted to the mutations found in the ethnic group of the individual, which is tested.
For genotyping homozygous patients, two oligonucleotide probes are required for each mutation: one complimentary to the mutant DNA sequence and the other complimentary to the normal gene sequence at the same position.
The patient's genotype is determined by analysis of the presence or absence of the hybridisation signal of both the mutation specific and normal probe. The technique is used with great success for investigation of populations with just one common mutation and a small number of rare ones. However, this method is not adapted to screening populations carrying a large number of different mutations since each mutation requires a separate hybridization and washing step.
The reverse dot-blotting technique allows several mutations to be tested in a single hybridization reaction. In this method, in contrast to the previous one, unlabelled ASO probes, specific to various mutations and to the normal DNA sequence, are bound to a nylon membrane strips in the form of dots or slots. A labelled amplified genomic DNA is then hybridized to the filter. This procedure may require the use of several filters, the first one corresponds to the more frequent mutations observed in the patient's ethnic and the others to less frequent abnormalities.
An allele of interest may also be detected indirectly, e.g. by analyzing the protein product encoded by the DNA. For example, where the polymorphism in question results in the translation of a variable protein, the protein can be detected by any of a variety of protein detection methods. Such methods include immunodetection and biochemical tests, such as size fractionation, where the protein has a change in apparent molecular weight either through truncation, elongation, altered folding or altered post-translational modifications. Such immunodetectin methods include enzyme linked immunosorbent assays (ELISA), radioimmunoassays (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530, hereby incorporated by reference.
Alleles Associated with Response to Olanzapine
An allele whose presence is identified with individuals responding to treatment with olanzapine, either alone or in combination with other alleles at different polymorphisms, is encompassed herein. Examples of these type of alleles are listed in Table 1A through 1E.

### Kits and Diagnostic Products and Methods

The present invention is useful in a diagnostic product to detect the presence of olanzapine sensitive allele(s). Accordingly, the invention encompasses the use of diagnostic kits based on a variety of methodologies, e.g., sequence, chip, mass-spectroscopy, which are capable of finding allelic sequences indicative of the polymorphic alleles described herein, e.g. Table 1. The invention also provides an article of manufacturing comprising packaging material and a pharmaceutical agent contained within the packaging material, wherein the pharmaceutical agent comprises means for detecting the presence of one or more alleles of a polymorphism associated with olanzapine response, and packaging material comprises a label or package insert which indicates that the detection means can be used to identify a candidate subject suitable or unsuitable for treatment of a psychiatric disease such as schizophrenia with olanzapine.
The present invention therefore also provides predictive and prognostic kits comprising degenerate primers to amplify polymorphic alleles associated with a response to olanzapine in a patient and instructions comprising an amplification protocol and analysis of the results. The kit may alternatively also comprise buffers, enzymes, and containers for performing the amplification and analysis of the amplification products. The kit may also be a component of a screening or prognostic kit comprising other tools such as DNA microarrays. Preferably, the kit also provides one or more control templates, such as nucleic acids isolated from normal tissue sample, and/or a series of samples representing different polymorphisms in the same gene or in different genes associated with a response to olanzapine.
The kit may also include instructions for use of the kit to amplify specific targets on a solid support. Where the kit contains a prepared solid support having a set of primers already fixed on the solid support, e.g. for amplifying a particular set of target polynucleotides, the kit also includes reagents necessary for conducting a PCR on a solid support, for example using an in situ-type or solid phase type PCR procedure where the support is capable of PCR amplification using an in situ-type PCR machine. The PCR reagents, included in the kit, include the usual PCR buffers, a thermostable polymerase (e.g. Taq DNA polymerase), nucleotides (e.g. dNTPs), and other components and labeling molecules (e.g. for direct or indirect labeling). The kits can be assembled to support practice of the PCR amplification method using immobilized primers alone or, alternatively, together with solution phase primers.
In one embodiment, the kit provides two or more primer pairs, each pair capable of amplifying a different region of a gene associated with olanzapine response and/or multiple polymorphisms from a plurality of genes, thereby providing a kit for analysis of expression of several gene polymorphisms in a biological sample in one reaction or several parallel reactions. Primers in the kits may be labeled, for example fluorescently labeled, to facilitate detection of the amplification products and consequent analysis of the nucleic acid variances.
In one embodiment, more than one polymorphism can be detected in one analysis. A combination kit will therefore comprise of primers capable of amplifying different segments of a single gene. The primers may be differentially labeled, for example using different fluorescent labels, so as to differentiate between the variances.
The primers contained within the kit may include those listed in Table 2, and various subcombinations thereof.

### Method of Treating a Patient

In one embodiment, the invention provides a method for selecting or guiding the selection of a treatment for a patient affected by a psychotic disease by determining the genotype of at least one polymorphism in the patient. In a preferred embodiment, the genotype of a plurality of polymorphisms in the patient is determined, whereby a plurality may include variances from one, two, three or more gene loci. For even greater specificity, an analysis of a second subset of polymorphisms associated with a response to olanzapine is undertaken.
In certain embodiments, the presence of at least one allelic variation from wild type in a polymorphism associated with olanzapine treatment is indicative that the treatment will be effective or otherwise beneficial (or more likely to be beneficial) in the patient. Stating that the treatment will be effective means that the probability of beneficial therapeutic effect is greater than in a person not having the above referenced allelic variation. Alternatively the wild type allele may be indicative of an effective olanzapine treatment.
Table 1 is a partial list of DNA sequence polymorphisms in genes relevant to the methods described in the present invention. These polymorphisms were identified as having alleles which influenced a patient's response to olanzapine by the inventors in studies of biological samples from patients with psychotic disorders who were exposed to olanzapine therapy the algorithms do not predict side effects, we do not have that information from the samples where the studies were carried out
This will require continued mutational analyses and identification of additional genes and polymorphisms which contribute to a patient's response to olanzapine. With more detailed phenotypic analyses, phenotypic differences between the varied forms of patient responsiveness to olanzapine, such as improvement in negative symptoms, or improvement in positive symptoms may be discovered. These differences may be useful to further modify therapeutic treatment, and extend the analysis and treatment to other populations. A “population” refers to a defined group of individuals or a group of individuals with a particular disease or condition or individuals that may be treated with a specific drug identified by, but not limited to geographic, ethnic, race, gender, and/or cultural indices. In most cases a population will preferably encompass at least ten thousand, one hundred thousand, one million, ten million, or more individuals, with the larger numbers being more preferable. In preferred embodiments of this invention, the population refers to individuals with a specific disease or condition that may be treated with a specific drug.
The present invention is further detailed in the following Examples, which are offered by way of illustration and are not intended to limit the invention in any manner. Standard techniques well known in the art or the techniques specifically described below are utilized.

### EXAMPLES

### Materials and Methods

The mathematical algorithms were calculated using information of individual genetic association studies performed on clinical samples. After gathering information on which genetic polymorphisms may be associated with treatment variability, a mathematical algorithm was produced by logistic regression that allocates a predictive value or coefficient to each relevant genetic polymorphism according to their contribution to response variability. The prediction score was obtained by multiplying the coefficients by the genotypes present in an individual. The resulting value gives an indication of the likelihood of response.

### Example 1

### Clinical Samples

Olanzapine clinical sample: 147 subjects (129 with schizophrenia or schizo-affective disorder, 4 with bipolar disorder, 4 with major depression and 3 with atypical psychosis) were recruited in Navarra (Northern Spain) and were of Basque and Spanish origin. All subjects were treated with the antipsychotic olanzapine for a minimum of 3 months. Treatment response was assessed prospectively using the PANSS(2) and GAF scales. Improvement of 20 points or more in the GAF scales, or at least a 30% decrease in PANSS values on olanzapine treatment was considered as the threshold for response.
DNA was extracted from whole blood samples using standard methods.

### Example 2

### Genotyping of Predictive Polymorphisms

Polymorphisms of interest were genotyped using PCR amplification using the primers described in Table 2. The skilled person understands that the conditions and protocols used for the detection of the predictive polymorphisms are not relevant to the invention and can be easily modified and adapted to accommodate the systems/technology available in most laboratories.

### Example 3

Calculation of Prediction algorithms
The clinical samples were genotyped for all the polymorphisms of interest listed in table 1. This information was then combined to produce a predictive algorithm for olanzapine as follows: Logistic regression was calculated considering response to olanzapine treatment as the predicted bimodal (response or non-response) variable. For algorithms predicting improvement in positive, negative or general psychpathology psymptoms, bimodal variables based on at least a 30% decrease in overall, positive, negative or general psychopathology symptoms were created respectively. Logistic regression was then calculated using the appropriate response variable. After performing logistic regression analyses, an algorithm was produced in which the genotypes of the predictor polymorphisms were multiplied by a coefficient according to their contribution to response variability. The equation algorithm is described below:

### Example 4

### Equation Algorithm for Genetic Prediction of Response to Olanzapine, as Assessed on the GAF Scale

Logistic regression on olanzapine response, calculated on the olanzapine sample described above, and using as predictor variables genetic polymorphisms in the alpha-2A adrenergic receptor (α_{2A}), the alpha-1A adrenergic receptor (α_{1A}) and the ANKK1 (Ankyrin repeat and kinase domain containing-1) genes produced the following result:

Likelihood of olanzapine response (LoR)=[1−(−0.173+1.552A1+1.361A2+2.273B1+1.893B2+0.007C1+0.298C2)]

### Where:

A1=α_{1A}−4155-C/C genotype
A2=α_{1A}−4155-C/G genotype
B1=α_{2A}−2211-A/A genotype
B2=α_{2A}−2211-A/T genotype
C1=ANKK1 −8882-C/C genotype
C2=ANKK1 −8882-C/G genotype
The result of the logistic regression is a probability (%) of the likelihood of response or non-response. For example, a value of 0.2 will indicate a 20% chance of responding (showing an increase of at least 20 GAF score points) to treatment with olanzapine given for at least 3 months.
This algorithm had the following statistic values:
Level of correct prediction: 72.2%
Positive predictive value=66%
Negative predictive value=74.5%

### Sensitivity=53.3%

### Specificity=83.7%

TABLE 3
Classification table

| PREDICTED AND OBSERVED | | Predicted response | | % |
|---|---|---|---|---|
| RESPONSE TO OLANZAPINE | | Responder | Non-responder | Correct |
| Observed | Responder | 16 | 14 | 53.3 |
| response | Non-responder | 8 | 41 | 83.7 |

| | |
|---|---|
| Overall % of correctly predicted outcomes | 72.2 |

Cut value in table=0.5 (i.e. the probability of a positive response of 50% was used to distinguish likely responders from likely non-responders)

TABLE 4
Variables in the Equation

| | | B | S.E. | Wald | df | Sig. | Exp(B) |
|---|---|---|---|---|---|---|---|
| Step | a1a4155 | | | 6.000 | 2 | .050 | |
| 1^{a} | a1a4155(1) | 1.552 | .826 | 3.534 | 1 | .060 | 4.721 |
| | a1a4155(2) | 1.361 | .598 | 5.184 | 1 | .023 | 3.898 |
| | a2a2211 | | | 3.515 | 2 | .172 | |
| | a2a2211(1) | 2.273 | 1.223 | 3.452 | 1 | .063 | 9.710 |
| | a2a2211(2) | 1.893 | 1.207 | 2.462 | 1 | .117 | 6.641 |
| | ankk1.84 | | | .332 | 2 | .847 | |
| | ankk1.84(1) | .007 | 1.016 | .000 | 1 | .994 | 1.007 |
| | ankk1.84(2) | .298 | 1.014 | .086 | 1 | .769 | 1.347 |
| | Constant | −.173 | .533 | .105 | 1 | .745 | .841 |

^{a}Variable(s) entered on step 1: a1a4155, a2a2211, ankk1.84.

### I

### Example 5

An extended version of the foregoing algorithm incorporating information on genetic variants of 5-HTT, α_{1A}, α_{2A}, ANKK1, D3, M1 and 5-HT6 genes was also calculated, and produced the following result:

LoR=[1−(−3.443+1.745A1+1.909A2+2.574B1+1.901B2+0.681C1+1.033C2+1.691D1+0.801D2−18.217E1−18.204E2−18.589E3+0.732F1+1.099F2+22.508G1+23.778G2)]

### Where:

A1=α_{1A}−4155-C/C genotype
A2=α_{1A}−4155-C/G genotype
B1=α_{2A}−2211-A/A genotype
B2=α_{2A}−2211-A/T genotype
C1=ANKK1 −8882-C/C genotype
C2=ANKK1 −8882-C/G genotype
D1=D3 Ser9/Ser9 genotype
D2=D3 Ser9/Gly9 genotype
E1=5-HTT LPR 480 bp/480 bp genotype
E2=5-HTT LPR 480/520 bp genotype
E3=5-HTT LPR 520/520 bp genotype
F1=M1 −12064-T/T genotype
F2=M1 −12064-T/C genotype
G1=5-HT6 267-C/C genotype
G2=5-HT6 267-C/T genotype
This algorithm had the following statistical values:
Level of prediction: 80%
Positive predictive value=77%
Negative predictive value=81%

### Sensitivity=63%

### Specificity=89%

TABLE 5
Classification table

| PREDICTED AND OBSERVED | | Predicted response | | % |
|---|---|---|---|---|
| RESPONSE TO OLANZAPINE | | Responder | Non-responder | Correct |
| Observed | Responder | 17 | 10 | 63.0 |
| response | Non-responder | 5 | 43 | 89.6 |

| | |
|---|---|
| Overall % of correctly predicted outcomes | 80.0 |

cut value in table=0.50 (i.e. the probability of a positive response of 50% was used to distinguish likely responders from likely non-responders)

TABLE 6
Variables in the Equation

| | | B | S.E. | Wald | df | Sig. | Exp(B) |
|---|---|---|---|---|---|---|---|
| Step | a1a4155 | | | 5.502 | 2 | .064 | |
| 1^{a} | a1a4155 | 1.745 | 1.056 | 2.731 | 1 | .098 | 5.725 |
| | (1) | | | | | | |
| | a1a4155 | 1.909 | .833 | 5.257 | 1 | .022 | 6.747 |
| | (2) | | | | | | |
| | a2a2211 | | | 3.941 | 2 | .139 | |
| | a2a2211 | 2.574 | 1.318 | 3.816 | 1 | .051 | 13.116 |
| | (1) | | | | | | |
| | a2a2211 | 1.901 | 1.256 | 2.292 | 1 | .130 | 6.692 |
| | (2) | | | | | | |
| | ankk1.84 | | | .887 | 2 | .642 | |
| | ankk1.84 | .681 | 1.189 | .329 | 1 | .566 | 1.977 |
| | (1) | | | | | | |
| | ankk1.84 | 1.033 | 1.168 | .781 | 1 | .377 | 2.809 |
| | (2) | | | | | | |
| | d3msci | | | 3.297 | 2 | .192 | |
| | d3msci | 1.691 | 1.052 | 2.583 | 1 | .108 | 5.423 |
| | (1) | | | | | | |
| | d3msci | .801 | 1.089 | .541 | 1 | .462 | 2.229 |
| | (2) | | | | | | |
| | m1tsp45i | | | 1.840 | 2 | .398 | |
| | m1tsp45i | .732 | .835 | .767 | 1 | .381 | 2.079 |
| | (1) | | | | | | |
| | m1tsp45i | 1.099 | .810 | 1.840 | 1 | .175 | 3.000 |
| | (2) | | | | | | |
| | ht6c267t | | | 1.132 | 2 | .568 | |
| | ht6c267t | 22.508 | 40192.878 | .000 | 1 | 1.000 | 6.0E+09 |
| | (1) | | | | | | |
| | ht6c267t | 23.778 | 40192.878 | .000 | 1 | 1.000 | 2.1E+10 |
| | (2) | | | | | | |
| | sertn | | | .230 | 3 | .973 | |
| | sertn(1) | −18.217 | 40193.008 | .000 | 1 | 1.000 | .000 |
| | sertn(2) | −18.204 | 40193.008 | .000 | 1 | 1.000 | .000 |
| | sertn(3) | −18.589 | 40193.008 | .000 | 1 | 1.000 | .000 |
| | Constant | −3.443 | 16747.056 | .000 | 1 | 1.000 | .032 |

^{a}Variable(s) entered on step 1: a1a4155, a2a2211, ankk1.84, d3msci, m1tsp45i, ht6c267t, sertn.

### Example 6

Using a strategy encompassing a combination of information in polymorphisms/genes that had shown association with clinical outcome in response to olanzapine treatment the algorithms for the prediction of olanzapine response (as measured by GAF scales) detailed in the above examples were developed.
Additional genotyping in the samples has been performed and new algorithms have been formulated as described below. The algorithms differ from the core and extended algorithms described in Examples 1-5, in the combination of polymorphisms (although there may be some polymorphisms common to both) and in the weighting given to each polymorphism.

### Materials and Methods

The mathematical algorithms were calculated using information of individual genetic association studies performed on clinical samples. After gathering information on which genetic polymorphisms may be associated with treatment variability, a mathematical algorithm was produced by logistic regression that allocates a predictive value or coefficient to each relevant genetic polymorphism according to their contribution to response variability. The prediction score was obtained by multiplying the coefficients by the genotypes present in an individual. The resulting value gives an indication of the likelihood of response.

### Clinical Samples

147 subjects (129 with schizophrenia or schizo-affective disorder, 4 with bipolar disorder, 4 with major depression and 3 with atypical psychosis) were recruited in Navarra (Northern Spain) and were of Basque and Spanish origin. All subjects were treated with the antipsychotic olanzapine for a minimum of 3 months. Treatment response was assessed prospectively using the PANSS(2) and GAF scales. Improvement of 20 points or more in the GAF scales, or at least a 30% decrease in PANSS values after olanzapine treatment was considered as the threshold for response. At least a 30% decrease in positive, negative or general psychopathology symptoms was considered as threshold for improvement in positive, negative or general psychopathology symptoms, respectively.
A core algorithm (including the most reliable combination of genes) and an extended algorithm (including additional SNPs that may give an improved prediction level) determining the likelihood of general response (as measured by GAF and PANSS, two response measurement scales that measure different outcomes) have been calculated. The combination of genes used for the core algorithms is more reliable than the combination used for the extended algorithms, which are likely to change significantly when larger samples are investigated.
Olanzapine CORE algorithm for the prediction of total PANSS scores response

TABLE 7
Classification Table^{a}

| | | | Predicted | | |
|---|---|---|---|---|---|
| | | | olanzapine response | | |
| | | | (30% reduction in total | | |
| | | | panss) | | Per- |
| | | | re- | non-re- | centage |
| | Observed | | sponder | sponder | Correct |
| Step | olanzapine response | responder | 17 | 21 | 44.7 |
| 1 | (30% reduction in | non- | 9 | 53 | 85.5 |
| | total panss) | responder | | | |
| | Overall Percentage | | | | 70.0 |

^{a}The cut value is .500

TABLE 8
Variables in the Equation

| | | B | S.E. | Wald | df | Sig. | Exp(B) |
|---|---|---|---|---|---|---|---|
| Step | a1a4155 | | | 1.694 | 2 | .429 | |
| 1^{a} | a1a4155(1) | .915 | .739 | 1.532 | 1 | .216 | 2.496 |
| | a1a4155(2) | .526 | .538 | .952 | 1 | .329 | 1.691 |
| | ankk1.84 | | | 5.212 | 2 | .074 | |
| | ankk1.84(1) | 1.686 | .821 | 4.217 | 1 | .040 | 5.400 |
| | ankk1.84(2) | 1.888 | .836 | 5.102 | 1 | .024 | 6.607 |
| | m1tsp45i | | | 8.787 | 2 | .012 | |
| | m1tsp45i(1) | .743 | .605 | 1.511 | 1 | .219 | 2.103 |
| | m1tsp45i(2) | 1.889 | .664 | 8.092 | 1 | .004 | 6.610 |
| | Constant | −2.590 | 1.014 | 6.516 | 1 | .011 | .075 |

^{a}Variable(s) entered on step 1: a1a4155, ankk1.84, m1tsp45i.

Ditto above re names of SNPs

Likelihood of response (LoR)=[1−(−2.59+0.915A1+0.526A2+1.686B1+1.888B2+0.743C+1.889C2)]

### Specificity=85.5%

### Sensitivity=44.7%

Positive predictive value (PPV)=65.4%
Negative predictive value (NPV)=71.6%
whereas:
A1=Alpha-1A adrenergic receptor rs2644627−4155C/C
A2=Alpha-1A adrenergic receptor rs2644627−4155C/G
B1=ANKK1 rs3897584−8882C/C
B2=ANKK1 rs3897584−8882 C/G
C1=Muscarinic receptor M1 rs12295208−12064T/T
C2=Muscarinic receptor M1 rs12295208−12064 T/C

### Olanzapine CORE Algorithm for the Prediction of Overall Response (as Measured by GAF)

TABLE 9
Classification Table^{a}

| | | | Predicted | | |
|---|---|---|---|---|---|
| | | | Olanzapine response | | |
| | | | (20 points GAS) | | Per- |
| | | | re- | non-re- | centage |
| | Observed | | sponder | sponder | Correct |
| Step | Olanzapine response | responder | 14 | 24 | 36.8 |
| 1 | (20 points GAS) | non- | 9 | 55 | 85.9 |
| | | responder | | | |
| | Overall Percentage | | | | 67.6 |

^{a}The cut value is .500

TABLE 10
Variables in the Equation

| | | B | S.E. | Wald | df | Sig. | Exp(B) |
|---|---|---|---|---|---|---|---|
| Step | a1a4155 | | | 3.116 | 2 | .211 | |
| 1^{a} | a1a4155(1) | 1.068 | .704 | 2.307 | 1 | .129 | 2.911 |
| | a1a4155(2) | .799 | .517 | 2.388 | 1 | .122 | 2.224 |
| | ankk1.84 | | | 2.671 | 2 | .263 | |
| | ankk1.84(1) | 1.023 | .759 | 1.819 | 1 | .177 | 2.782 |
| | ankk1.84(2) | 1.258 | .770 | 2.669 | 1 | .102 | 3.518 |
| | m1tsp45i | | | 4.482 | 2 | .106 | |
| | m1tsp45i(1) | .601 | .597 | 1.012 | 1 | .314 | 1.823 |
| | m1tsp45i(2) | 1.285 | .626 | 4.214 | 1 | .040 | 3.615 |
| | Constant | −1.897 | .934 | 4.126 | 1 | .042 | .150 |

^{a}Variable(s) entered on step 1: a1a4155, ankk1.84, m1tsp45i.

Ditto SNP names

Likelihood of response (LoR)=[1−(−1.897+1.068A1+0.799A2+1.023B1+1.258B2+0.601C1+1.285C2)]

### Specificity=85.9%

### Sensitivity=36.8%

Positive predictive value (PPV)=60.9%
Negative predictive value (NPV)=69.6%
whereas:
A1=Alpha-1A adrenergic receptor rs2644627−4155 C/C
A2=Alpha-1A adrenergic receptor rs2644627−4155 C/G
B=ANKK1 rs3897584−8882 C/C
B2=ANKK1 rs3897584−8882 C/G
C1=Muscarinic receptor M1 rs12295208−12064 T/T
C2=Muscarinic receptor M1 rs12295208−12064 T/C

### Olanzapine EXTENDED Algorithm for the Prediction of Total PANSS Scores Response

TABLE 11
Classification Table^{a}

| | | | Predicted | | |
|---|---|---|---|---|---|
| | | | olanzapine response | | |
| | | | (30% reduction in total | | |
| | | | panss) | | Per- |
| | | | re- | non-re- | centage |
| | Observed | | sponder | sponder | Correct |
| Step | olanzapine response | responder | 17 | 15 | 53.1 |
| 1 | (30% reduction in | non- | 8 | 43 | 84.3 |
| | total panss) | responder | | | |
| | Overall Percentage | | | | 72.3 |

^{a}The cut value is .500

TABLE 12
Variables in the Equation

| | | B | S.E. | Wald | df | Sig. | Exp(B) |
|---|---|---|---|---|---|---|---|
| Step | a1a4155 | | | 3.578 | 2 | .167 | |
| 1^{a} | a1a4155(1) | 1.602 | .921 | 3.024 | 1 | .082 | 4.965 |
| | a1a4155(2) | 1.108 | .700 | 2.504 | 1 | .114 | 3.028 |
| | ankk1.84 | | | 4.205 | 2 | .122 | |
| | ankk1.84(1) | 1.212 | .957 | 1.604 | 1 | .205 | 3.359 |
| | ankk1.84(2) | 1.965 | .993 | 3.917 | 1 | .048 | 7.135 |
| | m1tsp45i | | | 9.916 | 2 | .007 | |
| | m1tsp45i(1) | 1.399 | .758 | 3.408 | 1 | .065 | 4.052 |
| | m1tsp45i(2) | 2.814 | .899 | 9.798 | 1 | .002 | 16.685 |
| | chatch6 | | | 2.891 | 2 | .236 | |
| | chatch6(1) | 1.118 | .882 | 1.604 | 1 | .205 | 3.058 |
| | chatch6(2) | .068 | .834 | .007 | 1 | .935 | 1.070 |
| | htt18729 | | | 2.979 | 2 | .225 | |
| | htt18729(1) | 1.708 | 1.139 | 2.248 | 1 | .134 | 5.517 |
| | htt18729(2) | 1.952 | 1.131 | 2.979 | 1 | .084 | 7.042 |
| | nrg1533 | | | 4.566 | 2 | .102 | |
| | nrg1533(1) | 1.097 | 1.007 | 1.188 | 1 | .276 | 2.996 |
| | nrg1533(2) | −.806 | .634 | 1.614 | 1 | .204 | .447 |
| | Constant | −5.402 | 1.928 | 7.850 | 1 | .005 | .005 |

^{a}Variable(s) entered on step 1: a1a4155, ankk1.84, m1tsp45i, chatch6, htt18729, nrg1533.

LoR=[1−(−5.402+1.602A1+1.108A2+1.212B1+1.965B2+1.399C1+2.814C2+1.118D1+0.068D2+1.708E1+1.952E2+1.097F1−0.806F2)]

### Specificity=84.3%

### Sensitivity=53.1%

Positive predictive value (PPV)=68.0%
Negative predictive value (NPV)=74.1%
whereas:
A1=Alpha-1A adrenergic receptor rs2644627−4155 C/C
A2=Alpha-1A adrenergic receptor rs2644627−4155 C/G
B1=ANKK1 rs3897584−8882 C/C
B2=ANKK1 rs3897584−8882 C/G
C1=Muscarinic receptor M1 rs12295208−12064 T/T
C2=Muscarinic receptor M1 rs122952098−12064 T/C
D1=Choline Acetyltransferase ChAT rs1880676 G/G
D2=Choline Acetyltransferase ChAT rs1880676 G/A
E1=5-HTT rs187294 2630 T/T
E2=5-HTT rs187294 2630 T/C

### F1=Neuregulin 1 SNP8NRG221533 C/C

### F2=Neuregulin 1 SNP8NRG221533 C/T

### Olanzapine EXTENDED Algorithm for the Prediction of Overall Response (as Measured by GAF)

TABLE 13
Classification Table^{a}

| | | | Predicted | | |
|---|---|---|---|---|---|
| | | | Olanzapine response | | |
| | | | (20 points GAS) | | Per- |
| | | | re- | non-re- | centage |
| | Observed | | sponder | sponder | Correct |
| Step | Olanzapine response | responder | 16 | 15 | 51.6 |
| 1 | (20 points GAS) | non- | 4 | 50 | 92.6 |
| | | responder | | | |
| | Overall Percentage | | | | 77.6 |

^{a}The cut value is .500

TABLE 14
Variables in the Equation

| | | B | S.E. | Wald | df | Sig. | Exp(B) |
|---|---|---|---|---|---|---|---|
| Step | a1a4155 | | | 5.054 | 2 | .080 | |
| 1^{a} | a1a4155(1) | 1.646 | .891 | 3.416 | 1 | .065 | 5.187 |
| | a1a4155(2) | 1.387 | .672 | 4.260 | 1 | .039 | 4.002 |
| | ankk1.84 | | | 2.000 | 2 | .368 | |
| | ankk1.84(1) | .925 | .952 | .944 | 1 | .331 | 2.522 |
| | ankk1.84(2) | 1.318 | .950 | 1.923 | 1 | .165 | 3.736 |
| | m1tsp45i | | | 3.795 | 2 | .150 | |
| | m1tsp45i(1) | .648 | .729 | .790 | 1 | .374 | 1.911 |
| | m1tsp45i(2) | 1.480 | .784 | 3.563 | 1 | .059 | 4.394 |
| | chatch6 | | | 1.604 | 2 | .449 | |
| | chatch6(1) | .973 | .823 | 1.400 | 1 | .237 | 2.647 |
| | chatch6(2) | .418 | .797 | .275 | 1 | .600 | 1.519 |
| | htt18729 | | | 4.916 | 2 | .086 | |
| | htt18729(1) | 2.671 | 1.325 | 4.065 | 1 | .044 | 14.454 |
| | htt18729(2) | 2.888 | 1.303 | 4.916 | 1 | .027 | 17.965 |
| | nrg1533 | | | 4.611 | 2 | .100 | |
| | nrg1533(1) | 2.471 | 1.183 | 4.362 | 1 | .037 | 11.837 |
| | nrg1533(2) | .145 | .574 | .064 | 1 | .800 | 1.157 |
| | Constant | −5.916 | 2.039 | 8.419 | 1 | .004 | .003 |

^{a}Variable(s) entered on step 1: a1a4155, ankk1.84, m1tsp45i, chatch6, htt18729, nrg1533.

LoR=[1−(5.916+1.646A1+1.387A2+0.925B1+1.318B2+0.648C1+1.480C2+0.973D1+0.418D2+2.671E1+2.888E2+2.471F1+0.145F2)]

### Specificity=92.6%

### Sensitivity=51.6%

Positive predictive value (PPV)=80.0%
Negative predictive value (NPV)=76.9%
whereas:
A1=Alpha-1A adrenergic receptor −4155 C/C
A2=Alpha-1A adrenergic receptor −4155 C/G
B1=ANKK1 rs3897584−8882 C/C
B2=ANKK1 rs3897584−8882 C/G
C1=Muscarinic receptor M1 rs12295208−12064 T/T
C2=Muscarinic receptor M1 rs12295208−12064 T/C
D1=Choline Acetyltransferase ChAT rs1880676 G/G
D2=Choline Acetyltransferase ChAT rs1880676 G/A
E1=5-HTT rs187294 2630 T/T
E2=5-HTT rs187294 2630 T/C

### F1=Neuregulin 1 SNP8NRG221533 C/C

### F2 Neuregulin 1 SNP8NRG221533 C/T

### Olanzapine Algorithm for the Prediction of Improvement in Positive Symptoms (as Measured by PANSS Positive Scale)

TABLE 15
Classification Table^{a}

| | | | Predicted | | |
|---|---|---|---|---|---|
| | | | ppanssRNR | | Per- |
| | | | Re- | Non-Re- | centage |
| | Observed | | sponder | sponder | Correct |
| Step | ppanssRNR | Responder | 35 | 15 | 70.0 |
| 1 | | Non- | 21 | 29 | 58.0 |
| | | Responder | | | |
| | Overall Percentage | | | | 64.0 |

^{a}The cut value is .500

TABLE 16
Variables in the Equation

| | | B | S.E. | Wald | df | Sig. | Exp(B) |
|---|---|---|---|---|---|---|---|
| Step | ankk1.84 | | | 4.420 | 2 | .110 | |
| 1^{a} | ankk1.84(1) | 1.748 | .879 | 3.949 | 1 | .047 | 5.741 |
| | ankk1.84(2) | 1.851 | .895 | 4.277 | 1 | .039 | 6.363 |
| | m1tsp45i | | | 5.796 | 2 | .055 | |
| | m1tsp45i(1) | .429 | .606 | .501 | 1 | .479 | 1.535 |
| | m1tsp45i(2) | 1.338 | .622 | 4.633 | 1 | .031 | 3.813 |
| | a1a4155 | | | 1.438 | 2 | .487 | |
| | a1a4155(1) | −.792 | .679 | 1.362 | 1 | .243 | .453 |
| | a1a4155(2) | −.217 | .523 | .172 | 1 | .678 | .805 |
| | Constant | −2.080 | 1.011 | 4.233 | 1 | .040 | .125 |

^{a}Variable(s) entered on step 1: ankk1.84, m1tsp45i, a1a4155.

LoR=[1−(−2.08+1.748A1+1.851A2+0.429B1+1.338B2−0.792C1−0.217C2)]

### Specificity=58.0%

### Sensitivity=70.0%

Positive predictive value (PPV)=62.5%
Negative predictive value (NPV)=65.9%
whereas:
A1=ANKK1 rs3897584−8882 C/C
A2=ANKK1 rs13897584−8882 C/G
B1=Muscarinic receptor M1 rs12295208−12064 T/T
B2=Muscarinic receptor M1 rs12295208−12064 T/C
C1=Alpha-1A adrenergic receptor rs2644627−4155 C/C
C2=Alpha-1A adrenergic receptor rs2644627−4155 C/G

### Olanzapine Algorithm for the Prediction of Improvement in Negative Symptoms (as Measured by PANSS Negative Scale)

TABLE 17
Classification Table^{a}

| | | | Predicted | | |
|---|---|---|---|---|---|
| | | | npanssRNR | | Per- |
| | | | Re- | Non-Re- | centage |
| | Observed | | sponder | sponder | Correct |
| Step | npanssRNR | Responder | 6 | 13 | 31.6 |
| 1 | | Non- | 3 | 62 | 95.4 |
| | | Responder | | | |
| | Overall Percentage | | | | 81.0 |

^{a}The cut value is .500

TABLE 18
Variables in the Equation

| | | B | S.E. | Wald | df | Sig. | Exp(B) |
|---|---|---|---|---|---|---|---|
| Step | Rs_1126422 | | | 2.318 | 2 | .314 | |
| 1^{a} | Rs_1126422(1) | −.240 | .796 | .091 | 1 | .763 | .787 |
| | Rs_1126422(2) | .764 | .882 | .750 | 1 | .387 | 2.147 |
| | Rs7702361 | | | 1.097 | 2 | .578 | |
| | Rs7702361(1) | .308 | .742 | .172 | 1 | .678 | 1.360 |
| | Rs7702361(2) | −.482 | .653 | .544 | 1 | .461 | .618 |
| | chatpp2(1) | 1.812 | .644 | 7.917 | 1 | .005 | 6.123 |
| | Constant | −.221 | .943 | .055 | 1 | .815 | .802 |

^{a}Variable(s) entered on step 1: Rs_1126422, Rs7702361, chatpp2.

LoR=[1−(−0.221−0.24A1+0.764A2+0.308B1−0.482B21.812C1)]

### Specificity=95.4%

### Sensitivity=31.6%

Positive predictive value (PPV)=66.7%
Negative predictive value (NPV)=82.7%
whereas:
A1=RXFP3 rs7702361−903 A/A
A2=RXFP3 rs7702361−903 C/C
B1=RXFP4 rs11264422−3678 A/A
B2=RXFP4 rs11264422−3678 A/T
C1=Choline Acetyltransferase ChAT rs8178984 C/C

### Olanzapine Algorithm for the Prediction of Improvement in General Psychopathology (as Measured by PANSS General Psychopathology Scale)

TABLE 19
Classification Table^{a}

| | | | Predicted | | |
|---|---|---|---|---|---|
| | | | pgpanssRNR | | Per- |
| | | | Re- | Non-Re- | centage |
| | Observed | | sponder | sponder | Correct |
| Step | pgpanssRNR | Responder | 17 | 13 | 56.7 |
| 1 | | Non- | 7 | 43 | 86.0 |
| | | Responder | | | |
| | Overall Percentage | | | | 75.0 |

^{a}The cut value is .500

TABLE 20
Variables in the Equation

| | | B | S.E. | Wald | df | Sig. | Exp(B) |
|---|---|---|---|---|---|---|---|
| Step | chatpp2(1) | 1.181 | .722 | 2.679 | 1 | .102 | 3.258 |
| 1^{a} | chatch6 | | | 5.081 | 2 | .079 | |
| | chatch6(1) | 1.690 | .789 | 4.588 | 1 | .032 | 5.418 |
| | chatch6(2) | .658 | .733 | .806 | 1 | .369 | 1.932 |
| | m1tsp45i | | | 7.587 | 2 | .023 | |
| | m1tsp45i(1) | .902 | .703 | 1.648 | 1 | .199 | 2.466 |
| | m1tsp45i(2) | 2.189 | .819 | 7.141 | 1 | .008 | 8.927 |
| | ankk1.84 | | | 2.301 | 2 | .317 | |
| | ankk1.84(1) | .657 | .918 | .512 | 1 | .474 | 1.929 |
| | ankk1.84(2) | 1.246 | .909 | 1.881 | 1 | .170 | 3.478 |
| | Constant | −3.461 | 1.355 | 6.528 | 1 | .011 | .031 |

^{a}Variable(s) entered on step 1: chatpp2, chatch6, m1tsp45i, ankk1.84.

LoR=[1−(−3.461+1.181A1+1.69B1+0.658B2+0.902C1+2.189C2+0.657D1+1.246D2)]

### Specificity=86.0%

### Sensitivity=56.7%

Positive predictive value (PPV)=70.8%
Negative predictive value (NPV)=76.8%
whereas:
A1=Choline Acetyltransferase ChAT rs8178984 C/C
B1=Choline Acetyltransferase ChAT rs1880676 G/G
B2=Choline Acetyltransferase ChAT rs1880676 G/A
C1=Muscarinic receptor M1 rs12295208−12064 T/T
C2=Muscarinic receptor M1 rs12295208−12064 T/C
D1=ANKK1 rs3897584−8882 C/C
D2=ANKK1 rs3897584−8882 C/G

SLC6A4 solute carrier family 6
(neurotransmitter transporter, serotonin),
member 4 (5-HTT)
LOCUS NM_001045 2775 bp mRNA linear PRI
09-DEC-2007
DEFINITION Homo sapiens solute carrier family 6
(neurotransmitter transporter, serotonin),
member 4 (SLC6A4), mRNA.
ACCESSION NM_001045
VERSION NM_001045.3 GI:145553964
>/tmp/readseq.in.15790 [Unknown form], 2775 bases,
905 checksum.

SEQ ID NO:20

acagccagcgccgccgggtgcctcgagggcgcgaggccagcccgcctgcc
cagcccgggaccagcctccccgcgcagcctggcaggtctcctggaggcaa
ggcgaccttgcttgccctctcttgcagaataacaaggggcttagccacag
gagttgctggcaagtggaaagaagaacaaatgagtcaatcccgacgtgtc
aatcccgacgatagagagctcggaggtgatccacaaatccaagcacccag
agatcaattgggatccttggcagatggacatcagtgtcatttactaacca
gcaggatggagacgacgcccttgaattctcagaagcagctatcagcgtgt
gaagatggagaagattgtcaggaaaacggagttctacagaaggttgttcc
caccccaggggacaaagtggagtccgggcaaatatccaatgggtactcag
cagttccaagtcctggtgcgggagatgacacacggcactctatcccagcg
accaccaccaccctagtggctgagcttcatcaaggggaacgggagacctg
gggcaagaaggtggatttccttctctcagtgattggctatgctgtggacc
tgggcaatgtctggcgcttcccctacatatgttaccagaatggagggggg
gcattcctcctcccctacaccatcatggccatttttgggggaatcccgct
cttttacatggagctcgcactgggacagtaccaccgaaatggatgcattt
caatatggaggaaaatctgcccgattttcaaagggattggttatgccatc
tgcatcattgccttttacattgcttcctactacaacaccatcatggcctg
ggcgctatactacctcatctcctccttcacggaccagctgccctggacca
gctgcaagaactcctggaacactggcaactgcaccaattacttctccgag
gacaacatcacctggaccctccattccacgtcccctgctgaagaatttta
cacgcgccacgtcctgcagatccaccggtctaaggggctccaggacctgg
ggggcatcagctggcagctggccctctgcatcatgctgatcttcactgtt
atctacttcagcatctggaaaggcgtcaagacctctggcaaggtggtgtg
ggtgacagccaccttcccttatatcatcctttctgtcctgctggtgaggg
gtgccaccctccctggagcctggaggggtgttctcttctacttgaaaccc
aattggcagaaactcctggagacaggggtgtggatagatgcagccgctca
gatcttcttctctcttggtccgggctttggggtcctgctggcttttgcta
gctacaacaagttcaacaacaactgctaccaagatgccctggtgaccagc
gtggtgaactgcatgacgagcttcgtttcgggatttgtcatcttcacagt
gctcggttacatggctgagatgaggaatgaagatgtgtctgaggtggcca
aagacgcaggtcccagcctcctcttcatcacgtatgcagaagcgatagcc
aacatgccagcgtccactttctttgccatcatcttctttctgatgttaat
cacgctgggcttggacagcacgtttgcaggcttggagggggtgatcacgg
ctgtgctggatgagttcccacacgtctgggccaagcgccgggagcggttc
gtgctcgccgtggtcatcacctgcttctttggatccctggtcaccctgac
ttttggaggggcctacgtggtgaagctgctggaggagtatgccacggggc
ccgcagtgctcactgtcgcgctgatcgaagcagtcgctgtgtcttggttc
tatggcatcactcagttctgcagggacgtgaaggaaatgctcggcttcag
cccggggtggttctggaggatctgctgggtggccatcagccctctgtttc
tcctgttcatcatttgcagttttctgatgagcccgccacaactacgactt
ttccaatataattatccttactggagtatcatcttgggttactgcatagg
aacctcatctttcatttgcatccccacatatatagcttatcggttgatca
tcactccagggacatttaaagagcgtattattaaaagtattaccccagaa
acaccaacagaaattccttgtggggacatccgcttgaatgctgtgtaaca
cactcaccgagaggaaaaaggcttctccacaacctcctcctccagttctg
atgaggcacgcctgccttctcccctccaagtgaatgagtttccagctaag
cctgatgatggaagggccttctccacagggacacagtctggtgcccagac
tcaaggcctccagccacttatttccatggattcccctggacatattccca
tggtagactgtgacacagctgagctggcctattttggacgtgtgaggatg
tggatggaggtgatgaaaaccaccctatcatcagttaggattaggtttag
aatcaagtctgtgaaagtctcctgtatcatttcttggtatgatcattggt
atctgatatctgtttgcttctaaaggtttcactgttcatgaatacgtaaa
ctgcgtaggagagaacagggatgctatctcgctagccatatattttctga
gtagcatatataattttattgctggaatctactagaaccttctaatccat
gtgctgctgtggcatcaggaaaggaagatgtaagaagctaaaatgaaaaa
tagtgtgtccatgcaaaaaaaaaaa
(alpha 1A) ADRA1A adrenergic, alpha-1A-, receptor
LOCUS NM_033303 2304 bp mRNA linear PRI
03-DEC-2007
DEFINITION Homo sapiens adrenergic, alpha-1A-,
receptor (ADRA1A), transcript variant 2, mRNA.
ACCESSION NM_033303
VERSION NM_033303.3 GI:111118985
>/tmp/readseq.in.15769 [Unknown form], 2304 bases,
1FE5 checksum.

SEQ ID NO:21

gaattccgaatcatgtgcagaatgctgaatcttcccccagccaggacgaa
taagacagcgcggaaaagcagattctcgtaattctggaattgcatgttgc
aaggagtctcctggatcttcgcacccagcttcgggtagggagggagtccg
ggtcccgggctaggccagcccggcaggtggagagggtccccggcagcccc
gcgcgcccctggccatgtctttaatgccctgccccttcatgtggccttct
gagggttcccagggctggccagggttgtttcccacccgcgcgcgcgctct
cacccccagccaaacccacctggcagggctccctccagccgagacctttt
gattcccggctcccgcgctcccgcctccgcgccagcccgggaggtggccc
tggacagccggacctcgcccggccccggctgggaccatggtgtttctctc
gggaaatgcttccgacagctccaactgcacccaaccgccggcaccggtga
acatttccaaggccattctgctcggggtgatcttggggggcctcattctt
ttcggggtgctgggtaacatcctagtgatcctctccgtagcctgtcaccg
acacctgcactcagtcacgcactactacatcgtcaacctggcggtggccg
acctcctgctcacctccacggtgctgcccttctccgccatcttcgaggtc
ctaggctactgggccttcggcagggtcttctgcaacatctgggcggcagt
ggatgtgctgtgctgcaccgcgtccatcatgggcctctgcatcatctcca
tcgaccgctacatcggcgtgagctacccgctgcgctacccaaccatcgtc
acccagaggaggggtctcatggctctgctctgcgtctgggcactctccct
ggtcatatccattggacccctgttcggctggaggcagccggcccccgagg
acgagaccatctgccagatcaacgaggagccgggctacgtgctcttctca
gcgctgggctccttctacctgcctctggccatcatcctggtcatgtactg
ccgcgtctacgtggtggccaagagggagagccggggcctcaagtctggcc
tcaagaccgacaagtcggactcggagcaagtgacgctccgcatccatcgg
aaaaacgccccggcaggaggcagcgggatggccagcgccaagaccaagac
gcacttctcagtgaggctcctcaagttctcccgggagaagaaagcggcca
aaacgctgggcatcgtggtcggctgcttcgtcctctgctggctgcctttt
ttcttagtcatgcccattgggtctttcttccctgatttcaagccctctga
aacagtttttaaaatagtattttggctcggatatctaaacagctgcatca
accccatcatatacccatgctccagccaagagttcaaaaaggcctttcag
aatgtcttgagaatccagtgtctctgcagaaagcagtcttccaaacatgc
cctgggctacaccctgcacccgcccagccaggccgtggaagggcaacaca
aggacatggtgcgcatccccgtgggatcaagagagaccttctacaggatc
tccaagacggatggcgtttgtgaatggaaatttttctcttccatgccccg
tggatctgccaggattacagtgtccaaagaccaatcctcctgtaccacag
cccggacgaagtctcgctctgtcaccaggctggagtgcagtggcatgatc
ttggctcactgcaacctccgcctcccgggttcaagagattctcctgcctc
agcctcccaagcagctgggactacagggatgtgccaccaggccgacgcca
ccaggcccagctaatttttgtatttttagtagagacggggtttcaccatg
ttggccaggatgatctcgatctcttgacctcatgatctgcctgcctcagc
ctcccaaagtgctgggattacaggcgtgagccaccgtgcccggcccaact
attttttttttttatcttttttaacagtgcaatcctttctgtggatgaaa
tcttgctcagaagctcaatatgcaaaagaaagaaaaacagcagggctgga
cggatgttgggagtggggtaagaccccaaccactcagaaccaccccccca
acacacacacacattctctccatggtgactggtgaggggcctctagaggg
tacatagtacaccatggagcacggtttaagcaccactggactacacattc
ttctgtggcagttatcttaccttcccatagacacccagcccatagccatt
ggtt
“ >/tmp/readseq.in.16991 [Unknown form], 476
bases, 196E checksum.
MVFLSGNASDSSNCTQPPAPVNISKAILLGVILGGLILFGVLGNILVILS
VACHRHLHSVTHYYIVNLAVADLLLTSTVLPFSAIFEVLGYWAFGRVFCN
IWAAVDVLCCTASIMGLCIISIDRYIGVSYPLRYPTIVTQRRGLMALLCV
WALSLVISIGPLFGWRQPAPEDETICQINEEPGYVLFSALGSFYLPLAII
LVMYCRVYVVAKRESRGLKSGLKTDKSDSEQVTLRIHRKNAPAGGSGMAS
AKTKTHFSVRLLKFSREKKAAKTLGIVVGCFVLCWLPFFLVMPIGSFFPD
FKPSETVFKIVFWLGYLNSCINPIIYPCSSQEFKKAFQNVLRIQCLCRKQ
SSKHALGYTLHPPSQAVEGQHKDMVRIPVGSRETFYRISKTDGVCEWKFF
SSMPRGSARITVSKDQSSCTTARTKSRSVTRLECSGMILAHCNLRLPGSR
DSPASASQAAGTTGMCHQADATRPS”
(M1) CHRM1: cholinergic receptor, muscarinic 1:
NM_000738 2863 bp mRNA linear PRI 25-SEP-2007
DEFINITION Homo sapiens cholinergic receptor,
muscarinic 1 (CHRM1), mRNA.
ACCESSION NM_000738
VERSION NM_000738.2 GI:37622909
>/tmp/readseq.in.15796 [Unknown form], 2863 bases,
A5 checksum.

SEQ ID NO:28

tggggctcaaattgggtgccctggtgaaggaggggggcacactccagaac
ctagtccaaccccagacgctgcctgaggcttccctccagctcccctccct
tccttttctccctttcctccctccctctctttccctttctccctccccgc
taaggctggcgtgccagggggtgggacatgccaatcactggctgtgcctc
tcccgctgccagcacagggcgcagctccccctgggagccaggtgtttggg
tccctggagacgccgcaggcccccagggaggcagtggggctgaggaccct
acagacccctcttcagccccgtggtgatgactttcccctgaggaagccct
gtagcgtgcctggaggaaggggctctccaaccccagccccacctagccac
catgaacacttcagccccacctgctgtcagccccaacatcaccgtcctgg
caccaggaaagggtccctggcaagtggccttcattgggatcaccacgggc
ctcctgtcgctagccacagtgacaggcaacctgctggtactcatctcttt
caaggtcaacacggagctcaagacagtcaataactacttcctgctgagcc
tggcctgtgctgacctcatcatcggtaccttctccatgaacctctatacc
acgtacctgctcatgggccactgggctctgggcacgctggcttgtgacct
ctggctggccctggactatgtggccagcaatgcctccgtcatgaatctgc
tgctcatcagctttgaccgctacttctccgtgactcggcccctgagctac
cgtgccaagcgcacaccccgccgggcagctctgatgatcggcctggcctg
gctggtttcctttgtgctctgggccccagccatcctcttctggcagtacc
tggtaggggagcggacagtgctagctgggcagtgctacatccagttcctc
tcccagcccatcatcacctttggcacagccatggctgccttctacctccc
tgtcacagtcatgtgcacgctctactggcgcatctaccgggagacagaga
accgagcacgggagctggcagcccttcagggctccgagacgccaggcaaa
gggggtggcagcagcagcagctcagagaggtctcagccaggggctgaggg
ctcaccagagactcctccaggccgctgctgtcgctgctgccgggccccca
ggctgctgcaggcctacagctggaaggaagaagaggaagaggacgaaggc
tccatggagtccctcacatcctcagagggagaggagcctggctccgaagt
ggtgatcaagatgccaatggtggaccccgaggcacaggcccccaccaagc
agcccccacggagctccccaaatacagtcaagaggccgactaagaaaggg
cgtgatcgagctggcaagggccagaagccccgtggaaaggagcagctggc
caagcggaagaccttctcgctggtcaaggagaagaaggcggctcggaccc
tgagtgccatcctcctggccttcatcctcacctggacaccgtacaacatc
atggtgctggtgtccaccttctgcaaggactgtgttcccgagaccctgtg
ggagctgggctactggctgtgctacgtcaacagcaccatcaaccccatgt
gctacgcactctgcaacaaagccttccgggacacctttcgcctgctgctg
ctttgccgctgggacaagagacgctggcgcaagatccccaagcgccctgg
ctccgtgcaccgcactccctcccgccaatgctgatagccccctctcctgc
atccctccaccccagtccccgggaaaggccggtgggaagagggcaggggc
tgcatcctcagccccagggccctgctcaggcctcacctggcttcccagga
ccctgggtcaccttcctgggcagcccagagagaccctgccaactttccag
acttcgctattcccaggcagggagggaaacccggggaactggtttttctg
ttccctgctgggtgggaatgcgctcttcaccaggaagaaggcccgggagg
aggatccgggctttggactccttgtttgcctttaggcaggaagtcaggag
ccagcagggcgggccaggagaaagaaggcttaacattaagtattccttgg
cccagcagcggcccagattgcggtgtgagatggtgccccctggggggcac
agccagaaactgaactggccgctgggagaaaagccagatgacagggagct
ggggaatcccctcgcttcataggcagagcccgcccacctgggccctaggc
atactctccaggattgtccacaaatgtcctcagagggtccctaggtgggt
caactccaaggcaaatgtccaagcatcagcaagacaatgacactggaagg
gtccggcttggctagtcacatatcaagtcccgaggcagcaacaggaccag
gagccaggtgtcctgactgtcctacaatatcattttcctgggagtgggag
tcaagtgtgcctgctatccagccgcaaatccataccccctgccccagaga
agcctcagtccctccctcctggctcacagccaccacctggatggatctgc
tccatgcagatctagccaggcctcccgcatgctgcctgcctccggccctg
ccccacacaggcctggcccagccagcaggttctctcctgtgagctcccca
atccaacccatgcatggcctcccagccacccggatctccaggcccagcct
ggccccaaatgttctttcctttcatcctcagcaagtgctgagtctgtgaa
taaagccacataaccagcgggcaaaaaaaaaaaaaaaaaaaaaaaaaaaa
aaaaaaaaaaaaa
CHAT choline acetyltransferase
NM_020549 2485 bp mRNA linear PRI 09-DEC-2007
DEFINITION Homo sapiens choline acetyltransferase
(CHAT), transcript variant M, mRNA.
ACCESSION NM_020549
VERSION NM_020549.3 GI:119433674
KEYWORDS
>/tmp/readseq.in.15802 [Unknown form], 2485 bases,
F41 checksum.

SEQ ID NO:29

tggggttggggaagtgcggtgactgggaaatgctgagctaggggcaggag
gcatgggcgggacagtgttctgtgcccccttctagagcctaaatttgttg
cccgagttcctccgggaagcgctccgggtagattctgggggccgggagct
gagatccctgggcggggagctggggaagggatggggctgaggacagcgaa
gaagagggggcttgggggaggggggaaatggaagagagaggagggaggag
gtacaagaggaaggagagaagtgcggccagcttgctttctccagtcgggt
ggccgcggggacccgggcgacgtcggaggccctgccgggaacccaggctg
cagcccccacccccgcgctgcgacacgccccccaccccttccggctcaca
cccccgcccacactcctgagtggtgcggtgcagcgtcggccgaggcagca
gagccgaggagagcaggtccacacctctgcatccctgcaccaggactcac
caagacgcccatcctggaaaaggtcccccgtaagatggcagcaaaaactc
ccagcagtgaggagtctgggctgcccaaactgcccgtgcccccgctgcag
cagaccctggccacgtacctgcagtgcatgcgacacttggtgtctgagga
gcagttcaggaagagccaggccattgtgcagcagtttggggcccctggtg
gcctcggcgagaccctgcagcagaaactcctggagcggcaggagaagaca
gccaactgggtgtctgagtactggctgaatgacatgtatctcaacaaccg
cctggccctgcctgtcaactccagccctgccgtgatctttgctcggcagc
acttccctggcaccgatgaccagctgaggtttgcagccagcctcatctct
ggtgtactcagctacaaggccctgctggacagccactccattcccactga
ctgtgccaaaggccagctgtcagggcagcccctttgcatgaagcaatact
atgggctcttctcctcctaccggctccccggccatacccaggacacgctg
gtggctcagaacagcagcatcatgccggagcctgagcacgtcatcgtagc
ctgctgcaatcagttctttgtcttggatgttgtcattaatttccgccgtc
tcagtgagggggatctgttcactcagttgagaaagatagtcaaaatggct
tccaacgaggacgagcgtttgcctccaattggcctgctgacgtctgacgg
gaggagcgagtgggccgaggccaggacggtcctcgtgaaagactccacca
accgggactcgctggacatgattgagcgctgcatctgccttgtatgcctg
gacgcgccaggaggcgtggagctcagcgacacccacagggcactccagct
ccttcacggcggaggctacagcaagaacggggccaatcgctggtacgaca
agtccctgcagtttgtggtgggccgagacggcacctgcggtgtggtgtgc
gaacactccccattcgatggcatcgtcctggtgcagtgcactgagcatct
gctcaagcacgtgacgcagagcagcaggaagctgatccgagcagactccg
tcagcgagctccccgccccccggaggctgcggtggaaatgctccccggaa
attcaaggccacttagcctcctcggcagaaaaacttcaacgaatagtaaa
gaaccttgacttcattgtctataagtttgacaactatgggaaaacattca
ttaagaagcagaaatgcagccctgatgccttcatccaggtggccctccag
ctggccttctacaggctccatcgaagactggtgcccacctacgagagcgc
gtccatccgccgattccaggagggacgcgtggacaacatcagatcggcca
ctccagaggcactggcttttgtgagagccgtgactgaccacaaggctgct
gtgccagcttctgagaagcttctgctcctgaaggatgccatccgtgccca
gactgcatacacagtcatggccataacagggatggccattgacaaccacc
tgctggcactgcgggagctggcccgggccatgtgcaaggagctgcccgag
atgttcatggatgaaacctacctgatgagcaaccggtttgtcctctccac
tagccaggtgcccacaaccacggagatgttctgctgctatggtcctgtgg
tcccaaatgggtatggtgcctgctacaacccccagccagagaccatcctt
ttctgcatctctagctttcacagctgcaaagagacttcttctagcaagtt
tgcaaaagctgtggaagaaagcctcattgacatgagagacctctgcagtc
tgctgccgcctactgagagcaagccattggcaacaaaggaaaaagccacg
aggcccagccagggacaccaaccttgactcctgccactaggtttcacctc
ccaaacccagcctctagaacagccagaccctgcag
NRG1: neuregulin 1: Neuregulin 1 (NRG1)
HUMGGFB 1199 bp mRNA linear PRI 12-JUN-1993
DEFINITION Human recombinant glial growth factor
mRNA, complete cds and flanking region.
ACCESSION L12261
VERSION L12261.I GI:292049
KEYWORDS glial growth factor; neuregulin.
SOURCE Homo sapiens (human)
//
>/tmp/readseq.in.15805 [Unknown form], 1199 bases,
209A checksum.

SEQ ID NO:30

gcgcggaggccaggagctgagcggcggcggctgccggacgatgggagcgt
gagcaggacggtgataacctctccccgatcgggttgcgagggcgccgggc
agaggccaggacgcgagccgccagcggcgggacccatcgacgacttcccg
gggcgacaggagcagccccgagagccagggcgagcgcccgttccaggtgg
ccggaccgcccgccgcgtccgcgccgcgctccctgcaggcaacgggagac
gcccccgcgcagcgcgagcgcctcagcgcggccgctcgctctccccatcg
agggacaaacttttcccaaacccgatccgagcccttggaccaaactcgcc
tgcgccgagagccgtccgcgtagagcgctccgtctccggcgagatgtccg
agcgcaaagaaggcagaggcaaagggaagggcaagaagaaggagcgaggc
tccggcaagaagccggagtccgcggcgggcagccagagcccagccttgcc
tccccgattgaaagagatgaaaagccaggaatcggctgcaggttccaaac
tagtccttcggtgtgaaaccagttctgaatactcctctctcagattcaag
tggttcaagaatgggaatgaattgaatcgaaaaaacaaaccacaaaatat
caagatacaaaaaaagccagggaagtcagaacttcgcattaacaaagcat
cactggctgattctggagagtatatgtgcaaagtgatcagcaaattagga
aatgacagtgcctctgccaatatcaccatcgtggaatcaaacgagatcat
cactggtatgccagcctcaactgaaggagcatatgtgtcttcagagtctc
ccattagaatatcagtatccacagaaggagcaaatacttcttcatctaca
tctacatccaccactgggacaagccatcttgtaaaatgtgcggagaagga
gaaaactttctgtgtgaatggaggggagtgcttcatggtgaaagaccttt
caaacccctcgagatacttgtgcaagtgcccaaatgagtttactggtgat
cgctgccaaaactacgtaatggccagcttctacagtacgtccactccctt
tctgtctctgcctgaataggagcatgctcagttggtgctgctttcttgtt
gctgcatctcccctcagattccacctagagctagatgtgtcttaccaga
ADRA2A
NM_000681 3653 bp mRNA linear PRI 26-SEP-2007
DEFINITION Homo sapiens adrenergic, alpha-2A-,
receptor (ADRA2A), mRNA.
ACCESSION NM_000681
VERSION NM_000681.2 GI:15718669
KEYWORDS
>/tmp/readseq.in.15814 [Unknown form], 3653 bases,
13EE checksum.

SEQ ID NO:26

gcgctcggcgcccaccaggcggacgcccaggagaacccctgcctccgtcg
cggctcctggagagctgatcgttcacctgccccggcccgcctgaggacgg
gggtgccttcatgcggcccccacactcctcaccccgccgccgccgccgtc
cccgagctccgcacagtgcgccccagccccagcagggcgcacaactttgg
aagtctcgcggcgctccgagaggcggcagagtccgcgccccagccccggg
ccgggccgggccagaaccgcagcgtctgggggaagccagagagtcggtaa
tcgcttcggggatgtaaggcgacagacataggacccccgagctcgcatca
gcacccttcggctgcctcccggggtgggggcgggccccgcacacggtaag
acctcttgctttcgctcaggctcaagattcaagatacagatattgatatg
tatatatatatttaatttcctgtcatccttccaagttatcaggccaccga
tgatttttgttctcccttcttgaagaataaatctctctttacccatcggc
tctccctactctctcccgccgcttagaaataaaacttggctgtattagga
gctcggagcaagaaggcgcccaccgagagcgtctgaagcgcgagccaggc
gcagttcgcgggacccgggccatgggccgctagcggtcctccagttcggg
cccggcctccctgcggccccctccctatgtgagccgcagccaggcgagcg
gggcgccggaggaagaggaggacccacgggcgccgggccggaaggcagct
ggcagcaggcccaggccagcgggcgcccgcgttcatgttccgccaggagc
agccgttggccgagggcagctttgcgcccatgggctccctgcagccggac
gcgggcaacgcgagctggaacgggaccgaggcgccggggggcggcgcccg
ggccaccccttactccctgcaggtgacgctgacgctggtgtgcctggccg
gcctgctcatgctgctcaccgtgttcggcaacgtgctcgtcatcatcgcc
gtgttcacgagccgcgcgctcaaggcgccccaaaacctcttcctggtgtc
tctggcctcggccgacatcctggtggccacgctcgtcatccctttctcgc
tggccaacgaggtcatgggctactggtacttcggcaaggcttggtgcgag
atctacctggcgctcgacgtgctcttctgcacgtcgtccatcgtgcacct
gtgcgccatcagcctggaccgctactggtccatcacacaggccatcgagt
acaacctgaagcgcacgccgcgccgcatcaaggccatcatcatcaccgtg
tgggtcatctcggccgtcatctccttcccgccgctcatctccatcgagaa
gaagggcggcggcggcggcccgcagccggccgagccgcgctgcgagatca
acgaccagaagtggtacgtcatctcgtcgtgcatcggctccttcttcgct
ccctgcctcatcatgatcctggtctacgtgcgcatctaccagatcgccaa
gcgtcgcacccgcgtgccacccagccgccggggtccggacgccgtcgccg
cgccgccggggggcaccgagcgcaggcccaacggtctgggccccgagcgc
agcgcgggcccggggggcgcagaggccgaaccgctgcccacccagctcaa
cggcgcccctggcgagcccgcgccggccgggccgcgcgacaccgacgcgc
tggacctggaggagagctcgtcttccgaccacgccgagcggcctccaggg
ccccgcagacccgagcgcggtccccggggcaaaggcaaggcccgagcgag
ccaggtgaagccgggcgacagcctgccgcggcgcgggccgggggcgacgg
ggatcgggacgccggctgcagggccgggggaggagcgcgtcggggctgcc
aaggcgtcgcgctggcgcgggcggcagaaccgcgagaagcgcttcacgtt
cgtgctggccgtggtcatcggagtgttcgtggtgtgctggttccccttct
tcttcacctacacgctcacggccgtcgggtgctccgtgccacgcacgctc
ttcaaattcttcttctggttcggctactgcaacagctcgttgaacccggt
catctacaccatcttcaaccacgatttccgccgcgccttcaagaagatcc
tctgtcggggggacaggaagcggatcgtgtgaggtttccgctggcgcccg
cgtagactcacgctgactgcaggcagcggggggcatcgaggggtgcttag
ccccagggcactcagaaacccgggcgctgcctgctctgcgtttcctcgtc
tggggtggctctgcagcctcctgcgggcgggcgtctgctgctcctacaag
ggaagcttcttgctgccaggcccacacatccccagttgttggtttggcca
ctcttgacctggagccatcttcctagtgggccacccctaatcactattgc
ttcctaaaggtattttcaccctcttcgcctggtacagccctcacagctct
tcagagcaagcactggactacaagggcatggctcacaaaaggttaatgga
tgggggttacctagccctggctaattccccttccattcccaactctctct
ctctttttgaagaaaaatgctaagggcagccctgcctgccctccccatcc
cccgctgtaaatatacactatttttgatagcacacatggggcccccatat
ctcttggccttggttttgatgttgaaatcctggccttgggagagatgcct
tccaggcagacacagctgtctggttcaggccaagcccctttgcaatgcaa
gccctttctggtgttatgaagtccctctatgtcgtcgttttcaccagcaa
ctggtgactgtcccttcgacacggacctgctttgagatttcctgacaggg
aaaagatttctgtccatttttttcctgtgcctaacagcataattgccttt
tcctatgtaaatattatgatggtggatcaagacataagtaaatgagcctt
tctgcctcacatcagccctgtgtataaagccattattctctgatgcactg
tttgccccagtaactcactttaaaacctctctttccagtgttccctctct
ccctccagggccactgcttgaagaagaatatgtatgtttctatcttgtat
gtctgtgtgcccctcctgccccgaaagtgctgactatggggaaatctttt
agctgctgtttttagactccaaggagtggaaattatgtggaagaagcaaa
cctgatacaatttgcccaaggtaaacagtttgaaaagacaaatgggcctg
ccaaactgtacagtttcttccccaagagctgttaggtatcaaaatgttgt
cctttcccccctccgtgcttttctggttgagatcatgtcattgatgaact
gccaaagtcaggggaggagggcagagactttgtgtttacatctgcatttc
tacatgttttagacagagacaatttaaggcctgcactcttatttcactaa
agaaaaactaatgtcagcacatgttgctaatgacagtggattttttttta
aataaaaaagtttacagatcaaatgtgaaataaatatgaatggagtggtc
aaa
ANKK1
NM_178510 2543 bp mRNA linear PRI 18-NOV-2007
DEFINITION Homo sapiens ankyrin repeat and kinase
domain containing 1 (ANKK1), mRNA.
ACCESSION NM_178510
VERSION NM_178510.1 GI:30425443
>/tmp/readseq.in.16905 [Unknown form], 2543 bases,
AB1 checksum.

SEQ ID NO:35

cggacccgaggagcaggaagcggcggctccttcggccacccaggcagcag
ccacagcggggagtgcgcggcgcggggacaggaagagaggggcaatggct
gccgaccccaccgagctgcggctgggcagcctccccgtcttcacccgcga
cgacttcgagggcgactggcgcctagtggccagcggcggcttcagccagg
tgttccaggcgcggcacaggcgctggcggacggagtacgccatcaagtgc
gccccctgccttccacccgacgccgccagctctgatgtgaattacctcat
tgaagaagctgccaaaatgaagaagatcaagtttcagcacatcgtgtcta
tctacggggtgtgcaagcagcccctgggtattgtgatggagtttatggcc
aacggctccctggagaaggtgctgtccacccacagcctctgctggaagct
caggttccgcatcatccatgagaccagcttggccatgaacttcctgcaca
gcattaagccgcctctgctccacctggacctcaagccgggcaacatactc
ctggacagcaacatgcatgtcaaaatttcagacttcggcctgtccaagtg
gatggaacagtccacccggatgcagtacatcgagaggtcggctctgcggg
gcatgctcagctacatcccccctgagatgttcctggagagtaacaaggcc
ccaggacctaaatatgatgtgtacagctttgcaattgtcatctgggagct
actcactcagaagaaaccatactcagggttcaacatgatgatgattatta
tccgagtggcggcaggcatgcggccctccctacagcctgtctctgaccaa
tggccaagcgaggcccagcagatggtggacctgatgaaacgctgctggga
ccaggaccccaagaagaggccatgctttctagacattaccatcgagacag
acatactgctgtcactgctgcagagtcgtgtggcagtcccagagagcaag
gccctggccaggaaggtgtcctgcaagctgtcgctgcgccagcccgggga
ggttaatgaggacatcagccaggaactgatggacagtgactcaggaaact
acctgaagcgggcccttcagctctccgaccgtaagaatttggtcccgaga
gatgaggaactgtgtatctatgagaacaaggtcacccccctccacttcct
ggtggcccagggcagtgtggagcaggtgaggttgctgctggcccacgagg
tagacgtggactgccagacggcctccggatacacgcccctcctgatcgcc
gcccaggaccagcaacccgacctctgtgccctgcttttggcacatggtgc
tgatgccaaccgagtggatgaggatggctgggccccactgcactttgcag
cccagaatggggatgacggcactgcgcgcctgctcctggaccacggggcc
tgtgtggatgcccaggaacgtgaagggtggacccctcttcacctggctgc
acagaataactttgagaatgtggcacggcttctggtctcccgtcaggctg
accccaacctgcatgaggctgagggcaagacccccctccatgtggccgcc
tactttggccatgttagcctggtcaagctgctgaccagccagggggctga
gttggatgctcagcagagaaacctgagaacaccactgcacctggcagtag
agcggggcaaagtgagggccatccaacacctgctgaagagtggagcggtc
cctgatgcccttgaccagagcggctacggcccactgcacactgcagctgc
caggggcaaatacctgatctgcaagatgctgctcaggtacggagccagcc
ttgagctgcccacccaccagggctggacacccctgcatctagcagcctac
aagggccacctggagatcatccatctgctggcagagagccacgcaaacat
gggtgctcttggagctgtgaactggactcccctgcacctagctgcacgcc
acggggaggaggcggtggtgtcagcactgctgcagtgtggggctgacccc
aatgctgcagagcagtcaggctggacacccctccacctggcggtccagag
gagcaccttcctgagtgtcatcaacctcctagaacatcacgcaaatgtcc
acgcccgcaacaaggtgggctggacacccgcccacctggccgccctcaag
ggcaacacagccatcctcaaagtgctggtcgaggcaggcgcccagctgga
cgtccaggatggagtgagctgcacacccctgcaactggccctccgcagcc
gaaagcagggcatcatgtccttcctagagggcaaggagccgtcagtggcc
actctgggtggttctaagccaggagccgagatggaaatttagacaacttg
gccagccgtggtggctcacgtctgtaatcccagcactttgggaggctgag
gcaggcagatcacctgatatcaagagtttgaggccagcctggccaacatg
gcaaaaccctgtctctgctaaaaatacaaaatttagctgggta
//
DRD3
NM_033660 1193 bp mRNA linear PRI 27-NOV-2005
DEFINITION Homo sapiens dopamine receptor D3
(DRD3), transcript variant d, mRNA.
ACCESSION NM_033660
VERSION NM_033660.1 GI:16445399
>/tmp/readseq.in.16915 [Unknown form], 1193 bases,
9FE checksum.

SEQ ID NO:52

taaagaaaacggatacattcgaaagcagctatgaaacatgcactaaggtc
taatagggaagctggaaaagcagcactcaagtaatttcaccttagaggca
aaaatgggtgatttctttctgttcatttcatagtttctgagtcctgagaa
aggcaaagtttgctttgcttgggtatgtctgctgtcagtaaatggctgca
ggagccgaagtggtaaactcctcggtctccagaaatcagaagaaaatttt
aggaagccccttggcatcacgcacctccctctgggctatggcatctctga
gtcagctgagtagccacctgaactacacctgtggggcagagaactccaca
ggtgccagccaggcccgcccacatgcctactatgccctctcctactgcgc
gctcatcctggccatcgtcttcggcaatggcctggtgtgcatggctgtgc
tgaaggagcgggccctgcagactaccaccaactacttagtagtgagcctg
gctgtggcagacttgctggtggccaccttggtgatgccctgggtggtata
cctggaggtgacaggtggagtctggaatttcagccgcatttgctgtgatg
tttttgtcaccctggatgtcatgatgtgtacagccagcatccttaatctc
tgtgccatcagcatagacaggtacactgcagtggtcatgcccgttcacta
ccagcatggcacgggacagagctcctgtcggcgcgtggccctcatgatca
cggccgtctgggtactggcctttgctgtgtcctgccctcttctgtttggc
tttaataccacaggggaccccactgtctgctccatctccctgagtcccac
catagcgcccaagctcagcttagaagttcgaaaactcagcaatggcagat
tatcgacatctttgaagctggggcccctgcaacctcggggagtgccactt
cgggagaagaaggcaacccaaatggtggccattgtgcttggggccttcat
tgtctgctggctgcccttcttcttgacccatgttctcaatacccactgcc
agacatgccacgtgtccccagagctttacagtgccacgacatggctgggc
tacgtgaatagcgccctcaaccctgtgatctataccaccttcaatatcga
gttccggaaagccttcctcaagatcctgtcttgctgagggagc
HTR6
NM_000871 1984 bp mRNA linear PRI 17-SEP-2007
DEFINITION Homo sapiens 5-hydroxytryptamine
(serotonin) receptor 6 (HTR6), mRNA.
ACCESSION NM_000871
VERSION NM_000871.1 GI:4504544
>/tmp/readseq.in.16938 [Unknown form], 1984 bases,
1FC0 checksum.

SEQ ID NO:53

cccgagagcgcccattcacccccctcacccacctccccgcgttcccactt
ccccgcactctgacccggccggacgcccctcccctatcttgccgcccgcc
ccctccagggggctctgctcccaccccagggagcccatccgacctctgct
tgacttcccgccgcttccttcaggggcctcggctcatcgggtgcccctcc
ccaaacttccaacccgtttgctccaggagttcctgccccatccccgaggg
cgcccaaatagccacactgtgtcctcctgtagtcgccgccccctgaccta
gcgcgacccagcgcccccgcccatgtccccccactcacctcccccggggg
gcgtggtgagtcgcggtctgttctcacggacggtccccgtccagcctgcg
cttcgccggggccctcatctgctttcccgccaccctatcactcccttgcc
gtccaccctcggtcctcatggtcccagagccgggcccaaccgccaatagc
accccggcctggggggcagggccgccgtcggccccggggggcagcggctg
ggtggcggccgcgctgtgcgtggtcatcgcgctgacggcggcggccaact
cgctgctgatcgcgctcacctgcactcagcccgcgctgcgcaacacgtcc
aacttcttcctggtgtcgctcttcacgtctgacctgatggtggggctggt
ggtgatgccgccggccatgctgaacgcgctgtacgggcgctgggtgctgg
cgcgcggcctctgcctgctctggaccgccttcgacgtgatgtgctgcagc
gcctccatcctcaacctctgcctcatcagcctggaccgctacctgctcat
cctctcgccgctgcgctacaagctgcgcatgacgcccctgcgtgccctgg
ccctagtcctgggcgcctggagcctcgccgctctcgcctccttcctgccc
ctgctgctgggctggcacgagctgggccacgcacggccacccgtccctgg
ccagtgccgcctgctggccagcctgccttttgtccttgtggcgtcgggcc
tcaccttcttcctgccctcgggtgccatatgcttcacctactgcaggatc
ctgctagctgcccgcaagcaggccgtgcaggtggcctccctcaccaccgg
catggccagtcaggcctcggagacgctgcaggtgcccaggaccccacgcc
caggggtggagtctgctgacagcaggcgtctagccacgaagcacagcagg
aaggccctgaaggccagcctgacgctgggcatcctgctgggcatgttctt
tgtgacctggttgcccttctttgtggccaacatagtccaggccgtgtgcg
actgcatctccccaggcctcttcgatgtcctcacatggctgggttactgt
aacagcaccatgaaccccatcatctacccactcttcatgcgggacttcaa
gcgggcgctgggcaggttcctgccatgtccacgctgtccccgggagcgcc
aggccagcctggcctcgccatcactgcgcacctctcacagcggcccccgg
cccggccttagcctacagcaggtgctgccgctgcccctgccgccggactc
agattcggactcagacgcaggctcaggcggctcctcgggcctgcggctca
cggcccagctgctgcttcctggcgaggccacccaggaccccccgctgccc
accagggccgctgccgccgtcaatttcttcaacatcgaccccgcggagcc
cgagctgcggccgcatccacttggcatccccacgaactgacccgggcttg
gggctggccaatggggagctggattgagcagaacccagaccctgagtcct
tgggccagctcttggctaagaccaggaggctgcaagtctcctagaagccc
tctgagctccagaggggtgcgcagagctgaccccctgctgccatctccag
gccccttacctgcagggatcatagctgactcaga
//
RXFP3
NM_016568 1854 bp mRNA linear PRI 03-SEP-2007
DEFINITION Homo sapiens relaxin/insulin-like
family peptide receptor 3 (RXFP3), mRNA.
ACCESSION NM_016568
VERSION NM_016568.2 GI:141802857
>/tmp/readseq.in.16968 [Unknown form], 1854 bases,
267 checksum.

SEQ ID NO:54

ttggggagttatgcgccagtgccccagtgaccgcgggacacggagagggg
aagtctgcgttgtacataaggacctagggactccgagcttggcctgagaa
cccttggacgccgagtgcttgccttacgggctgcactcctcaactctgct
ccaaagcagccgctgagctcaactcctgcgtccagggcgttcgctgcgcg
ccaggacgcgcttagtacccagttcctgggctctctcttcagtagctgct
ttgaaagctcccacgcacgtcccgcaggctagcctggcaacaaaactggg
gtaaaccgtgttatcttaggtcttgtcccccagaacatgacctagaggta
cctgcgcatgcagatggccgatgcagccacgatagccaccatgaataagg
cagcaggcggggacaagctagcagaactcttcagtctggtcccggacctt
ctggaggcggccaacacgagtggtaacgcgtcgctgcagcttccggactt
gtggtgggagctggggctggagttgccggacggcgcgccgccaggacatc
ccccgggcagcggcggggcagagagcgcggacacagaggcccgggtgcgg
attctcatcagcgtggtgtactgggtggtgtgcgccctggggttggcggg
caacctgctggttctctacctgatgaagagcatgcagggctggcgcaagt
cctctatcaacctcttcgtcaccaacctggcgctgacggactttcagttt
gtgctcaccctgcccttctgggcggtggagaacgctcttgacttcaaatg
gcccttcggcaaggccatgtgtaagatcgtgtccatggtgacgtccatga
acatgtacgccagcgtgttcttcctcactgccatgagtgtgacgcgctac
cattcggtggcctcggctctgaagagccaccggacccgaggacacggccg
gggcgactgctgcggccggagcctgggggacagctgctgcttctcggcca
aggcgctgtgtgtgtggatctgggctttggccgcgctggcctcgctgccc
agtgccattttctccaccacggtcaaggtgatgggcgaggagctgtgcct
ggtgcgtttcccggacaagttgctgggccgcgacaggcagttctggctgg
gcctctaccactcgcagaaggtgctgttgggcttcgtgctgccgctgggc
atcattatcttgtgctacctgctgctggtgcgcttcatcgccgaccgccg
cgcggcggggaccaaaggaggggccgcggtagccggaggacgcccgaccg
gagccagcgcccggagactgtcgaaggtcaccaaatcagtgaccatcgtt
gtcctgtccttcttcctgtgttggctgcccaaccaggcgctcaccacctg
gagcatcctcatcaagttcaacgcggtgcccttcagccaggagtatttcc
tgtgccaggtatacgcgttccctgtgagcgtgtgcctagcgcactccaac
agctgcctcaaccccgtcctctactgcctcgtgcgccgcgagttccgcaa
ggcgctcaagagcctgctgtggcgcatcgcgtctccttcgatcaccagca
tgcgccccttcaccgccactaccaagccggagcacgaggatcaggggctg
caggccccggcgccgccccacgcggccgcggagccggacctgctctacta
cccacctggcgtcgtggtctacagcggggggcgctacgacctgctgccca
gcagctctgcctactgacgcaggcctcaggcccagggcgcgccgtcgggg
caaggtggccttccccgggcggtaaagaggtgaaaggatgaaggagggct
gggg
RXFP4
NM_181885 1146 bp mRNA linear PRI 03-SEP-2007
DEFINITION Homo sapiens relaxin/insulin-like
family peptide receptor 4 (RXFP4), mRNA.
ACCESSION NM_181885
VERSION NM_181885.2 GI:111548672
>/tmp/readseq.in.16974 [Unknown form], 1148 bases,
71F checksum.

SEQ ID NO:55

accaatctctgatgccctgcgatgcccacactcaatacttctgcctctcc
acccacattcttctgggccaatgcctccggaggcagtgtgctgagtgctg
atgatgctccgatgcctgtcaaattcctagccctgaggctcatggttgcc
ctggcctatgggcttgtgggggccattggcttgctgggaaatttggcggt
gctgtgggtactgagtaactgtgcccggagagcccctggcccaccttcag
acaccttcgtcttcaacctggctctggcggacctgggactggcactcact
ctccccttttgggcagccgagtcggcactggactttcactggcccttcgg
aggtgccctctgcaagatggttctgacggccactgtcctcaacgtctatg
ccagcatcttcctcatcacagcgctgagcgttgctcgctactgggtggtg
gccatggctgcggggccaggcacccacctctcactcttctgggcccgaat
agccaccctggcagtgtgggcggcggctgccctggtgacggtgcccacag
ctgtcttcggggtggagggtgaggtgtgtggtgtgcgcctttgcctgctg
cgtttccccagcaggtactggctgggggcctaccagctgcagagggtggt
gctggctttcatggtgcccttgggcgtcatcaccaccagctacctgctgc
tgctggccttcctgcagcggcggcaacggcggcggcaggacagcagggtc
gtggcccgctctgtccgcatcctggtggcttccttcttcctctgctggtt
tcccaaccatgtggtcactctctggggtgtcctggtgaagtttgacctgg
tgccctggaacagtactttctatactatccagacgtatgtcttccctgtc
actacttgcttggcacacagcaatagctgcctcaaccctgtgctgtactg
tctcctgaggcgggagccccggcaggctctggcaggcaccttcagggatc
tgcggttgaggctgtggccccagggcggaggctgggtgcaacaggtggcc
ctaaagcaggtaggcaggcggtgggtcgcaagcaacccccgggagagccg
cccttctaccctgctcaccaacctggacagagggacacccgggtga//

All publications mentioned in the present specification, and references cited in said publications, are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method of determining the likelihood of a response to olanzapine treatment in a patient comprising detecting the allelic forms of one or more polymorphisms in one or more genes of said patient, selected from the group consisting of: ADRA2A, ADRA1A and ANKK1.

2. A method of determining the likelihood of a response to olanzapine treatment in a patient comprising detecting the allelic forms of one or more polymorphisms in each of the following genes of said patient: ADRA2A, ADRA1A and ANKK1.

3. The method of claim 1, further comprising detecting the allelic forms of one or more polymorphisms in one or more of the following genes of said patient: D3, 5-HTT, M1 and 5-HT6.

4. The method of any of claim 2, further comprising detecting the allelic forms of one or more polymorphisms in each of the following genes of said patient: D3, 5-HTT, M1 and 5-HT6.

5. The method of claim 1 or 2 or 3 or 4, wherein said one or more polymorphisms in ADRA1A, comprises 4155-C/G , wherein said one or more polymorphisms in ADRA2A comprises −2211-A/T, and wherein said one or more polymorphisms in ANKK1 comprises −8882-C/G.

6. The method of claim 5, wherein said one or more polymorphisms in D3 comprises Ser9/Gly9, wherein said one or more polymorphisms in 5-HTT comprises LPR 480/520 bp, wherein said one or more polymorphisms in M1 comprises −12064-T/C, and wherein said one or more polymorphisms in 5-HT6 comprises 267-C/T.

7. The method of claim 4, wherein said one or more polymorphisms in ADRA1A consists of 4155-C/G, wherein said one or more polymorphisms in ADRA2A consists of −2211-A/T, and wherein said one or more polymorphisms in ANKK1 consists of −8882-C/G.

8. The method of claim 7, wherein said one or more polymorphisms in D3 consists of Ser9/Gly9, wherein said one or more polymorphisms in 5-HTT consists of LPR 480/520 bp, wherein said one or more polymorphisms in M1 consists of −12064-T/C, and wherein said one or more polymorphisms in 5-HT6 consists of 267-C/T.

9. The method of claim 2, further comprising determining the copy number of the wild type allele with respect to each polymorphism.

10. The method of claim 9, wherein the likelihood of a response to olanzapine treatment (LoR) in said patient can be predicted using the following algorithm: LoR=[1−(−0.173+1.552A1+1.361A2+2.273B1+1.893B2+0.007C1+0.298C2)], wherein A1=1A −4155-C/C genotype, A2=α1A −4155-C/G genotype, B1=α_{2A}−2211-A/A genotype, B2=α_{2A}−2211-A/T genotype, C1=ANKK1 −8882-C/C genotype, and C2=ANKK1 −8882-C/G genotype.

11. The method of claim 7, wherein the likelihood of a response to olanzapine treatment (LoR) in said patient can be predicted using the following algorithm: LoR=[1−(−3.443+1.745A1+1.909A2+2.574B1+1.901B2+0.681C1+1.033C2+1.691D1+0.801D2−18.217E1−18.204E2−18.589E3+0.732F1+1.099F2+22.508G1+23.778G2)] wherein A1=α_{1A}−4155-C/C genotype, A2=α1A −4155-C/G genotype, B1=α_{2A}−2211-A/A genotype, B2=α_{2A}−2211-A/T genotype, C1 ANKK1 −8882-C/C genotype, C2=ANKK1 −8882-C/G genotype, D1=D3 Ser9/Ser9 genotype, D2=D3 Ser9/Gly9 genotype, E1=5-HTT LPR 480 bp/480 bp genotype, E2=5-HTT LPR 480/520 bp genotype, E3=5-HTT LPR 520/520 bp genotype, F1=M1 −12064-T/T genotype and F2=M1 −12064-T/C genotype, G1=5-HT6267-C/C genotype and G2=5-HT6 267-C/T genotype.

12. The method of any on of claims 10 or 11, wherein said response is beneficial, as determined by an improvement of 20 points or more in the GAF scales, or at least a 30% decrease in PANSS values after olanzapine.

13. A method of determining the likelihood of a general response to olanzapine treatment in a patient comprising detecting the allelic forms of one or more polymorphisms in one or more genes in a sample of said patient, selected from the group consisting of: ADRA1A, ANKK1 and M1.

14. A method of determining the likelihood of a general response to olanzapine treatment in a patient comprising detecting the allelic forms of one or more polymorphisms in each of the following genes of said patient: ADRA1A, ANKK1 and M1.

15. The method of claim 13, further comprising detecting the allelic forms of one or more polymorphisms in one or more of the following genes of said patient: ChAT, 5HTT, and Neuregulin.

16. The method of any of claim 14, further comprising detecting the allelic forms of one or more polymorphisms in each of the following genes of said patient: ChAT, 5HTT, and Neuregulin.

17. The method of claim 13 or 14 or 15 or 16, wherein said one or more polymorphisms in ADRA1A comprises −4155 C/G, wherein said one or more polymorphisms in ANKK1 comprises −8882 C/G, and wherein said one or more polymorphisms in M1 comprises −12064 T/C.

18. The method of claim 17, wherein said one or more polymorphisms in ChAT comprises rs1880676 G/A, wherein said one or more polymorphisms in 5HTT comprises rs187294 2630 T/C, and wherein said one or more polymorphisms in Neuregulin comprises SNP8NRG221533 C/T.

19. The method of claim 16, wherein said one or more polymorphisms in ADRA1A consists of −4155 C/G, wherein said one or more polymorphisms in ANKK1 consists of −8882 C/G, and wherein said one or more polymorphisms in M1 consists of −12064 T/C.

20. The method of claim 19, wherein said one or more polymorphisms in ChAT consists of rs1880676 G/A, wherein said one or more polymorphisms in 5HTT consists of rs187294 2630 T/C, and wherein said one or more polymorphisms in Neuregulin consists of SNP8NRG221533 C/T.

21. The method of claim 19, wherein said likelihood of an overall response to olanzapine treatment in said patient (LoR) is calculated according to the following algorithm: [1−(−2.59+0.915A1+0.526A2+1.686B1+1.888B2+0.743C1+1.889C2)] as measured by PANSS, wherein A1=Alpha-1A adrenergic receptor rs2644627−4155C/C, A2=Alpha-1A adrenergic receptor rs2644627−4155C/G, B1=ANKK1 rs3897584−8882C/C, B2=ANKK1 rs3897584−8882 C/G, C1=Muscarinic receptor M1 rs12295208−12064T/T, and C2=Muscarinic receptor M1 rs12295208−12064 T/C.

22. The method of claim 20, wherein said likelihood of an overall response to olanzapine treatment in said patient (LoR) is calculated according to the following algorithm: =[1−(−5.402+1.602A1+1.108A2+1.212B1+1.965B2+1.399C1+2.814C2+1.118D1+0.068D2+1.708E1+1.952E2+1.097F1−0.806F2)], as measured by PANSS, wherein A1=Alpha-1A adrenergic receptor rs2644627−4155 C/C, A2=Alpha-1A adrenergic receptor rs2644627−4155 C/G, B1=ANKK1 rs3897584−8882 C/C, B2=ANKK1 rs3897584−8882 C/G, C1=Muscarinic receptor M1 rs12295208−12064 T/T, C2=Muscarinic receptor M1 rs122952098−12064 T/C, D1=Choline Acetyltransferase ChAT rs1880676 G/G, D2=Choline Acetyltransferase ChAT rs1880676 G/A, E1=5-HTT rs187294 2630 T/T, E2=5-HTT rs187294 2630 T/C, F1=Neuregulin 1 SNP8NRG221533 C/C and F2=Neuregulin 1 SNP8NRG221533 C/T.

23. The method of claim 21 or 22, wherein said response is measured by PANSS, and said response is a therapeutically effective response comprises at least a 30% decrease in PANSS.

24. The method of claim 19, wherein said likelihood of a overall response to olanzapine treatment in said patient (LoR) is calculated according to the following algorithm: likelihood of response (LoR)=[1−(−1.897+1.068A1+0.799A2+1.023B1+1.258B2+0.601C1+1.285C2)] as measured by GAF, wherein A1=Alpha-1A adrenergic receptor rs2644627-4155C/C, A2=Alpha-1A adrenergic receptor rs2644627−4155C/G, B1=ANKK1 rs3897584−8882C/C, B2=ANKK1 rs3897584−8882 C/G, C1=Muscarinic receptor M1 rs12295208−12064T/T, and C2=Muscarinic receptor M1 rs12295208−12064 T/C

25. The method of claim 20, wherein said likelihood of an overall response to olanzapine treatment in said patient (LoR) is calculated according to the following algorithm: =[1−(5.916+1.646A1+1.387A2+0.925B1+1.318B2+0.648C1+1.480C2+0.973D1+0.418D2+2.671 A1+2.888E2+2.471 A1+0.145F2)], wherein A1=Alpha-1A adrenergic receptor rs2644627−4155 C/C, A2=Alpha-1A adrenergic receptor rs2644627−4155 C/G, B1=ANKK1 rs3897584−8882 C/C, B2=ANKK1 rs3897584−8882 C/G, C1=Muscarinic receptor M1 rs12295208−12064 T/T, C2=Muscarinic receptor M1 rs122952098−12064 T/C, D1=Choline Acetyltransferase ChAT rs1880676 G/G, D2=Choline Acetyltransferase ChAT rs1880676 G/A, E1=5-HTT rs187294 2630 T/T, E2=5-HTT rs187294 2630 T/C, F1=Neuregulin 1 SNP8NRG221533 C/C and F2=Neuregulin 1 SNP8NRG221533 C/T.

26. The method of claim 24 or 25, wherein said response is measured by GAF, and said response is a therapeutically effective response comprises an improvement of 20 points or more in GAF scales.

27. A method of determining the likelihood of improvement in positive symptoms to olanzapine treatment in a patient comprising detecting the allelic forms of one or more polymorphisms in one or more genes of said patient selected from the group consisting of: ANKK1, M1, and ADRA1A.

28. A method of determining the likelihood of improvement in positive symptoms to olanzapine treatment in a patient comprising detecting the allelic forms of one or more polymorphisms in each of the following genes of said patient: ANKK1, M1, and ADRA1A.

29. The method of claim 27 or 28, wherein said one or more polymorphisms in ANKK1 comprises −8882 C/G, wherein said one or more polymorphisms in M1 comprises −12064 T/C, and wherein said one or more polymorphisms in ADRA1A comprises −4155C/G.

30. The method of claim 28, wherein said one or more polymorphisms in ANKK1 consists of −8882 C/G, wherein said one or more polymorphisms in M1 consists of −12064 T/C, and wherein said one or more polymorphisms in ADRA1A consists of −4155C/G.

31. The method of claim 30, wherein said likelihood of improvement in positive symptoms to olanzapine treatment in said patient (LoR) is calculated according to the following algorithm: LoR=[1−(−2.08+1.748A1+1.851A2+0.429B1+1.338B2−0.792C1−0.217C2)] wherein A1=ANKK1 rs3897584−8882 C/C, A2=ANKK1 rs3897584−8882 C/G, B1=Muscarinic receptor M1 rs12295208−12064 T/T, B2=Muscarinic receptor M1 rs12295208−12064 T/C, C1=Alpha-1A adrenergic receptor rs2644627−4155 C/C, and C2=Alpha-1A adrenergic receptor rs2644627−4155 C/G.

32. The method of claim 31, wherein said improvement in positive symptoms to olanzapine treatment in a patient response is measured by PANSS, and said improvement in positive symptoms comprises at least a 30% decrease in positive PANSS scores.

33. A method of determining the likelihood of improvement in negative symptoms to olanzapine treatment in a patient comprising detecting the allelic forms of one or more polymorphisms in one or more genes of said patient selected from the group consisting of: RXFP3, RFXP4, and ChAT.

34. A method of determining the likelihood of improvement in negative symptoms to olanzapine treatment in a patient comprising detecting the allelic forms of one or more polymorphisms in each of the following genes of said patient: RXFP3, RXFP4, and ChAT.

35. The method of claim 33 or 34, wherein said one or more polymorphisms in RXFP3 comprises −903 A/C, wherein said one or more polymorphisms in RXFP4 comprises −3768 A/T, and wherein said one or more polymorphisms ChAT comprises rs8178984 C/C.

36. The method of claim 34, wherein said one or more polymorphisms in RXFP3 consists of −903 A/C, wherein said one or more polymorphisms in RXFP4 consists of −3768 A/T, and wherein said one or more polymorphisms CHAT consists of rs8178984 C/C.

37. The method of claim 36, wherein said likelihood of improvement in negative symptoms to olanzapine treatment in said patient (LoR) is calculated according to the following algorithm: LoR=[1−(−0.221−0.24A1+0.764A2+0.308B1−0.482B21.812C1)], wherein A1=RXFP3 rs7702361−903 A/A, A2=RXFP3 rs7702361−903 C/C, B1=RXFP4 rs11264422−3678 A/A, B2=RXFP4 rs11264422−3678 A/T and C1=Choline Acetyltransferase ChAT rs8178984 C/C.

38. The method of claim 37, wherein said improvement in negative symptoms is measured by PANSS, and said improvement comprises at least a 30% decrease in negative PANSS score.

39. A method of determining the likelihood of an improvement in general psychopathology in response to olanzapine treatment in a patient comprising detecting the allelic forms of one or more polymorphisms in one or more genes of said patient selected from the group consisting of: ChAT, M1 and ANKK1.

40. A method of determining the likelihood of an improvement in general psychopathology in response to olanzapine treatment in a patient comprising detecting the allelic forms of one or more polymorphisms in each of the following genes of said patient: ChAT, M1 and ANKK1.

41. The method of claim 39 or 40, wherein said one or more polymorphisms in ChAT comprises rs1880676 G/A and rs8178984 C/C, wherein said one or more polymorphisms in M1 comprises −12064 T/T or T/C, and wherein said one or more polymorphisms in ANKK1 comprises −8882 C/G.

42. The method of claim 40, wherein said one or more polymorphisms in ChAT consists of rs1880676 G/A and rs8178984 C/C, wherein said one or more polymorphisms in M1 consists of −12064 T/T or T/C, and wherein said one or more polymorphisms in ANKK1 consists of −8882 C/G.

43. The method of claim 42, wherein said likelihood of improvement in general psychopathology in response to olanzapine treatment in said patient (LoR) is calculated according to the following algorithm: LoR=[1−(−3.461+1.181A1+1.69B1+0.658B2+0.902C1+2.189C2+0.657D1+1.246D2)], wherein A1=Choline Acetyltransferase ChAT rs8178984 C/C, B1=Choline Acetyltransferase ChAT rs1880676 G/G, B2=Choline Acetyltransferase ChAT rs1880676 G/A, C1=Muscarinic receptor M1 rs12295208−12064 T/T, C2=Muscarinic receptor M1 rs12295208−12064 T/C, D1=ANKK1 rs3897584−8882 C/C and D2=ANKK1 rs3897584−8882 C/G.

44. The method of claim 43, wherein said improvement is measured by PANSS, and comprises at least a 30% decrease in general psychopathology PANSS score.

45. A kit for determining a genotype of an individual, wherein said kit comprises oligonucleotides for detection of alleles of each polymorphism in the group consisting of: α1A−4155-C/C genotype, α1A −4155-C/G genotype, α_{2A}−2211-A/A genotype, α_{2A}−2211-A/T genotype, ANKK1 −8882-C/C genotype, and ANKK −8882-C/G.

46. The kit of claim 45, wherein said oligonucleotides comprise oligonucleotides with sequences selected from the group consisting of: SEQ ID NO:1, SEQ ID NO:2, SEQ ID NO:7, SEQ ID NO:8, SEQ ID NO:9, and SEQ ID NO:10.

47. A kit for determining a genotype of an individual, wherein said kit comprises oligonucleotides for detection of alleles of each polymorphism in the group consisting of: α1A −4155-C/C genotype, α1A −4155-C/G genotype, α_{2A}−2211-A/A genotype, α_{2A}−2211-A/T genotype, ANKK1 −8882-C/C genotype, ANKK1 −8882-C/G genotype, D3 Ser9/Ser9 genotype, D3 Ser9/Gly9 genotype, 5-HTT LPR 480 bp/480 bp genotype, 5-HTT LPR 480/520 bp genotype, 5-HTT LPR 520/520 bp genotype, M1 −12064-T/T genotype and M1 −12064-T/C genotype, 5-HT6 267-C/C genotype and 5-HT6 267-C/T genotype.

48. The kit of claim 47, wherein said oligonucleotides comprise oligonucleotides with sequences selected from the group consisting of: SEQ ID NO:1, SEQ ID NO:2, SEQ ID NO:7, SEQ ID NO:8, SEQ ID NO:9, and SEQ ID NO:10, SEQ ID NO:22, SEQ ID NO:23, SEQ ID NO:40, SEQ ID NO:41, SEQ ID NO:42, SEQ ID NO:43, SEQ ID NO:36 and SEQ ID NO:37.

49. A kit for determining a genotype of an individual, wherein said kit comprises oligonucleotides for detection of alleles of each polymorphism in the group consisting of: Alpha-1A adrenergic receptor rs2644627−4155C/C, Alpha-1A adrenergic receptor rs2644627−4155C/G, ANKK1 rs3897584−8882C/C, ANKK1 rs3897584−8882 C/G, Muscarinic receptor M1 rs12295208−12064T/T, and Muscarinic receptor M1 rs12295208−12064 T/C.

50. The kit of claim 49, wherein said oligonucleotides comprise oligonucleotides with sequences selected from the group consisting of: SEQ ID NO:1, SEQ ID NO:2, SEQ ID NO:9, SEQ ID NO:10, SEQ ID NO:42, SEQ ID NO:43.

51. A kit for determining a genotype of an individual, wherein said kit comprises oligonucleotides for detection of alleles of each polymorphism in the group consisting of: Alpha-1A adrenergic receptor rs2644627−4155 C/C, Alpha-1A adrenergic receptor rs2644627−4155 C/G, ANKK1 rs3897584−8882 C/C, ANKK1 rs3897584−8882 C/G, Muscarinic receptor M1 rs12295208−12064 T/T, Muscarinic receptor M1 rs122952098−12064 T/C, Choline Acetyltransferase ChAT rs1880676 G/G, Choline Acetyltransferase ChAT rs11880676 G/A, 5-HTT rs1187294 2630 T/T, 5-HTT rs11872924 2630 T/C, Neuregulin 1 SNP8NRG221533 C/C and Neuregulin 1 SNP8NRG221533 C/T.

52. The kit of claim 51, wherein said oligonucleotides comprise oligonucleotides with sequences selected from the group consisting of: SEQ ID NO: 1, SEQ ID NO:2, SEQ ID NO:9, SEQ ID NO: 10, SEQ ID NO:42, SEQ ID NO:11, SEQ ID NO:12, SEQ ID NO:13, SEQ ID NO:38, SEQ ID NO:43, SEQ ID NO:44, SEQ ID NO:45, SEQ ID NO:46, SEQ ID NO:47 and SEQ ID NO:39.

53. A kit for determining a genotype of an individual, wherein said kit comprises oligonucleotides for detection of alleles of each polymorphism in the group consisting of: RXFP3 rs7702361−903 A/A, RXFP3 rs7702361−903 C/C, RXFP4 rs11264422−3678 A/A, RXFP4 rs11264422−3678 A/T and Choline Acetyltransferase ChAT rs8178984 C/C genotypes.

54. The kit of claim 53, wherein said oligonucleotides comprise oligonucleotides with sequences selected from the group consisting of: SEQ ID NO:48, SEQ ID NO:49, SEQ ID NO:50, SEQ ID NO:51, SEQ ID NO:14 and SEQ ID NO:15.

55. A kit for determining a genotype of an individual, wherein said kit comprises oligonucleotides for detection of alleles of each polymorphism in the group consisting of: Choline Acetyltransferase ChAT rs8178984 C/C, Choline Acetyltransferase ChAT rs1880676 G/G, Choline Acetyltransferase ChAT rs1880676 G/A, Muscarinic receptor M1 rs12295208−12064 T/T, Muscarinic receptor M1 rs12295208−12064 T/C, ANKK1 rs3897584−8882 C/C and ANKK1 rs3897584−8882 C/G.

56. The kit of claim 55, wherein said oligonucleotides comprise oligonucleotides with sequences selected from the group consisting of: SEQ ID NO:9, SEQ ID NO:10, SEQ ID NO:11, SEQ ID NO:12, SEQ ID NO: 13, SEQ ID NO:42, SEQ ID NO:43, SEQ ID NO:14 and SEQ ID NO:15.

57. An isolated nucleic acid comprising a polymorphism selected from the group consisting of: ADRA1A −4155 C/G, ADRA2A −2211 A/T, ANKK1 −8882 G/C, CHAT rs1880676, CHAT rs8178984, DRD3 Ser9Gly, 5-HT6 267 C/T, 5-HTT (SLC6A4) 2630 C/T, 5-HTT (SLC6A4) LPR 480/520 bp, M1 −12,064 T/C, Neuregulin 1221533, RXFP3 rs7702361 A/C, and RXFP4 rs11264422 A/T.


SEQUENCE LISTING (55 sequences)

SEQ ID NO: 1 - DNA, 20 bp, Artificial Sequence
  Description of Artificial Sequence Synthetic oligonucleotide
gcctgtgaccatcaatcctt

SEQ ID NO: 2 - DNA, 20 bp, Artificial Sequence
  Description of Artificial Sequence Synthetic oligonucleotide
gtcgcataggagggtcagaa

SEQ ID NO: 3 - DNA, 52 bp, Artificial Sequence
  Description of Artificial Sequence Synthetic oligonucleotide
catctttatgaccccagagcatactcstctccactccacctacccatgtgta

SEQ ID NO: 4 - DNA, 52 bp, Artificial Sequence
  Description of Artificial Sequence Synthetic oligonucleotide
aaatattctactccctcttccccttawtgaaggatgctgtgtgtacatctga

SEQ ID NO: 5 - DNA, 52 bp, Artificial Sequence
  Description of Artificial Sequence Synthetic oligonucleotide
ctccctcctacatggccaagtactgasgatagagcagagaacaagattcctg

SEQ ID NO: 6 - DNA, 51 bp, Artificial Sequence
  Description of Artificial Sequence Synthetic oligonucleotide
tgccccacaggtgtagttcaggtggcyactcagctggctcagagatgccat

SEQ ID NO: 7 - DNA, 20 bp, Artificial Sequence
  Description of Artificial Sequence Synthetic oligonucleotide
tagcagggtgcaagctagtg

SEQ ID NO: 8 - DNA, 20 bp, Artificial Sequence
  Description of Artificial Sequence Synthetic oligonucleotide
gtcacatcaggaagggctca

SEQ ID NO: 9 - DNA, 20 bp, Artificial Sequence
  Description of Artificial Sequence Synthetic oligonucleotide
aaccttcaccccgggagatt

SEQ ID NO: 10 - DNA, 20 bp, Artificial Sequence
  Description of Artificial Sequence Synthetic oligonucleotide
ctgcacatgcacccactgaa

SEQ ID NO: 11 - DNA, 21 bp, Artificial Sequence
  Description of Artificial Sequence Synthetic oligonucleotide
agatgtggccggaatgcagag

SEQ ID NO: 12 - DNA, 22 bp, Artificial Sequence
  Description of Artificial Sequence Synthetic oligonucleotide
gagatgtggccggaatgcagaa

SEQ ID NO: 13 - DNA, 26 bp, Artificial Sequence
  Description of Artificial Sequence Synthetic oligonucleotide
catacctactgtgctcagtgcttcat

SEQ ID NO: 14 - DNA, 20 bp, Artificial Sequence
  Description of Artificial Sequence Synthetic oligonucleotide
agcgagcagagacttcctca

SEQ ID NO: 15 - DNA, 20 bp, Artificial Sequence
  Description of Artificial Sequence Synthetic oligonucleotide
tagaagggggcacagaacac

SEQ ID NO: 16 - DNA, 51 bp, Artificial Sequence
  Description of Artificial Sequence Synthetic oligonucleotide
ctggggggccgtttgccctagagatgygggtcctgcaccgcctctgtttgg

SEQ ID NO: 17 - DNA, 50 bp, Artificial Sequence
  Description of Artificial Sequence Synthetic oligonucleotide
ccgccggccatgctgaacgcgctgtaygggcgctgggtgctggcgcgcgg

SEQ ID NO: 18 - DNA, 20 bp, Artificial Sequence
  Description of Artificial Sequence Synthetic oligonucleotide
ggcgttgccgctctgaatgc

SEQ ID NO: 19 - DNA, 23 bp, Artificial Sequence
  Description of Artificial Sequence Synthetic oligonucleotide
gagggactgagctggacaaccac

SEQ ID NO: 20 - DNA, 2726 bp, Homo sapiens
acagcccgggaccagcctccccgcgcagcctggcaggtctcctggaggcaaggcgaccttgcttgccctctcttgcagaataacaaggggcttagccacaggagttgctggcaagtggaaagaagaacaaatgagtcaatcccgacgtgtcaatcccgacgatagagagctcggaggtgatccacaaatccaagcacccagagatcaattgggatccttggcagatggacatcagtgtcatttactaaccagcaggatggagacgacgcccttgaattctcagaagcagctatcagcgtgtgaagatggagaagattgtcaggaaaacggagttctacagaaggttgttcccaccccaggggacaaagtggagtccgggcaaatatccaatgggtactcagcagttccaagtcctggtgcgggagatgacacacggcactctatcccagcgaccaccaccaccctagtggctgagcttcatcaaggggaacgggagacctggggcaagaaggtggatttccttctctcagtgattggctatgctgtggacctgggcaatgtctggcgcttcccctacatatgttaccagaatggagggggggcattcctcctcccctacaccatcatggccatttttgggggaatcccgctcttttacatggagctcgcactgggacagtaccaccgaaatggatgcatttcaatatggaggaaaatctgcccgattttcaaagggattggttatgccatctgcatcattgccttttacattgcttcctactacaacaccatcatggcctgggcgctatactacctcatctcctccttcacggaccagctgccctggaccagctgcaagaactcctggaacactggcaactgcaccaattacttctccgaggacaacatcacctggaccctccattccacgtcccctgctgaagaattttacacgcgccacgtcctgcagatccaccggtctaaggggctccaggacctggggggcatcagctggcagctggccctctgcatcatgctgatcttcactgttatctacttcagcatctggaaaggcgtcaagacctctggcaaggtggtgtgggtgacagccaccttcccttatatcatcctttctgtcctgctggtgaggggtgccaccctccctggagcctggaggggtgttctcttctacttgaaacccaattggcagaaactcctggagacaggggtgtggatagatgcagccgctcagatcttcttctctcttggtccgggctttggggtcctgctggcttttgctagctacaacaagttcaacaacaactgctaccaagatgccctggtgaccagcgtggtgaactgcatgacgagcttcgtttcgggatttgtcatcttcacagtgctcggttacatggctgagatgaggaatgaagatgtgtctgaggtggccaaagacgcaggtcccagcctcctcttcatcacgtatgcagaagcgatagccaacatgccagcgtccactttctttgccatcatcttctttctgatgttaatcacgctgggcttggacagcacgtttgcaggcttggagggggtgatcacggctgtgctggatgagttcccacacgtctgggccaagcgccgggagcggttcgtgctcgccgtggtcatcacctgcttctttggatccctggtcaccctgacttttggaggggcctacgtggtgaagctgctggaggagtatgccacggggcccgcagtgctcactgtcgcgctgatcgaagcagtcgctgtgtcttggttctatggcatcactcagttctgcagggacgtgaaggaaatgctcggcttcagcccggggtggttctggaggatctgctgggtggccatcagccctctgtttctcctgttcatcatttgcagttttctgatgagcccgccacaactacgacttttccaatataattatccttactggagtatcatcttgggttactgcataggaacctcatctttcatttgcatccccacatatatagcttatcggttgatcatcactccagggacatttaaagagcgtattattaaaagtattaccccagaaacaccaacagaaattccttgtggggacatccgcttgaatgctgtgtaacacactcaccgagaggaaaaaggcttctccacaacctcctcctccagttctgatgaggcacgcctgccttctcccctccaagtgaatgagtttccagctaagcctgatgatggaagggccttctccacagggacacagtctggtgcccagactcaaggcctccagccacttatttccatggattcccctggacatattcccatggtagactgtgacacagctgagctggcctattttggacgtgtgaggatgtggatggaggtgatgaaaaccaccctatcatcagttaggattaggtttagaatcaagtctgtgaaagtctcctgtatcatttcttggtatgatcattggtatctgatatctgtttgcttctaaaggtttcactgttcatgaatacgtaaactgcgtaggagagaacagggatgctatctcgctagccatatattttctgagtagcatatataattttattgctggaatctactagaaccttctaatccatgtgctgctgtggcatcaggaaaggaagatgtaagaagctaaaatgaaaaatagtgtgtccatgcaaaaaaaaaaa

SEQ ID NO: 21 - DNA, 2304 bp, Homo sapiens
gaattccgaatcatgtgcagaatgctgaatcttcccccagccaggacgaataagacagcgcggaaaagcagattctcgtaattctggaattgcatgttgcaaggagtctcctggatcttcgcacccagcttcgggtagggagggagtccgggtcccgggctaggccagcccggcaggtggagagggtccccggcagccccgcgcgcccctggccatgtctttaatgccctgccccttcatgtggccttctgagggttcccagggctggccagggttgtttcccacccgcgcgcgcgctctcacccccagccaaacccacctggcagggctccctccagccgagaccttttgattcccggctcccgcgctcccgcctccgcgccagcccgggaggtggccctggacagccggacctcgcccggccccggctgggaccatggtgtttctctcgggaaatgcttccgacagctccaactgcacccaaccgccggcaccggtgaacatttccaaggccattctgctcggggtgatcttggggggcctcattcttttcggggtgctgggtaacatcctagtgatcctctccgtagcctgtcaccgacacctgcactcagtcacgcactactacatcgtcaacctggcggtggccgacctcctgctcacctccacggtgctgcccttctccgccatcttcgaggtcctaggctactgggccttcggcagggtcttctgcaacatctgggcggcagtggatgtgctgtgctgcaccgcgtccatcatgggcctctgcatcatctccatcgaccgctacatcggcgtgagctacccgctgcgctacccaaccatcgtcacccagaggaggggtctcatggctctgctctgcgtctgggcactctccctggtcatatccattggacccctgttcggctggaggcagccggcccccgaggacgagaccatctgccagatcaacgaggagccgggctacgtgctcttctcagcgctgggctccttctacctgcctctggccatcatcctggtcatgtactgccgcgtctacgtggtggccaagagggagagccggggcctcaagtctggcctcaagaccgacaagtcggactcggagcaagtgacgctccgcatccatcggaaaaacgccccggcaggaggcagcgggatggccagcgccaagaccaagacgcacttctcagtgaggctcctcaagttctcccgggagaagaaagcggccaaaacgctgggcatcgtggtcggctgcttcgtcctctgctggctgccttttttcttagtcatgcccattgggtctttcttccctgatttcaagccctctgaaacagtttttaaaatagtattttggctcggatatctaaacagctgcatcaaccccatcatatacccatgctccagccaagagttcaaaaaggcctttcagaatgtcttgagaatccagtgtctctgcagaaagcagtcttccaaacatgccctgggctacaccctgcacccgcccagccaggccgtggaagggcaacacaaggacatggtgcgcatccccgtgggatcaagagagaccttctacaggatctccaagacggatggcgtttgtgaatggaaatttttctcttccatgccccgtggatctgccaggattacagtgtccaaagaccaatcctcctgtaccacagcccggacgaagtctcgctctgtcaccaggctggagtgcagtggcatgatcttggctcactgcaacctccgcctcccgggttcaagagattctcctgcctcagcctcccaagcagctgggactacagggatgtgccaccaggccgacgccaccaggcccagctaatttttgtatttttagtagagacggggtttcaccatgttggccaggatgatctcgatctcttgacctcatgatctgcctgcctcagcctcccaaagtgctgggattacaggcgtgagccaccgtgcccggcccaactattttttttttttatcttttttaacagtgcaatcctttctgtggatgaaatcttgctcagaagctcaatatgcaaaagaaagaaaaacagcagggctggacggatgttgggagtggggtaagaccccaaccactcagaaccacccccccaacacacacacacattctctccatggtgactggtgaggggcctctagagggtacatagtacaccatggagcacggtttaagcaccactggactacacattcttctgtggcagttatcttaccttcccatagacacccagcccatagccattggtt

SEQ ID NO: 22 - DNA, 21 bp, Artificial Sequence
  Description of Artificial Sequence Synthetic oligonucleotide
gctctatctccaactctcaca

SEQ ID NO: 23 - DNA, 21 bp, Artificial Sequence
  Description of Artificial Sequence Synthetic oligonucleotide
aagtctactcacctccaggta

SEQ ID NO: 24 - DNA, 51 bp, Artificial Sequence
  Description of Artificial Sequence Synthetic oligonucleotide
caccagagatgtggccggaatgcagaratgaagcactgagcacagtaggta

SEQ ID NO: 25 - DNA, 52 bp, Artificial Sequence
  Description of Artificial Sequence Synthetic oligonucleotide
ccaaatgtagccacacatcatagtcayctagattcctgggtctaccccagac

SEQ ID NO: 26 - DNA, 3653 bp, Homo sapiens
gcgctcggcgcccaccaggcggacgcccaggagaacccctgcctccgtcgcggctcctggagagctgatcgttcacctgccccggcccgcctgaggacgggggtgccttcatgcggcccccacactcctcaccccgccgccgccgccgtccccgagctccgcacagtgcgccccagccccagcagggcgcacaactttggaagtctcgcggcgctccgagaggcggcagagtccgcgccccagccccgggccgggccgggccagaaccgcagcgtctgggggaagccagagagtcggtaatcgcttcggggatgtaaggcgacagacataggacccccgagctcgcatcagcacccttcggctgcctcccggggtgggggcgggccccgcacacggtaagacctcttgctttcgctcaggctcaagattcaagatacagatattgatatgtatatatatatttaatttcctgtcatccttccaagttatcaggccaccgatgatttttgttctcccttcttgaagaataaatctctctttacccatcggctctccctactctctcccgccgcttagaaataaaacttggctgtattaggagctcggagcaagaaggcgcccaccgagagcgtctgaagcgcgagccaggcgcagttcgcgggacccgggccatgggccgctagcggtcctccagttcgggcccggcctccctgcggccccctccctatgtgagccgcagccaggcgagcggggcgccggaggaagaggaggacccacgggcgccgggccggaaggcagctggcagcaggcccaggccagcgggcgcccgcgttcatgttccgccaggagcagccgttggccgagggcagctttgcgcccatgggctccctgcagccggacgcgggcaacgcgagctggaacgggaccgaggcgccggggggcggcgcccgggccaccccttactccctgcaggtgacgctgacgctggtgtgcctggccggcctgctcatgctgctcaccgtgttcggcaacgtgctcgtcatcatcgccgtgttcacgagccgcgcgctcaaggcgccccaaaacctcttcctggtgtctctggcctcggccgacatcctggtggccacgctcgtcatccctttctcgctggccaacgaggtcatgggctactggtacttcggcaaggcttggtgcgagatctacctggcgctcgacgtgctcttctgcacgtcgtccatcgtgcacctgtgcgccatcagcctggaccgctactggtccatcacacaggccatcgagtacaacctgaagcgcacgccgcgccgcatcaaggccatcatcatcaccgtgtgggtcatctcggccgtcatctccttcccgccgctcatctccatcgagaagaagggcggcggcggcggcccgcagccggccgagccgcgctgcgagatcaacgaccagaagtggtacgtcatctcgtcgtgcatcggctccttcttcgctccctgcctcatcatgatcctggtctacgtgcgcatctaccagatcgccaagcgtcgcacccgcgtgccacccagccgccggggtccggacgccgtcgccgcgccgccggggggcaccgagcgcaggcccaacggtctgggccccgagcgcagcgcgggcccggggggcgcagaggccgaaccgctgcccacccagctcaacggcgcccctggcgagcccgcgccggccgggccgcgcgacaccgacgcgctggacctggaggagagctcgtcttccgaccacgccgagcggcctccagggccccgcagacccgagcgcggtccccggggcaaaggcaaggcccgagcgagccaggtgaagccgggcgacagcctgccgcggcgcgggccgggggcgacggggatcgggacgccggctgcagggccgggggaggagcgcgtcggggctgccaaggcgtcgcgctggcgcgggcggcagaaccgcgagaagcgcttcacgttcgtgctggccgtggtcatcggagtgttcgtggtgtgctggttccccttcttcttcacctacacgctcacggccgtcgggtgctccgtgccacgcacgctcttcaaattcttcttctggttcggctactgcaacagctcgttgaacccggtcatctacaccatcttcaaccacgatttccgccgcgccttcaagaagatcctctgtcggggggacaggaagcggatcgtgtgaggtttccgctggcgcccgcgtagactcacgctgactgcaggcagcggggggcatcgaggggtgcttagccccagggcactcagaaacccgggcgctgcctgctctgcgtttcctcgtctggggtggctctgcagcctcctgcgggcgggcgtctgctgctcctacaagggaagcttcttgctgccaggcccacacatccccagttgttggtttggccactcttgacctggagccatcttcctagtgggccacccctaatcactattgcttcctaaaggtattttcaccctcttcgcctggtacagccctcacagctcttcagagcaagcactggactacaagggcatggctcacaaaaggttaatggatgggggttacctagccctggctaattccccttccattcccaactctctctctctttttgaagaaaaatgctaagggcagccctgcctgccctccccatcccccgctgtaaatatacactatttttgatagcacacatggggcccccatatctcttggccttggttttgatgttgaaatcctggccttgggagagatgccttccaggcagacacagctgtctggttcaggccaagcccctttgcaatgcaagccctttctggtgttatgaagtccctctatgtcgtcgttttcaccagcaactggtgactgtcccttcgacacggacctgctttgagatttcctgacagggaaaagatttctgtccatttttttcctgtgcctaacagcataattgccttttcctatgtaaatattatgatggtggatcaagacataagtaaatgagcctttctgcctcacatcagccctgtgtataaagccattattctctgatgcactgtttgccccagtaactcactttaaaacctctctttccagtgttccctctctccctccagggccactgcttgaagaagaatatgtatgtttctatcttgtatgtctgtgtgcccctcctgccccgaaagtgctgactatggggaaatcttttagctgctgtttttagactccaaggagtggaaattatgtggaagaagcaaacctgatacaatttgcccaaggtaaacagtttgaaaagacaaatgggcctgccaaactgtacagtttcttccccaagagctgttaggtatcaaaatgttgtcctttcccccctccgtgcttttctggttgagatcatgtcattgatgaactgccaaagtcaggggaggagggcagagactttgtgtttacatctgcatttctacatgttttagacagagacaatttaaggcctgcactcttatttcactaaagaaaaactaatgtcagcacatgttgctaatgacagtggatttttttttaaataaaaaagtttacagatcaaatgtgaaataaatatgaatggagtggtcaaa

SEQ ID NO: 27 - DNA, 52 bp, Artificial Sequence
  Description of Artificial Sequence Synthetic oligonucleotide
actaaaaaagagatatatgatatttggyaaaataaagatacatggcttccag

SEQ ID NO: 28 - DNA, 2863 bp, Homo sapiens
tggggctcaaattgggtgccctggtgaaggaggggggcacactccagaacctagtccaaccccagacgctgcctgaggcttccctccagctcccctcccttccttttctccctttcctccctccctctctttccctttctccctccccgctaaggctggcgtgccagggggtgggacatgccaatcactggctgtgcctctcccgctgccagcacagggcgcagctccccctgggagccaggtgtttgggtccctggagacgccgcaggcccccagggaggcagtggggctgaggaccctacagacccctcttcagccccgtggtgatgactttcccctgaggaagccctgtagcgtgcctggaggaaggggctctccaaccccagccccacctagccaccatgaacacttcagccccacctgctgtcagccccaacatcaccgtcctggcaccaggaaagggtccctggcaagtggccttcattgggatcaccacgggcctcctgtcgctagccacagtgacaggcaacctgctggtactcatctctttcaaggtcaacacggagctcaagacagtcaataactacttcctgctgagcctggcctgtgctgacctcatcatcggtaccttctccatgaacctctataccacgtacctgctcatgggccactgggctctgggcacgctggcttgtgacctctggctggccctggactatgtggccagcaatgcctccgtcatgaatctgctgctcatcagctttgaccgctacttctccgtgactcggcccctgagctaccgtgccaagcgcacaccccgccgggcagctctgatgatcggcctggcctggctggtttcctttgtgctctgggccccagccatcctcttctggcagtacctggtaggggagcggacagtgctagctgggcagtgctacatccagttcctctcccagcccatcatcacctttggcacagccatggctgccttctacctccctgtcacagtcatgtgcacgctctactggcgcatctaccgggagacagagaaccgagcacgggagctggcagcccttcagggctccgagacgccaggcaaagggggtggcagcagcagcagctcagagaggtctcagccaggggctgagggctcaccagagactcctccaggccgctgctgtcgctgctgccgggcccccaggctgctgcaggcctacagctggaaggaagaagaggaagaggacgaaggctccatggagtccctcacatcctcagagggagaggagcctggctccgaagtggtgatcaagatgccaatggtggaccccgaggcacaggcccccaccaagcagcccccacggagctccccaaatacagtcaagaggccgactaagaaagggcgtgatcgagctggcaagggccagaagccccgtggaaaggagcagctggccaagcggaagaccttctcgctggtcaaggagaagaaggcggctcggaccctgagtgccatcctcctggccttcatcctcacctggacaccgtacaacatcatggtgctggtgtccaccttctgcaaggactgtgttcccgagaccctgtgggagctgggctactggctgtgctacgtcaacagcaccatcaaccccatgtgctacgcactctgcaacaaagccttccgggacacctttcgcctgctgctgctttgccgctgggacaagagacgctggcgcaagatccccaagcgccctggctccgtgcaccgcactccctcccgccaatgctgatagtcccctctcctgcatccctccaccccagtccccgggaaaggccggtgggaagagggcaggggctgcatcctcagccccagggccctgctcaggcctcacctggcttcccaggaccctgggtcaccttcctgggcagcccagagagaccctgccaactttccagacttcgctattcccaggcagggagggaaacccggggaactggtttttctgttccctgctgggtgggaatgcgctcttcaccaggaagaaggcccgggaggaggatccgggctttggactccttgtttgcctttaggcaggaagtcaggagccagcagggcgggccaggagaaagaaggcttaacattaagtattccttggcccagcagcggcccagattgcggtgtgagatggtgccccctggggggcacagccagaaactgaactggccgctgggagaaaagccagatgacagggagctggggaatcccctcgcttcataggcagagcccgcccacctgggccctaggcatactctccaggattgtccacaaatgtcctcagagggtccctaggtgggtcaactccaaggcaaatgtccaagcatcagcaagacaatgacactggaagggtccggcttggctagtcacatatcaagtcccgaggcagcaacaggaccaggagccaggtgtcctgactgtcctacaatatcattttcctgggagtgggagtcaagtgtgcctgctatccagccgcaaatccataccccctgccccagagaagcctcagtccctccctcctggctcacagccaccacctggatggatctgctccatgcagatctagccaggcctcccgcatgctgcctgcctccggccctgccccacacaggcctggcccagccagcaggttctctcctgtgagctccccaatccaacccatgcatggcctcccagccacccggatctccaggcccagcctggccccaaatgttctttcctttcatcctcagcaagtgctgagtctgtgaataaagccacataaccagcgggcaaaaaaaaaaaaaaaaaaaaaaaaaaaaaaaaaaaaaaaaa

SEQ ID NO: 29 - DNA, 2485 bp, Homo sapiens
tggggttggggaagtgcggtgactgggaaatgctgagctaggggcaggaggcatgggcgggacagtgttctgtgcccccttctagagcctaaatttgttgcccgagttcctccgggaagcgctccgggtagattctgggggccgggagctgagatccctgggcggggagctggggaagggatggggctgaggacagcgaagaagagggggcttgggggaggggggaaatggaagagagaggagggaggaggtacaagaggaaggagagaagtgcggccagcttgctttctccagtcgggtggccgcggggacccgggcgacgtcggaggccctgccgggaacccaggctgcagcccccacccccgcgctgcgacacgccccccaccccttccggctcacacccccgcccacactcctgagtggtgcggtgcagcgtcggccgaggcagcagagccgaggagagcaggtccacacctctgcatccctgcaccaggactcaccaagacgcccatcctggaaaaggtcccccgtaagatggcagcaaaaactcccagcagtgaggagtctgggctgcccaaactgcccgtgcccccgctgcagcagaccctggccacgtacctgcagtgcatgcgacacttggtgtctgaggagcagttcaggaagagccaggccattgtgcagcagtttggggcccctggtggcctcggcgagaccctgcagcagaaactcctggagcggcaggagaagacagccaactgggtgtctgagtactggctgaatgacatgtatctcaacaaccgcctggccctgcctgtcaactccagccctgccgtgatctttgctcggcagcacttccctggcaccgatgaccagctgaggtttgcagccagcctcatctctggtgtactcagctacaaggccctgctggacagccactccattcccactgactgtgccaaaggccagctgtcagggcagcccctttgcatgaagcaatactatgggctcttctcctcctaccggctccccggccatacccaggacacgctggtggctcagaacagcagcatcatgccggagcctgagcacgtcatcgtagcctgctgcaatcagttctttgtcttggatgttgtcattaatttccgccgtctcagtgagggggatctgttcactcagttgagaaagatagtcaaaatggcttccaacgaggacgagcgtttgcctccaattggcctgctgacgtctgacgggaggagcgagtgggccgaggccaggacggtcctcgtgaaagactccaccaaccgggactcgctggacatgattgagcgctgcatctgccttgtatgcctggacgcgccaggaggcgtggagctcagcgacacccacagggcactccagctccttcacggcggaggctacagcaagaacggggccaatcgctggtacgacaagtccctgcagtttgtggtgggccgagacggcacctgcggtgtggtgtgcgaacactccccattcgatggcatcgtcctggtgcagtgcactgagcatctgctcaagcacgtgacgcagagcagcaggaagctgatccgagcagactccgtcagcgagctccccgccccccggaggctgcggtggaaatgctccccggaaattcaaggccacttagcctcctcggcagaaaaacttcaacgaatagtaaagaaccttgacttcattgtctataagtttgacaactatgggaaaacattcattaagaagcagaaatgcagccctgatgccttcatccaggtggccctccagctggccttctacaggctccatcgaagactggtgcccacctacgagagcgcgtccatccgccgattccaggagggacgcgtggacaacatcagatcggccactccagaggcactggcttttgtgagagccgtgactgaccacaaggctgctgtgccagcttctgagaagcttctgctcctgaaggatgccatccgtgcccagactgcatacacagtcatggccataacagggatggccattgacaaccacctgctggcactgcgggagctggcccgggccatgtgcaaggagctgcccgagatgttcatggatgaaacctacctgatgagcaaccggtttgtcctctccactagccaggtgcccacaaccacggagatgttctgctgctatggtcctgtggtcccaaatgggtatggtgcctgctacaacccccagccagagaccatccttttctgcatctctagctttcacagctgcaaagagacttcttctagcaagtttgcaaaagctgtggaagaaagcctcattgacatgagagacctctgcagtctgctgccgcctactgagagcaagccattggcaacaaaggaaaaagccacgaggcccagccagggacaccaaccttgactcctgccactaggtttcacctcccaaacccagcctctagaacagccagaccctgcag

SEQ ID NO: 30 - DNA, 1199 bp, Homo sapiens
gcgcggaggccaggagctgagcggcggcggctgccggacgatgggagcgtgagcaggacggtgataacctctccccgatcgggttgcgagggcgccgggcagaggccaggacgcgagccgccagcggcgggacccatcgacgacttcccggggcgacaggagcagccccgagagccagggcgagcgcccgttccaggtggccggaccgcccgccgcgtccgcgccgcgctccctgcaggcaacgggagacgcccccgcgcagcgcgagcgcctcagcgcggccgctcgctctccccatcgagggacaaacttttcccaaacccgatccgagcccttggaccaaactcgcctgcgccgagagccgtccgcgtagagcgctccgtctccggcgagatgtccgagcgcaaagaaggcagaggcaaagggaagggcaagaagaaggagcgaggctccggcaagaagccggagtccgcggcgggcagccagagcccagccttgcctccccgattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagagtctcccattagaatatcagtatccacagaaggagcaaatacttcttcatctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacagtacgtccactccctttctgtctctgcctgaataggagcatgctcagttggtgctgctttcttgttgctgcatctcccctcagattccacctagagctagatgtgtcttaccaga

SEQ ID NO: 31 - DNA, 52 bp, Artificial Sequence
  Description of Artificial Sequence Synthetic oligonucleotide
agcaggatagctcatatttttagaacmattcctcaccaaatggaataattcc

SEQ ID NO: 32 - DNA, 52 bp, Artificial Sequence
  Description of Artificial Sequence Synthetic oligonucleotide
tttctaaactcaattcaatagcacttwgttctactgttaattgaaaaactct

SEQ ID NO: 33 - DNA, 51 bp, Artificial Sequence
  Description of Artificial Sequence Synthetic oligonucleotide
gagcagagacttcctcagacccaaccstctccaggattcagcagcagcacc

SEQ ID NO: 34 - protein, 475 aa, Homo sapiens
MVFLSGNASDSSNCTQPPAPVNISKAILLGVILGGLILFGVLGNILVILSVACHRHLHSVTHYYIVNLAVADLLLTSTVLPFSAIFEVLGYWAFGRVFCNIWAAVDVLCCTASIMGLCIISIDRYIGVSYPLRYPTIVTQRRGLMALLCVWALSLVISIGPLFGWRQPAPEDETICQINEEPGYVLFSALGSFYLPLAIILVMYCRVYVVAKRESRGLKSGLKTDKSDSEQVTLRIHRKNAPAGGSGMASAKTKTHFSVRLLKFSREKKAAKTLGIVVGCFVLCWLPFFLVMPIGSFFPDFKPSETVFKIVFWLGYLNSCINPIIYPCSSQEFKKAFQNVLRIQCLCRKQSSKHALGYTLHPPSQAVEGQHKDMVRIPVGSRETFYRISKTDGVCEWKFFSSMPRGSARITVSKDQSSCTTARTKSRSVTRLECSGMILAHCNLRLPGSRDSPASASQAAGTTGMCHQADATRPS

SEQ ID NO: 35 - DNA, 2543 bp, Homo sapiens
cggacccgaggagcaggaagcggcggctccttcggccacccaggcagcagccacagcggggagtgcgcggcgcggggacaggaagagaggggcaatggctgccgaccccaccgagctgcggctgggcagcctccccgtcttcacccgcgacgacttcgagggcgactggcgcctagtggccagcggcggcttcagccaggtgttccaggcgcggcacaggcgctggcggacggagtacgccatcaagtgcgccccctgccttccacccgacgccgccagctctgatgtgaattacctcattgaagaagctgccaaaatgaagaagatcaagtttcagcacatcgtgtctatctacggggtgtgcaagcagcccctgggtattgtgatggagtttatggccaacggctccctggagaaggtgctgtccacccacagcctctgctggaagctcaggttccgcatcatccatgagaccagcttggccatgaacttcctgcacagcattaagccgcctctgctccacctggacctcaagccgggcaacatactcctggacagcaacatgcatgtcaaaatttcagacttcggcctgtccaagtggatggaacagtccacccggatgcagtacatcgagaggtcggctctgcggggcatgctcagctacatcccccctgagatgttcctggagagtaacaaggccccaggacctaaatatgatgtgtacagctttgcaattgtcatctgggagctactcactcagaagaaaccatactcagggttcaacatgatgatgattattatccgagtggcggcaggcatgcggccctccctacagcctgtctctgaccaatggccaagcgaggcccagcagatggtggacctgatgaaacgctgctgggaccaggaccccaagaagaggccatgctttctagacattaccatcgagacagacatactgctgtcactgctgcagagtcgtgtggcagtcccagagagcaaggccctggccaggaaggtgtcctgcaagctgtcgctgcgccagcccggggaggttaatgaggacatcagccaggaactgatggacagtgactcaggaaactacctgaagcgggcccttcagctctccgaccgtaagaatttggtcccgagagatgaggaactgtgtatctatgagaacaaggtcacccccctccacttcctggtggcccagggcagtgtggagcaggtgaggttgctgctggcccacgaggtagacgtggactgccagacggcctctggatacacgcccctcctgatcgccgcccaggaccagcaacccgacctctgtgccctgcttttggcacatggtgctgatgccaaccgagtggatgaggatggctgggccccactgcactttgcagcccagaatggggatgacggcactgcgcgcctgctcctggaccacggggcctgtgtggatgcccaggaacgtgaagggtggacccctcttcacctggctgcacagaataactttgagaatgtggcacggcttctggtctcccgtcaggctgaccccaacctgcatgaggctgagggcaagacccccctccatgtggccgcctactttggccatgttagcctggtcaagctgctgaccagccagggggctgagttggatgctcagcagagaaacctgagaacaccactgcacctggcagtagagcggggcaaagtgagggccatccaacacctgctgaagagtggagcggtccctgatgcccttgaccagagcggctacggcccactgcacactgcagctgccaggggcaaatacctgatctgcaagatgctgctcaggtacggagccagccttgagctgcccacccaccagggctggacacccctgcatctagcagcctacaagggccacctggagatcatccatctgctggcagagagccacgcaaacatgggtgctcttggagctgtgaactggactcccctgcacctagctgcacgccacggggaggaggcggtggtgtcagcactgctgcagtgtggggctgaccccaatgctgcagagcagtcaggctggacacccctccacctggcggtccagaggagcaccttcctgagtgtcatcaacctcctagaacatcacgcaaatgtccacgcccgcaacaaggtgggctggacacccgcccacctggccgccctcaagggcaacacagccatcctcaaagtgctggtcgaggcaggcgcccagctggacgtccaggatggagtgagctgcacacccctgcaactggccctccgcagccgaaagcagggcatcatgtccttcctagagggcaaggagccgtcagtggccactctgggtggttctaagccaggagccgagatggaaatttagacaacttggccagccgtggtggctcacgtctgtaatcccagcactttgggaggctgaggcaggcagatcacctgatatcaagagtttgaggccagcctggccaacatggcaaaaccctgtctctgctaaaaatacaaaatttagctgggta

SEQ ID NO: 36 - DNA, 25 bp, Artificial Sequence
  Description of Artificial Sequence Synthetic oligonucleotide
tgctgatcgcgctcatctgcactca

SEQ ID NO: 37 - DNA, 25 bp, Artificial Sequence
  Description of Artificial Sequence Synthetic oligonucleotide
ctgcagcgtctccgaggcctgactg

SEQ ID NO: 38 - DNA, 20 bp, Artificial Sequence
  Description of Artificial Sequence Synthetic oligonucleotide
tttcagaggaggccaagaga

SEQ ID NO: 39 - DNA, 20 bp, Artificial Sequence
  Description of Artificial Sequence Synthetic oligonucleotide
ttcaggaggtctggggtaga

SEQ ID NO: 40 - DNA, 20 bp, Artificial Sequence
  Description of Artificial Sequence Synthetic oligonucleotide
ggcgttgccgctctgaatgc

SEQ ID NO: 41 - DNA, 23 bp, Artificial Sequence
  Description of Artificial Sequence Synthetic oligonucleotide
gagggactgagctggacaaccac

SEQ ID NO: 42 - DNA, 20 bp, Artificial Sequence
  Description of Artificial Sequence Synthetic oligonucleotide
gtgagaaagccccaggttac

SEQ ID NO: 43 - DNA, 21 bp, Artificial Sequence
  Description of Artificial Sequence Synthetic oligonucleotide
ccaggctggtctggacttctg

SEQ ID NO: 44 - DNA, 26 bp, Artificial Sequence
  Description of Artificial Sequence Synthetic oligonucleotide
aaggcatcagttttcaatagcttttt

SEQ ID NO: 45 - DNA, 27 bp, Artificial Sequence
  Description of Artificial Sequence Synthetic oligonucleotide
taagtagaaatgggaactctccatctc

SEQ ID NO: 46 - DNA, 17 bp, Artificial Sequence
  Description of Artificial Sequence Synthetic oligonucleotide
tttattttgccaaatat

SEQ ID NO: 47 - DNA, 22 bp, Artificial Sequence
  Description of Artificial Sequence Synthetic oligonucleotide
tctttattttaccaaatatcat

SEQ ID NO: 48 - DNA, 30 bp, Artificial Sequence
  Description of Artificial Sequence Synthetic oligonucleotide
acgttggatggaaagtaagcaggatagctc

SEQ ID NO: 49 - DNA, 30 bp, Artificial Sequence
  Description of Artificial Sequence Synthetic oligonucleotide
acgttggatgtcctcactcccacttttcac

SEQ ID NO: 50 - DNA, 30 bp, Artificial Sequence
  Description of Artificial Sequence Synthetic oligonucleotide
acgttggatgcggaatctgattttctaaac

SEQ ID NO: 51 - DNA, 30 bp, Artificial Sequence
  Description of Artificial Sequence Synthetic oligonucleotide
acgttggatgctcagcctaacgtatctcac

SEQ ID NO: 52 - DNA, 1193 bp, Homo sapiens
taaagaaaacggatacattcgaaagcagctatgaaacatgcactaaggtctaatagggaagctggaaaagcagcactcaagtaatttcaccttagaggcaaaaatgggtgatttctttctgttcatttcatagtttctgagtcctgagaaaggcaaagtttgctttgcttgggtatgtctgctgtcagtaaatggctgcaggagccgaagtggtaaactcctcggtctccagaaatcagaagaaaattttaggaagccccttggcatcacgcacctccctctgggctatggcatctctgagtcagctgagtagccacctgaactacacctgtggggcagagaactccacaggtgccagccaggcccgcccacatgcctactatgccctctcctactgcgcgctcatcctggccatcgtcttcggcaatggcctggtgtgcatggctgtgctgaaggagcgggccctgcagactaccaccaactacttagtagtgagcctggctgtggcagacttgctggtggccaccttggtgatgccctgggtggtatacctggaggtgacaggtggagtctggaatttcagccgcatttgctgtgatgtttttgtcaccctggatgtcatgatgtgtacagccagcatccttaatctctgtgccatcagcatagacaggtacactgcagtggtcatgcccgttcactaccagcatggcacgggacagagctcctgtcggcgcgtggccctcatgatcacggccgtctgggtactggcctttgctgtgtcctgccctcttctgtttggctttaataccacaggggaccccactgtctgctccatctccctgagtcccaccatagcgcccaagctcagcttagaagttcgaaaactcagcaatggcagattatcgacatctttgaagctggggcccctgcaacctcggggagtgccacttcgggagaagaaggcaacccaaatggtggccattgtgcttggggccttcattgtctgctggctgcccttcttcttgacccatgttctcaatacccactgccagacatgccacgtgtccccagagctttacagtgccacgacatggctgggctacgtgaatagcgccctcaaccctgtgatctataccaccttcaatatcgagttccggaaagccttcctcaagatcctgtcttgctgagggagc

SEQ ID NO: 53 - DNA, 1984 bp, Homo sapiens
cccgagagcgcccattcacccccctcacccacctccccgcgttcccacttccccgcactctgacccggccggacgcccctcccctatcttgccgcccgccccctccagggggctctgctcccaccccagggagcccatccgacctctgcttgacttcccgccgcttccttcaggggcctcggctcatcgggtgcccctccccaaacttccaacccgtttgctccaggagttcctgccccatccccgagggcgcccaaatagccacactgtgtcctcctgtagtcgccgccccctgacctagcgcgacccagcgcccccgcccatgtccccccactcacctcccccggggggcgtggtgagtcgcggtctgttctcacggacggtccccgtccagcctgcgcttcgccggggccctcatctgctttcccgccaccctatcactcccttgccgtccaccctcggtcctcatggtcccagagccgggcccaaccgccaatagcaccccggcctggggggcagggccgccgtcggccccggggggcagcggctgggtggcggccgcgctgtgcgtggtcatcgcgctgacggcggcggccaactcgctgctgatcgcgctcatctgcactcagcccgcgctgcgcaacacgtccaacttcttcctggtgtcgctcttcacgtctgacctgatggtggggctggtggtgatgccgccggccatgctgaacgcgctgtacgggcgctgggtgctggcgcgcggcctctgcctgctctggaccgccttcgacgtgatgtgctgcagcgcctccatcctcaacctctgcctcatcagcctggaccgctacctgctcatcctctcgccgctgcgctacaagctgcgcatgacgcccctgcgtgccctggccctagtcctgggcgcctggagcctcgccgctctcgcctccttcctgcccctgctgctgggctggcacgagctgggccacgcacggccacccgtccctggccagtgccgcctgctggccagcctgccttttgtccttgtggcgtcgggcctcaccttcttcctgccctcgggtgccatatgcttcacctactgcaggatcctgctagctgcccgcaagcaggccgtgcaggtggcctccctcaccaccggcatggccagtcaggcctcggagacgctgcaggtgcccaggaccccacgcccaggggtggagtctgctgacagcaggcgtctagccacgaagcacagcaggaaggccctgaaggccagcctgacgctgggcatcctgctgggcatgttctttgtgacctggttgcccttctttgtggccaacatagtccaggccgtgtgcgactgcatctccccaggcctcttcgatgtcctcacatggctgggttactgtaacagcaccatgaaccccatcatctacccactcttcatgcgggacttcaagcgggcgctgggcaggttcctgccatgtccacgctgtccccgggagcgccaggccagcctggcctcgccatcactgcgcacctctcacagcggcccccggcccggccttagcctacagcaggtgctgccgctgcccctgccgccggactcagattcggactcagacgcaggctcaggcggctcctcgggcctgcggctcacggcccagctgctgcttcctggcgaggccacccaggaccccccgctgcccaccagggccgctgccgccgtcaatttcttcaacatcgaccccgcggagcccgagctgcggccgcatccacttggcatccccacgaactgacccgggcttggggctggccaatggggagctggattgagcagaacccagaccctgagtccttgggccagctcttggctaagaccaggaggctgcaagtctcctagaagccctctgagctccagaggggtgcgcagagctgaccccctgctgccatctccaggccccttacctgcagggatcatagctgactcaga

SEQ ID NO: 54 - DNA, 1854 bp, Homo sapiens
ttggggagttatgcgccagtgccccagtgaccgcgggacacggagaggggaagtctgcgttgtacataaggacctagggactccgagcttggcctgagaacccttggacgccgagtgcttgccttacgggctgcactcctcaactctgctccaaagcagccgctgagctcaactcctgcgtccagggcgttcgctgcgcgccaggacgcgcttagtacccagttcctgggctctctcttcagtagctgctttgaaagctcccacgcacgtcccgcaggctagcctggcaacaaaactggggtaaaccgtgttatcttaggtcttgtcccccagaacatgacctagaggtacctgcgcatgcagatggccgatgcagccacgatagccaccatgaataaggcagcaggcggggacaagctagcagaactcttcagtctggtcccggaccttctggaggcggccaacacgagtggtaacgcgtcgctgcagcttccggacttgtggtgggagctggggctggagttgccggacggcgcgccgccaggacatcccccgggcagcggcggggcagagagcgcggacacagaggcccgggtgcggattctcatcagcgtggtgtactgggtggtgtgcgccctggggttggcgggcaacctgctggttctctacctgatgaagagcatgcagggctggcgcaagtcctctatcaacctcttcgtcaccaacctggcgctgacggactttcagtttgtgctcaccctgcccttctgggcggtggagaacgctcttgacttcaaatggcccttcggcaaggccatgtgtaagatcgtgtccatggtgacgtccatgaacatgtacgccagcgtgttcttcctcactgccatgagtgtgacgcgctaccattcggtggcctcggctctgaagagccaccggacccgaggacacggccggggcgactgctgcggccggagcctgggggacagctgctgcttctcggccaaggcgctgtgtgtgtggatctgggctttggccgcgctggcctcgctgcccagtgccattttctccaccacggtcaaggtgatgggcgaggagctgtgcctggtgcgtttcccggacaagttgctgggccgcgacaggcagttctggctgggcctctaccactcgcagaaggtgctgttgggcttcgtgctgccgctgggcatcattatcttgtgctacctgctgctggtgcgcttcatcgccgaccgccgcgcggcggggaccaaaggaggggccgcggtagccggaggacgcccgaccggagccagcgcccggagactgtcgaaggtcaccaaatcagtgaccatcgttgtcctgtccttcttcctgtgttggctgcccaaccaggcgctcaccacctggagcatcctcatcaagttcaacgcggtgcccttcagccaggagtatttcctgtgccaggtatacgcgttccctgtgagcgtgtgcctagcgcactccaacagctgcctcaaccccgtcctctactgcctcgtgcgccgcgagttccgcaaggcgctcaagagcctgctgtggcgcatcgcgtctccttcgatcaccagcatgcgccccttcaccgccactaccaagccggagcacgaggatcaggggctgcaggccccggcgccgccccacgcggccgcggagccggacctgctctactacccacctggcgtcgtggtctacagcggggggcgctacgacctgctgcccagcagctctgcctactgacgcaggcctcaggcccagggcgcgccgtcggggcaaggtggccttccccgggcggtaaagaggtgaaaggatgaaggagggctgggg

SEQ ID NO: 55 - DNA, 1146 bp, Homo sapiens
accaatctctgatgccctgcgatgcccacactcaatacttctgcctctccacccacattcttctgggccaatgcctccggaggcagtgtgctgagtgctgatgatgctccgatgcctgtcaaattcctagccctgaggctcatggttgccctggcctatgggcttgtgggggccattggcttgctgggaaatttggcggtgctgtgggtactgagtaactgtgcccggagagcccctggcccaccttcagacaccttcgtcttcaacctggctctggcggacctgggactggcactcactctccccttttgggcagccgagtcggcactggactttcactggcccttcggaggtgccctctgcaagatggttctgacggccactgtcctcaacgtctatgccagcatcttcctcatcacagcgctgagcgttgctcgctactgggtggtggccatggctgcggggccaggcacccacctctcactcttctgggcccgaatagccaccctggcagtgtgggcggcggctgccctggtgacggtgcccacagctgtcttcggggtggagggtgaggtgtgtggtgtgcgcctttgcctgctgcgtttccccagcaggtactggctgggggcctaccagctgcagagggtggtgctggctttcatggtgcccttgggcgtcatcaccaccagctacctgctgctgctggccttcctgcagcggcggcaacggcggcggcaggacagcagggtcgtggcccgctctgtccgcatcctggtggcttccttcttcctctgctggtttcccaaccatgtggtcactctctggggtgtcctggtgaagtttgacctggtgccctggaacagtactttctatactatccagacgtatgtcttccctgtcactacttgcttggcacacagcaatagctgcctcaaccctgtgctgtactgtctcctgaggcgggagccccggcaggctctggcaggcaccttcagggatctgcggttgaggctgtggccccagggcggaggctgggtgcaacaggtggccctaaagcaggtaggcaggcggtgggtcgcaagcaacccccgggagagccgcccttctaccctgctcaccaacctggacagagggacacccgggtga